SEQUENCE LISTING (1 sequence)

SEQ ID NO: 1 - protein, 9 aa, synthetic construct
RPRPDDLEI

(19) United States
(12) Patent Application Publication
(10) Pub. No.: US 2026/0014085 A1
(43) Pub. Date: Jan. 15, 2026

(54) MILK DERIVED EXOSOMES AND USES THEREOF

(71) Applicant: VIRGINIA TECH INTELLECTUAL PROPERTIES, INC., BLACKSBURG, VA (US)

(72) Inventors: Robert G. GOURDIE, Blacksburg, VA (US); Linda Jane JOURDAN, Blacksburg, VA (US); Spencer MARSH, Blacksburg, VA (US)

(73) Assignee: VIRGINIA TECH INTELLECTUAL PROPERTIES, INC., BLACKSBURG, VA (US)

(21) Appl. No.: 18/995,062

(22) PCT Filed: Jul. 14, 2023
(86) PCT No.: PCT/US2023/070282

§ 371 Date: Jan. 15, 2025

Related U.S. Application Data
(60) Provisional application No. 63/389,744, filed on Jul. 15, 2022.

(51) Int. Cl. A61K 9/50; A61K 35/20; A61K 38/08; A61K 39/00; A61K 39/395; A61P 17/02

(52) U.S. Cl. CPC A61K 9/5063; A61K 35/20; A61K 38/08; A61K 39/395; A61P 17/02; A61K 2039/505

(57) ABSTRACT

Described herein are milk exosomes and uses thereof. In some embodiments, the milk exosomes are capable of targeting an injury site or a cancer cell or population thereof. The milk exosomes can contain an exogenous cargo. Described herein are formulations containing the milk exosomes, and optionally, a targeting agent, such as IgG or stimulation of ATP concentration, and/or ADP. Also described herein are methods of delivering a cargo to a target.

### CROSS-REFERENCE TO RELATED APPLICATIONS

This application claims the benefit of and priority to U.S. Provisional Patent Application No. 63/389,744, filed on Jul. 15, 2022, entitled MILK DERIVED EXOSMES AND USES THEREOF,” the contents of which is incorporated by reference herein in its entirety.

### STATEMENT REGARDING FEDERALLY SPONSORED RESEARCH

This invention was made with government support under Grant No. HL161237, HL141855, and HL056728 awarded by the National Institutes of Health. The government has certain rights in the invention.

### SEQUENCE LISTING

This application contains a sequence listing filed in electronic form as an xml file entitled “VTIP-0390WP_ST26.xml”, created on Jul. 14, 2023, and having a size of 1,753 bytes. The content of the sequence listing is incorporated herein in its entirety.

### TECHNICAL FIELD

The subject matter disclosed herein is generally directed to milk derived exosomes and uses thereof.

### BACKGROUND

Small extracellular vesicles (sEVs), such as exosomes, are produced from the phospholipid bilayer of almost every cell in the body and are present in high quantities in milk. Milk exosomes, such as bovine milk exosomes, can be loaded with cargos, such as therapeutic cargos. Milk exosomes present a vehicle for oral drug delivery. sEVs, such as exosomes, are absorbed systemically and can cross barriers intact. See e.g., Betker, Jamie L., et al. “The Potential of Exosomes From Cow Milk for Oral Delivery.” Journal of Pharmaceutical Sciences, vol. 108, no. 4, 2019, pp. 1496-1505; Manca, Sonia et al. “Milk exosomes are bioavailable and distinct microRNA cargos have unique tissue distribution patterns.” Scientific reports vol. 8,1 11321. 27 Jul. 2018; and Munagala, Radha et al. “Bovine milk-derived exosomes for drug delivery.” Cancer letters vol. 371,1 (2016): 48-61. doi:10.1016/j.canlet.2015.10.020. However, it is still unknown how milk exosomes are capable of doing this and further how such mechanisms may be employed or modified for development of clinically relevant delivery vehicles.

Citation or identification of any document in this application is not an admission that such a document is available as prior art to the present invention.

### SUMMARY

Described in certain example embodiments herein are milk exosomes comprising an exogenous cargo, wherein the milk exosome is capable of targeting an injury or site thereof or a cancer cell or cancer cell population by targeting or stimulating ATP secretion and/or concentration and/or neonatal Fc receptor (FcRN).

In certain example embodiments, the injury is a mechanical injury or a non-mechanical injury. In certain example embodiments, the non-mechanical injury is a chemical injury, electrical injury, radiation injury, an inflammatory injury, or an ischemic injury. In certain example embodiments, the mechanical injury is a wound or burn.

In certain example embodiments, the injury or site thereof has a greater concentration of ATP than a non-injury or site thereof.

In certain example embodiments, the cancer cell or cancer cell population has or has a microenvironment that has a greater concentration of ATP than a microenvironment of a non-cancer cell or non-cancer cell population.

In certain example embodiments, the cancer cell or cancer cell population secretes ATP.

In certain example embodiments, the milk exosome does not contain an exogenous targeting moiety.

In certain example embodiments, the milk exosome comprises connexin 43. In certain example embodiments, the connexin 43 is a native connexin 43 or wherein the connexin 43 is an engineered connexin 43.

In certain example embodiments, the exogenous cargo is a biologic molecule. In certain example embodiments, the biologic molecule is a polypeptide, peptide, or a nucleic acid. In certain example embodiments, the exogenous cargo is an ACT1 peptide, ACT11 peptide, an ACT minus peptide, a selectide, or any combination thereof. In certain example embodiments, the milk exosome is a bovine milk exosome.

In certain example embodiments, the milk exosome, prior to containing the exogenous cargo, is isolated according to a method comprising (a) centrifuging a mammalian milk under conditions suitable to separate fats from one or more other components of the mammalian milk; (b) removing the separated fats from the mammalian milk; (c) after step (b) centrifuging the remaining mammalian milk one or more times and skimming any noticeable separated fats after each centrifuging in step (c); (d) filtering the remaining biological fluid after step (c); (e) optionally performing one or more ultracentrifugation steps after (d); (f) chelating divalent cations with about 10 mM to about 100 mM EDTA at about 30-42 degrees Celsius after (d) or optionally (e) and optionally for about 15-120 minutes; and (g) after (f), optionally performing tangential flow filtration to obtain a retentate, wherein the retentate is optionally ultracentrifuged via one or more ultracentrifugation steps or stored at −80 degrees C., and separating out fractions of the retentate, optionally via column separation, after the retentate is optionally ultracentrifuged or stored at −80 degrees C., wherein the method comprises step (e) or step (g) but not both.

Described in certain example embodiments herein are populations of milk exosomes comprising one or more milk exosomes of the present disclosure described in greater detail elsewhere herein. In certain example embodiments, the population is enriched for connexin 43 positive milk exosomes. In certain example embodiments, the milk exosome, prior to containing the exogenous cargo, is isolated according to a method comprising (a) centrifuging a mammalian milk under conditions suitable to separate fats from one or more other components of the mammalian milk; (b) removing the separated fats from the mammalian milk; (c) after step (b) centrifuging the remaining mammalian milk one or more times and skimming any noticeable separated fats after each centrifuging in step (c); (d) filtering the remaining biological fluid after step (c); (e) optionally performing one or more ultracentrifugation steps after (d); (f) chelating divalent cations with about 10 mM to about 100 mM EDTA at about 30-42 degrees Celsius after (d) or optionally (e) and optionally for about 15-120 minutes; and (g) after (f), optionally performing tangential flow filtration to obtain a retentate, wherein the retentate is optionally ultracentrifuged via one or more ultracentrifugation steps or stored at −80 degrees C., and separating out fractions of the retentate, optionally via column separation, after the retentate is optionally ultracentrifuged or stored at −80 degrees C., wherein the method comprises step (e) or step (g) but not both. In certain example embodiments, the method further comprises enriching the milk exosomes for connexin 43 expressing milk exosomes. In certain example embodiments, the milk exosomes do not contain an exogenous targeting moiety. In certain example embodiments, the exogenous cargo is a biologic molecule. In certain example embodiments, the biologic molecule is a polypeptide, peptide, or a nucleic acid. In certain example embodiments, the exogenous cargo is an ACT1 peptide, ACT11 peptide, an ACT minus peptide, a selectide, or any combination thereof.

Described in certain example embodiments herein are pharmaceutical formulations comprising a milk exosome of the present disclosure described in greater detail elsewhere herein or a population thereof and a pharmaceutically acceptable carrier. In certain example embodiments, the pharmaceutical formulation further comprises an agent capable of stimulating ATP release, secretion, and/or production, ATP, ADP, or any combination thereof. In certain example embodiments, the pharmaceutical formulation is in a dosage form that releases the agent capable of stimulating ATP release, secretion, and/or production, ATP, ADP, or any combination thereof prior to the milk exosome or population thereof thereby delivering the agent capable of stimulating ATP release, secretion, and/or production, ATP, ADP, or any combination thereof prior to the milk exosome.

In certain example embodiments, the pharmaceutical formulation further comprises one or more immunoglobulins. In certain example embodiments, the one or more immunoglobulins are selected from IgG, IgM, IgA, IgD, and/or IgE. In certain example embodiments, the pharmaceutical formulation is in a dosage form that releases the immunoglobulin prior to the milk exosome or population thereof thereby delivering the immunoglobulin prior to the milk exosome.

Described in certain example embodiments herein are methods of treating an injury at an injury site or treating a cancer in a subject in need thereof comprising: administering to the subject in need thereof, a milk exosome of the present disclosure as described in greater detail elsewhere herein or a population thereof or a pharmaceutical formulation comprising the milk exosome of the present disclosure described in greater detail elsewhere herein or the population thereof. In certain example embodiments, the milk exosome is a bovine milk exosome. In certain example embodiments, the milk exosome does not contain an exogenous targeting moiety. In certain example embodiments, the milk exosome comprises connexin 43. In certain example embodiments, the connexin 43 is a native connexin 43 or wherein the connexin 43 is an engineered connexin 43. In certain example embodiments, the exogenous cargo is a biologic molecule. In certain example embodiments, the biologic molecule is a polypeptide, peptide, or a nucleic acid. In certain example embodiments, the exogenous cargo is an ACT1 peptide, ACT11 peptide, an ACT minus peptide, a selectide, or any combination thereof.

In certain example embodiments, the method further comprises administering an amount of one or more immunoglobulins to the subject in need thereof. In certain example embodiments, administering the amount of one or more immunoglobulins to subject in need thereof occurs prior to administering the amount of a milk exosome of the present disclosure as described in greater detail elsewhere herein or a population thereof or a pharmaceutical formulation comprising the milk exosome of the present disclosure as described in greater detail elsewhere herein or the population thereof. In certain example embodiments, the one or more immunoglobulins are selected from are selected from IgG, IgM, IgA, IgD, and/or IgE. In certain example embodiments, the one or more immunoglobulins consists of or comprises IgG. In certain example embodiments, the one or more immunoglobulins increase uptake of the milk exosome or population thereof in a cell. In certain example embodiments, the cell is a polarized cell. In certain example embodiments, wherein the cell is a gastrointestinal cell, a heart cell, a liver cell, brain cell, nerve cell, reproductive cell, cancer cell, or any combination thereof. In certain example embodiments, the method further comprises administering one or more agents that stimulate ATP release, secretion, and/or production, ATP, ADP, or any combination thereof to the subject in need thereof. In certain example embodiments, administering the one or more agents that stimulate ATP release, secretion, and/or production, ATP, ADP, or any combination thereof to subject in need thereof occurs prior to administering the amount of a milk exosome of the present disclosure as described in greater detail elsewhere herein or a population thereof or a pharmaceutical formulation comprising the milk exosome of the present disclosure as described in greater detail elsewhere herein or the population thereof or a pharmaceutical formulation comprising the milk exosome of the present disclosure as described in greater detail elsewhere herein. In certain example embodiments, wherein the one or more agents that stimulate ATP release, secretion, and/or production, ATP, ADP, or any combination thereof increases the cellular uptake of the milk exosome or population thereof in a cell. In certain example embodiments, the cell is a polarized cell. In certain example embodiments, the cell is a gastrointestinal cell, heart cell, liver cell, brain cell, nerve cell, reproductive cell, cancer cell, or any combination thereof. In certain example embodiments, the injury is a mechanical injury site or a non-mechanical injury. In certain example embodiments, the non-mechanical injury is a chemical injury, electrical injury, radiation injury, or an ischemic injury. In certain example embodiments, the mechanical injury is a wound or burn.

In certain example embodiments, the milk exosome or population thereof and/or pharmaceutical formulation are irradiated, sterilized, or both.

Described in certain example embodiments herein are methods of treating a wound or cancer in a subject comprising administering a milk exosome and an agent capable of stimulating ATP release, secretion, and/or production to the subject in need thereof. In certain example embodiments, the milk exosome is a milk exosome of the present disclosure as described in greater detail elsewhere herein.

In certain example embodiments, the milk exosome or population thereof and/or pharmaceutical formulation thereof of the present disclosure as described in greater detail elsewhere herein are irradiated, sterilized, or both.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1Q—Milk-Derived Extracellular Vesicles target surgically-damaged dermal tissue (FIGS. 1A-1E and 1P) and ischemic cardiac tissue (1F-1P and 1Q) in vivo in an LAD-induced cardiac ischemia model. Tissues are stained for actin expression with FITC-Phalloidin (Green, as represented in greyscale) and nuclear expression with Hoechst stain (Blue, as represented in greyscale); mEV's are tagged with Cell-Tracker Deep Red (Yellow, as represented in greyscale). (FIG. 1A-1D) Surgical Skin from cardiac ischemia model—skin at surgical site was removed during organ explant, stained and imaged—FIG. 1A represents wide angle of skin specimen, with FIG. 1B at 20× further zoom, FIG. 1C at high magnification 63× enhanced zoom, while FIG. 1D is a duplication of FIG. 1C with no Actin signal, showing high levels of mEV's present at the wound site. (FIG. 1E-1H) Remote Skin from cardiac ischemia model—skin on opposite side of the body from surgical site was removed during organ explant, stained and imaged—FIG. 1E represents a wide angle of skin specimen, with FIG. 1F at 20× further zoom, FIG. 1G at high magnification 63× enhanced zoom, while FIG. 1H is a duplication of FIG. 1G with no actin signal, showing a complete lack of mEVs present at remote skin site. (FIG. 1P) Quantification of mEV expression in skin samples—Data normalized via particle counts of mEV's divided by total nuclei per image for final data units of mEV's/Cell for each image—Control indicates mouse with no surgery, Sham surgical is a surgical skin site with no ischemia induced, Sham remote is a remote skin site with no ischemia induced, LAD surgical is a surgical skin site with ischemia induced, LAD remote is a remote skin site with ischemia induced, while (−) CTL LAD remote and surgical are specimens with no mEV's added during ischemia surgery. High levels of significance indicates mEV's are capable of targeting injured sites in dermal wound healing. (FIG. 1I-1L) Low magnification overview of ischemic heart specimen—boxes indicate ischemic zone (E, upper left box in FIG. 1I) and Remote zone (F, lower right box in FIG. 1I). FIG. 1J represents 40× image of ischemic cardiac tissue, FIG. 1K is a high magnification 63× zoom of ischemic cardiac tissue, while FIG. 1L is a duplication of FIG. 1K with no actin signal, showing high levels of mEV's present in ischemic cardiac tissue after LAD-induced cardiac ischemia. FIG. 1M represents 40× image of remote zone cardiac tissue, FIG. 1N is a high magnification 63× zoom of remote zone cardiac tissue, while FIG. 1O is a duplication of FIG. 1N with no actin signal, showing a complete lack of mEV signal in cardiac tissue distant from the ischemic insult. This suggests not only tissue targeting during injury, but an elegant targeting system which can differentiate wounded and unwounded zones within tissues. (FIG. 1Q) Quantification of mEV expression in cardiac samples—Control indicates mouse with no surgery, Sham is a mouse heart which received every surgical procedure except the artery blockage, IR+Exo is ischemic cardiac tissue in an injured model, and IR no exo is a mouse which underwent LAD ischemia without any mEV's added, confirming no background signal is responsible for results. High levels of significance indicates mEV's are capable of targeting injured sites in cardiac wound healing.

FIG. 2A-2E—Milk-Derived Extracellular Vesicles target injured human dermal fibroblasts in vitro in a scratch wound model. Cells are cultured to confluency, then scratched in low serum conditions (1% NCS), rinsed, and supplied fresh medium with 20 μg/mL CTDR-Tagged mEV's. (FIG. 2A) Unwounded huDFs at standard magnification. (FIG. 2B) 63× magnification image of Unwounded huDFs given CTDR tagged mEV's. (FIG. 2C) Scratch-Wounded huDFs at standard magnification. (FIG. 2D) 63× magnification image of Scratch-Wounded huDFs, showing high levels of mEV's taken up into cells. (FIG. 2E) Quantification of mEV expression in huDFs—Data normalized via particle counts of mEV's divided by total nuclei per image for final data units of mEV's/Cell for each image—Data clearly shows statistically significant increase in mEV/Cell expression, with a 20+ Fold increase in wounded situations.

FIG. 3A-3E—Milk-Derived Extracellular Vesicles contain endogenous Connexin-43. (FIG. 3A, left) Western blotting for Cx43 C-tail using Antibody 71-0700 yields positive expression of Cx43 at about 42.0 kDa. Positive marker for HeLa cells shown in middle lane with expression at about 40 kDa. (FIG. 3A, right) Western blotting for Cx43 C-Terminus using Antibody C6219 yields negative expression of Cx43 for mEV's. Molecular Weight ladder shown with bands expressing between 50 and 37 kDa. Densitometry software used to determine molecular weights of unknown bands. (FIG. 3B-3C) Nanogold TEM for Cx43 C-tail using Antibody 71-0700 yields positive expression of Cx43, present in approximately 20% of mEV's in any given field. Illustrates positive expression of Cx43 in mEVs under TEM imaging with uralyness counterstaining. (FIG. 3D) Nanogold TEM for Cx43 C-Terminus using Antibody C6219 yields negative expression for Cx43 in mEV's. (FIG. 3E) negative control, Low signal with background on non-vesicle. The culmination of these images reveals a truncated version of Cx43 present in mEV's, which may have a role in downstream function. The potential truncation may result in free-CT AA sequences.

FIG. 4A-4H—Milk-Derived Extracellular Vesicles target injured MDCK cells in vitro in a scratch wound model and exhibit differential targeting capabilities dependent upon target cell Connexin-43 Expression. Cells are cultured to confluency, then scratched in low serum conditions (1% NCS), rinsed, and supplied fresh medium with 20 μg/mL CTDR-Tagged mEV's. (FIG. 4A) Quantification of mEV expression in MDCKs—Data normalized via particle counts of mEV's divided by total nuclei per image for final data units of mEV's/Cell for each image—Data shows clear statistically significant increase in mEV/cell expression, with a near 40 fold increase for wounded cells with enhanced Cx43 expression, tying the wounded response to cellular Cx43. (FIG. 4B) Unwounded Parent (Low Cx43) MDCKs; (FIG. 4C) Wounded Parent (Low Cx43) MDCKs; (FIG. 4D) Unwounded B5 Clone (High Cx43) MDCKs; (FIG. 4E) Wounded B5 Clone (High Cx43) MDCKs. (FIG. 4F) Quantification of mEV expression in B5 Clone (High Cx43) MDCK's with and without added Gap27, showing a statistical drop in mEV expression when Cx43 activity is blocked with Gap27. (FIG. 4G) Wounded B5 Clone (High Cx43) MDCKs provided mEV's; (FIG. 4H) Wounded B5 Clone (High Cx43) MDCKs provided mEV's and Gap27, a Connexin inhibitor.

FIG. 5A-5C—Milk-Derived Extracellular Vesicle targeting capabilities to high Cx43 expressing B5 Clone MDCKS are completely eliminated via use of exogenous Apyrase. Cells are cultured to confluency, then scratched in low serum conditions (1% NCS), rinsed, and supplied fresh medium with 20 μg/mL CTDR-Tagged mEV's with or without 100 uM Apyrase. (FIG. 5A) Wounded B5 Clone (High Cx43) given CTDR-Tagged mEV's; (FIG. 5B) Wounded B5 Clone (High Cx43) given CTDR tagged mEV's with 100 M Apyrase. (FIG. 5C) Quantification of mEV expression in B5 Clone (High Cx43) MDCKs with and without added apyrase, indicating a significant and complete elimination of mEV expression when ATP secretion by injured cells is quenched.

FIG. 6A-6G—Milk-Derived Extracellular Vesicles contain endogenous bioactive capabilities in a Scratch-Wounded model of wound healing in Human dermal Fibroblasts. Cells are cultured to confluency, then scratched in low serum conditions (1% NCS), rinsed, and supplied fresh medium with 20 μg/mL mEV's, 100 uM CT-Peptide, or 20 uL Vehicle (HEPES buffer). (FIG. 6A) Starting time point post-scratch-vehicle; (FIG. 6B) Final time point post-scratch-vehicle. (FIG. 6C) Starting time point post-scratch-mEV's; (FIG. 6D) Final time point post-scratch-mEV's. (FIG. 6E) Starting time point post-scratch—100 uM CT peptide; (FIG. 6F) Final time point post-scratch—100 uM CT peptide. (FIG. 6G) Quantification of wound closure using ImageJ border zone analysis plugin software. Scratches are scanned and imaged at t=0 and scratch size is quantified, then after 8 hours, cells are stopped, fixed and imaged again, then final scratch size is quantified. Migration index is calculated by subtracting the final area from the starting and dividing by the average of vehicle control samples, giving a value of 1.0 for vehicle control. mEV's contain endogenous bioactivity equal to nearly 40 uM free CT peptide (aCT1), which is in phase III clinical trials for dermal wound closure.

FIG. 7A-7L—Milk-Derived Extracellular Vesicles contain endogenous bioactive capabilities in a Scratch-Wounded model of wound healing in Cx43-variable MDCK cells. Cells are cultured to confluency, then scratched in low serum conditions (1% NCS), rinsed, and supplied fresh medium with 20 μg/mL mEV's or 20 uL Vehicle (HEPES buffer). (FIG. 7A) Quantification of wound closure using ImageJ border zone analysis plugin software. Scratches are scanned and imaged at t=0 and scratch size is quantified, then after 8 hours, cells are stopped, fixed and imaged again, then final scratch size is quantified. Migration index is calculated by subtracting the final area from the starting and dividing by the average of vehicle control samples, giving a value of 1.0 for vehicle control. mEV's contain endogenous bioactivity that is Cx43 Dependent, with over 8× healing potential when cells contain enhanced Cx43 levels. (FIG. 7B) Starting time point post-scratch-representative image; (FIG. 7C) Parent (Low Cx43) MDCK final time point post-scratch-vehicle; (FIG. 7D) B5 Clone (high Cx43) MDCK final time point post-scratch-Vehicle; (FIG. 7E) Parent (low Cx43) MDCK final time point post-scratch-mEV's; (FIG. 7F) B5 Clone (High Cx43) MDCK final time point-post scratch-mEV's. (FIG. 7G) Quantification of wound closure using ImageJ border zone analysis plugin software. Scratches are scanned and imaged at t=0 and scratch size is quantified, then after 8 hours, cells are stopped, fixed and imaged again, then final scratch size is quantified. Migration index is calculated by subtracting the final area from the starting and dividing by the average of vehicle control samples, giving a value of 1.0 for vehicle control. mEV's contain significant endogenous bioactivity that is dependent upon activated functional Cx43 activity, suggesting that over 50% of mEV bioactivity is Connexin-43 dependent. (FIG. 7H) B5 Clone (High Cx43) MDCK starting time point scratch-representative image; (FIG. 7I) Final time point post-scratch-Vehicle; (FIG. 7J) Final time point post-scratch-mEV's; (FIG. 7K) Final time point post-scratch-Gap27; (FIG. 7L) Final time point post-scratch-Gap27+mEV's.

FIG. 8A-8G—Milk-Derived Extracellular Vesicle wound healing capabilities to high Cx43 expressing B5 Clone MDCKS are heavily reduced via use of exogenous Apyrase. Cells are cultured to confluency, then scratched in low serum conditions (1% NCS), rinsed, and supplied fresh medium with 20 μg/mL mEV's with or without 100 uM Apyrase or 20 uL Vehicle control. (FIG. 8A) Starting time point post-scratch-vehicle; (FIG. 8B) B5 Clone (high Cx43) MDCK final time point post-scratch-Vehicle; (FIG. 8C) Starting time point post-scratch-mEV's; (FIG. 8D) B5 Clone (high Cx43) MDCK final time point post-scratch-mEV's; (FIG. 8E) Starting time point post-scratch-mEV's+Apyrase; (FIG. 8F) B5 Clone (high Cx43) MDCK final time point post-scratch-mEV's+Apyrase. (FIG. 8G) Quantification of wound closure using ImageJ border zone analysis plugin software. Scratches are scanned and imaged at t=0 and scratch size is quantified, then after 8 hours, cells are stopped, fixed and imaged again, then final scratch size is quantified. Migration index is calculated by subtracting the final area from the starting and dividing by the average of vehicle control samples, giving a value of 1.0 for vehicle control. Apyrase effectively reduces the ability of mEV's to promote wound closure, suggesting a critical role of ATP secretion in mEV uptake and activity.

FIG. 9A-9B—Loading of mEV's with exogenous peptide RPRPDDLEI (SEQ ID NO: 1) results in combination therapeutic. (FIG. 9A) HPLC readout of loaded mEV's contrasted with free 100 uM RPRPDDLEI (SEQ ID NO: 1). Free peptide shown in black line, loaded mEV's in grey line, and blank mEV's shown with yellow line. HPLC readout indicates 35% loading efficiency. (FIG. 9B) Nanoparticle Tracking Analysis results—mEV's were loaded with FAM-conjugated RPRPDDLEI (SEQ ID NO: 1), then read on NTA for both scatter signal and 488 fluorescent signal. Ratio of fluorescently-tagged mEV's to total scatter mEV's yields ˜34% efficiency in loading mEVs with exogenous peptide.

FIG. 10A-10F—RPRPDDLEI-loaded mEV's (SEQ ID NO: 1) exhibit greater wound healing bioactivity than that of blank mEV's, confirming loading of mEVs with highly bioactive peptide RPRPDDLEI (SEQ ID NO: 1). (FIG. 10A) Quantification of wound closure using ImageJ border zone analysis plugin software. Scratches are scanned and imaged at t=0 and scratch size is quantified, then after 8 hours, cells are stopped, fixed and imaged again, then final scratch size is quantified. Migration index is calculated by subtracting the final area from the starting and dividing by the average of vehicle control samples, giving a value of 1.0 for vehicle control. (FIG. 10B) representative image of huDF initial time point post-scratch; (FIG. 10C) huDF final time point post scratch-Vehicle; (FIG. 10D) huDF final time point post-scratch-mEV's; (FIG. 10E) huDF Final time point post-scratch-RPRPDDLEI (SEQ ID NO: 1); (FIG. 10F) huDF final time point post-scratch-RPRPDDLEI-loaded mEV's (SEQ ID NO: 1). Loaded mEV's exhibit higher wound healing potential than even free Cx43 CT peptides, confirming loading of mEV's with RPRPDDLEI (SEQ ID NO: 1).

FIG. 11A-11B—mEV's innately protect against ischemia-induced scar formation in a mouse model of IR injury. FIG. 11A displays analyzed results indicating ˜25% reduction in scar size following 100 uL oral gavage of milk-derived exosomes. FIG. 11B displays echo results for scar size in each group, visually confirming data displayed.

FIG. 12A-12D—RPRPDDLEI-loaded mEV's (SEQ ID NO: 1) exhibit high levels of radioprotection, equal or greater than 100 uM aCT1, a phase III clinical trial peptide for radioprotection. (FIG. 12A) Mitochondrial Tracker Assay (MTS) results for IEC-6 cells irradiated at 6 Gy, with cultures stopped after 72 hours. RPRPDDLEI Loaded mEV's (SEQ ID NO: 1) (Milacta) show equal or greater radioprotection (3rd bar from the Y axis) than aCT1 (bar furthest from the Y axis). The bar closest to the Y axis is vehicle control treated cells. The second bar from the Y axis are unloaded exosomes (FIG. 12B) Ethidium Homodimer-1 fluorescent intensity measurements for IEC-6 cells irradiated at 6 Gy, with cultures stopped after 72 hours. RPRPDDLEI Loaded mEV's (SEQ ID NO: 1) (Milacta) show equal or greater radioprotection (2nd bar from the Y axis) than aCT1 (1st bar from the Y axis), with mEV's naturally providing a statistical benefit as well (3^{rd} bar from the Y axis). Bar furthest from the Y axis showing greatest levels of cell death is the vehicle control. C&D) IEC-6 cells stained with Calcein AM (Green, as represented in greyscale) and Ethidium Homodimer-1 (red, as represented in greyscale), with FIG. 12C representing vehicle treated samples, and FIG. 12D representing Milacta treated groups. Imaging shows uncontrolled cell death emanating from a central point in vehicle groups, while Milacta treatment completely prevents radiation-induced Bystander effect-mediated cell death. Irradiation of isolated mEVs was found to leave the mEVs morphologically intact indicating that irradiation can be used as a method to sterilize mEVs or solutions containing mEVs such as mEV concentrates, whey or milk prior to administration or storage.

FIG. 13A-13B—Demonstrates that IgG is present on small EVs. (FIG. 13A) IgG Dynamic range as tested on nitrocellulose (1.0 second). (FIG. 13 B) Demonstrates IgG presence on bovine Milk Exosomes.

FIG. 14A-14B—Demonstrates that FcRN is present on Caco-2 cells. FIG. 14A shows. Cells stained with Hoechst stain (blue, as represented in greyscale) for nuclei and for FcRN (green, as represented in greyscale) FIG. 14B shows control ells (Hoechst stained+secondary antibody only).

FIG. 15—Experimental design for determining small EV uptake in Caco2 cells and IgG influence on uptake.

FIG. 16A-16F—Small EVs are taken up into Caco-2 cells. Caco2 Cells with (FIG. 16A) CTDR-sEVs after 30-minute incubation with no block. CTDR-sEVs visualized by red fluorescence. Nuclei visualized with blue fluorescence; (FIG. 16B) 488-IgG after 30-minute incubation. 488-IgG visualized with green fluorescence; (FIG. 16C) CTDR-sEVs with a protein G block after 30-minute incubation; (FIG. 16D) CTDR-sEVs co-administered simultaneously with 488-IgG and incubated for 30-minutes; (FIG. 16E) CTDR-sEVs for 30-minutes following a 30-minute incubation with 488-IgG; (FIG. 16F) CTDR-sEVs for 30-minutes following a 120-minute incubation with 488-IgG.

FIG. 17—Response of EV uptake to IgG pretreatment or direct protein G application.

FIG. 18—IgG-488 Uptake into Caco-2 cells.

FIG. 19—Blocking FcRN with unlabeled IgG as compared to 488-labeled IgG.

FIG. 20—IgG and EV colocalization (within circled region).

FIG. 21A-21D—Response to mEVs in scratch-wounded monolayers (FIG. 21B) of Human Dermal Fibroblasts and Madin-Darby Canine Kidney (MDCK) lines expressing high (MDCK Cx43-high) and low levels of Cx43 (MDCK Cx43-low). The results indicate that scratch-wounding (FIG. 21C) in all three cell lines resulted in significant (p<0.05) increases in mEV uptake over uninjured cells (FIG. 21D). FIG. 21A shows transmission electron microscopy of untreated and uninjured control mEVs.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and subrange is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlett, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

Definitions of common terms and techniques in chemistry and organic chemistry can be found in Smith. Organic Synthesis, published by Academic Press. 2016; Tinoco et al. Physical Chemistry, 5^{th} edition (2013) published by Pearson; Brown et al., Chemistry, The Central Science 14^{th} ed. (2017), published by Pearson, Clayden et al., Organic Chemistry, 2^{nd} ed. 2012, published by Oxford University Press; Carey and Sunberg, Advanced Organic Chemistry, Part A: Structure and Mechanisms, 5^{th} ed. 2008, published by Springer; Carey and Sunberg, Advanced Organic Chemistry, Part B: Reactions and Synthesis, 5^{th} ed. 2010, published by Springer, and Vollhardt and Schore, Organic Chemistry, Structure and Function; 8^{th} ed. (2018) published by W.H. Freeman.

Definitions of common terms, analysis, and techniques in genetics can be found in e.g., Hartl and Clark. Principles of Population Genetics. 4^{th} Ed. 2006, published by Oxford University Press. Published by Booker. Genetics: Analysis and Principles, 7^{th} Ed. 2021, published by McGraw Hill; Isik et al., Genetic Data Analysis for Plant and Animal Breeding. First ed. 2017. published by Springer International Publishing AG; Green, E. L. Genetics and Probability in Animal Breeding Experiments. 2014, published by Palgrave; Bourdon, R. M. Understanding Animal Breeding. 2000 2^{nd} Ed. published by Prentice Hall; Pal and Chakravarty. Genetics and Breeding for Disease Resistance of Livestock. First Ed. 2019, published by Academic Press; Fasso, D. Classification of Genetic Variance in Animals. First Ed. 2015, published by Callisto Reference; Megahed, M. Handbook of Animal Breeding and Genetics, 2013, published by Omniscriptum Gmbh & Co. Kg., LAP Lambert Academic Publishing; Reece. Analysis of Genes and Genomes. 2004, published by John Wiley & Sons. Inc; Deonier et al., Computational Genome Analysis. 5^{th} Ed. 2005, published by Springer-Verlag, New York; Meneely, P. Genetic Analysis: Genes, Genomes, and Networks in Eukaryotes. 3^{rd} Ed. 2020, published by Oxford University Press.

As used herein, the singular forms “a” “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g., a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, a “biological sample” refers to a sample obtained from, made by, secreted by, excreted by, or otherwise containing part of or from a biologic entity. A biologic sample can contain whole cells and/or live cells and/or cell debris, and/or cell products, and/or virus particles. The biological sample can contain (or be derived from) a “bodily fluid”. The biological sample can be obtained from an environment (e.g., water source, soil, air, and the like). Such samples are also referred to herein as environmental samples. As used herein “bodily fluid” refers to any non-solid excretion, secretion, or other fluid present in an organism and includes, without limitation unless otherwise specified or is apparent from the description herein, amniotic fluid, aqueous humor, vitreous humor, bile, blood or component thereof (e.g. plasma, serum, etc.), breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from an organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

As used herein “cancer” refers to one or more types of cancer including, but not limited to, acute lymphoblastic leukemia, acute myeloid leukemia, adrenocortical carcinoma, Kaposi Sarcoma, AIDS-related lymphoma, primary central nervous system (CNS) lymphoma, anal cancer, appendix cancer, astrocytomas, atypical teratoid/Rhabdoid tumors, basal cell carcinoma of the skin, bile duct cancer, bladder cancer, bone cancer (including but not limited to Ewing Sarcoma, osteosarcomas, and malignant fibrous histiocytoma), brain tumors, breast cancer, bronchial tumors, Burkitt lymphoma, carcinoid tumor, cardiac tumors, germ cell tumors, embryonal tumors, cervical cancer, cholangiocarcinoma, chordoma, chronic lymphocytic leukemia, chronic myelogenous leukemia, chronic myeloproliferative neoplasms, colorectal cancer, craniopharyngioma, cutaneous T-Cell lymphoma, ductal carcinoma in situ, endometrial cancer, ependymoma, esophageal cancer, esthesioneuroblastoma, extracranial germ cell tumor, extragonadal germ cell tumor, eye cancer (including, but not limited to, intraocular melanoma and retinoblastoma), fallopian tube cancer, gallbladder cancer, gastric cancer, gastrointestinal carcinoid tumor, gastrointestinal stromal tumors, central nervous system germ cell tumors, extracranial germ cell tumors, extragonadal germ cell tumors, ovarian germ cell tumors, testicular cancer, gestational trophoblastic disease, Hairy cell leukemia, head and neck cancers, hepatocellular (liver) cancer, Langerhans cell histiocytosis, Hodgkin lymphoma, hypopharyngeal cancer, islet cell tumors, pancreatic neuroendocrine tumors, kidney (renal cell) cancer, laryngeal cancer, leukemia, lip cancer, oral cancer, lung cancer (non-small cell and small cell), lymphoma, melanoma, Merkel cell carcinoma, mesothelioma, metastatic squamous cell neck cancer, midline tract carcinoma with and without NUT gene changes, multiple endocrine neoplasia syndromes, multiple myeloma, plasma cell neoplasms, mycosis fungoides, myelodysplastic syndromes, myelodysplastic/myeloproliferative neoplasms, chronic myelogenous leukemia, nasal cancer, sinus cancer, non-Hodgkin lymphoma, pancreatic cancer, paraganglioma, paranasal sinus cancer, parathyroid cancer, penile cancer, pharyngeal cancer, pheochromocytoma, pituitary cancer, peritoneal cancer, prostate cancer, rectal cancer, Rhabdomyosarcoma, salivary gland cancer, uterine sarcoma, Sezary syndrome, skin cancer, small intestine cancer, large intestine cancer (colon cancer), soft tissue sarcoma, T-cell lymphoma, throat cancer, oropharyngeal cancer, nasopharyngeal cancer, hypopharyngeal cancer, thymoma, thymic carcinoma, thyroid cancer, transitional cell cancer of the renal pelvis and ureter, urethral cancer, uterine cancer, vaginal cancer, cervical cancer, vascular tumors and cancer, vulvar cancer, and Wilms Tumor.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention. For example, in the appended claims, any of the claimed embodiments can be used in any combination.

As used herein, “administering” refers to any suitable administration for the agent(s) being delivered and/or subject receiving said agent(s) and can be oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration routes can be, for instance, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated, subject being treated, and/or agent(s) being administered.

As used herein, “agent” refers to any substance, compound, molecule, and the like, which can be administered to a subject on a subject to which it is administered to. An agent can be inert. An agent can be an active agent. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

As used herein, “anti-infective” refers to compounds or molecules that can either kill an infectious agent and/or modulate or inhibit its activity, infectivity, replication, and/or spreading such that its infectivity is reduced or eliminated and/or the disease or symptom thereof that it is associated is less severe or eliminated. Anti-infectives include, but are not limited to, antibiotics, antibacterials, antifungals, antivirals, and antiprotozoals.

The term “biocompatible”, as used herein, refers to a substance or object that performs its desired function when introduced into an organism without inducing significant inflammatory response, immunogenicity, or cytotoxicity to native cells, tissues, or organs, or to cells, tissues, or organs introduced with the substance or object. For example, a biocompatible product is a product that performs its desired function when introduced into an organism without inducing significant inflammatory response, immunogenicity, or cytotoxicity to native cells, tissues, or organs.

Biocompatibility, as used herein, can be quantified using the following in vivo biocompatibility assay. A material or product is considered biocompatible if it produces, in a test of biocompatibility related to immune system reaction, less than 50%, 45%, 40%, 35%, 30%, 25%, 20%, 15%, 10%, 8%, 6%, 5%, 4%, 3%, 2%, or 1% of the reaction, in the same test of biocompatibility, produced by a material or product the same as the test material or product except for a lack of the surface modification on the test material or product. Examples of useful biocompatibility tests include measuring and assessing cytotoxicity in cell culture, inflammatory response after implantation (such as by fluorescence detection of cathepsin activity), and immune system cells recruited to implant (for example, macrophages and neutrophils).

As used herein, “biologic agent” refers to any compound, composition, molecule and the like that is made by a living organism and include, without limitation, polynucleotides (e.g., DNA, RNA), peptides and polypeptides, and chemical compounds (e.g., hormones, chemokines, and cytokines).

As used herein, “chemotherapeutic agent” or “chemotherapeutic” refers to a therapeutic agent utilized to prevent or treat cancer.

As used herein, “control” refers to an alternative subject or sample used in an experiment for comparison purpose and included to minimize or distinguish the effect of variables other than an independent variable.

As used herein, the terms “disease” or “disorder” are used interchangeably throughout this specification and refer to any alternation in state of the body or of some of the organs, interrupting or disturbing the performance of the functions and/or causing symptoms such as discomfort, dysfunction, distress, or even death to the person afflicted or those in contact with a person. A disease or disorder can also be related to a distemper, ailing, ailment, malady, disorder, sickness, illness, complaint, indisposition, or affliction.

As used herein, “dose,” “unit dose,” or “dosage” can refer to physically discrete units suitable for use in a subject, each unit containing a predetermined quantity of the primary and/or a secondary active agent calculated to produce the desired response or responses in association with its administration.

As used herein, “immunomodulator,” refers to an agent, such as a therapeutic agent, which is capable of modulating or regulating one or more immune function or response.

As used herein, “infection” as used herein refers to presence of an infective agent, such as a pathogen, e.g., a microorganism, in or on a subject, which, if its presence or growth were inhibited, would result in a benefit to the subject. Hence, the term refers to the state produced by the establishment, more particularly invasion and multiplication, of an infective agent, such as a pathogen, e.g., a microorganism, in or on a suitable host. An infection may produce tissue injury and progress to overt disease through a variety of cellular and toxic mechanisms.

As used herein, “inflammation” generally refers to a response in vasculated tissues to cellular or tissue injury usually caused by physical, chemical and/or biological agents, that is marked in the acute form by the classical sequences of pain, heat, redness, swelling, and loss of function, and serves as a mechanism initiating the elimination, dilution or walling-off of noxious agents and/or of damaged tissue. Inflammation histologically involves a complex series of events, including dilation of the arterioles, capillaries, and venules with increased permeability and blood flow, exudation of fluids including plasma proteins, and leukocyte migration into the inflammatory focus. Further, the term encompasses inflammation caused by extraneous physical or chemical injury or by biological agents, e.g., viruses, bacteria, fungi, protozoan or metazoan parasite infections, as well as inflammation which is seemingly unprovoked, e.g., which occurs in the absence of demonstrable injury or infection, inflammation responses to self-antigens (auto-immune inflammation), inflammation responses to engrafted xenogeneic or allogeneic cells, tissues or organs, inflammation responses to allergens, etc. The term covers both acute inflammation and chronic inflammation. Also, the term includes both local or localised inflammation, as well as systemic inflammation, i.e., where one or more inflammatory processes are not confined to a particular tissue but occur generally in the endothelium and/or other organ systems.

As used herein, “isolated” means separated from constituents, cellular and otherwise, in which the polynucleotide, peptide, polypeptide, protein, antibody, or fragments thereof, are normally associated with in nature. A non-naturally occurring polynucleotide, peptide, polypeptide, protein, antibody, or fragments thereof, do not require “isolation” to distinguish it from its naturally occurring counterpart.

The term “molecular weight”, as used herein, generally refers to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (MW) as opposed to the number-average molecular weight (Mₙ). Capillary viscometry provides estimates of molecular weight as the inherent viscosity determined from a dilute polymer solution using a particular set of concentration, temperature, and solvent conditions.

As used herein, “non-human mammal” refers to any mammal that is not a human.

As used herein, “nucleic acid,” “nucleotide sequence,” and “polynucleotide” can be used interchangeably herein and can generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide as used herein can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide”, “nucleotide sequences” and “nucleic acids” also includes PNAs (peptide nucleic acids), phosphonothioates, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompasses a nucleic acid and polynucleotide as defined elsewhere herein.

As used herein, the term “recombinant” or “engineered” can generally refer to a non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter), etc. Recombinant or engineered can also refer to the polypeptide encoded by the recombinant nucleic acid. Non-naturally occurring nucleic acids or polypeptides include nucleic acids and polypeptides modified by man.

As used herein, the term “specific binding” refers to non-covalent physical association of a first and a second moiety wherein the association between the first and second moieties is at least 2 times as strong, at least 5 times as strong as, at least 10 times as strong as, at least 50 times as strong as, at least 100 times as strong as, or stronger than the association of either moiety with most or all other moieties present in the environment in which binding occurs. Binding of two or more entities may be considered specific if the equilibrium dissociation constant, Kd, is 10⁻³ M or less, 10⁻⁴ M or less, 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a Kd of greater than 10⁻³ M). In some embodiments, specific binding, which can be referred to as “molecular recognition,” is a saturable binding interaction between two entities that is dependent on complementary orientation of functional groups on each entity. Examples of specific binding interactions include primer-polynucleotide interaction, aptamer-aptamer target interactions, antibody-antigen interactions, avidin-biotin interactions, ligand-receptor interactions, metal-chelate interactions, hybridization between complementary nucleic acids, etc.

As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory or CD-ROM or on a server that can be accessed by a user via, e.g., a web interface.

As used herein, “therapeutic” refers to treating, healing, and/or ameliorating a disease, disorder, condition, or side effect, or to decreasing in the rate of advancement of a disease, disorder, condition, or side effect. A “therapeutically effective amount” can therefore refer to an amount of a compound that can yield a therapeutic effect.

As used herein, the terms “treating”, and “treatment” can refer generally to obtaining a desired pharmacological and/or physiological effect. The effect can be, but does not necessarily have to be, prophylactic in terms of preventing or partially preventing a disease, symptom or condition thereof, such as cancer, inflammatory disease or disorder, and/or a mechanical or non-mechanical injury. The effect can be therapeutic in terms of a partial or complete cure of a disease, condition, symptom or adverse effect attributed to the disease, disorder, or condition. The term “treatment” as used herein covers any treatment of cancer, inflammatory disease or disorder, and/or a mechanical or non-mechanical injury, in a subject, particularly a human or non-human animal, such as a non-human mammal, and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

As used herein, the terms “weight percent,” “wt %,” and “wt. %,” which can be used interchangeably, indicate the percent by weight of a given component based on the total weight of a composition of which it is a component, unless otherwise specified. That is, unless otherwise specified, all wt % values are based on the total weight of the composition. It should be understood that the sum of wt % values for all components in a disclosed composition or formulation are equal to 100. Alternatively, if the wt % value is based on the total weight of a subset of components in a composition, it should be understood that the sum of wt % values the specified components in the disclosed composition or formulation are equal to 100.

All publications, published patent documents, and patent applications cited herein are hereby incorporated by reference to the same extent as though each individual publication, published patent document, or patent application was specifically and individually indicated as being incorporated by reference.

### Overview

Oral delivery of therapeutics is desirable but, in many cases, is not achievable with conventional delivery approaches for some therapeutics. For example, first and/or second pass metabolism reduces bioavailability of the therapeutics such that oral delivery is not clinically viable. In other cases, the therapeutics are liable or sensitive to different environments such as acidic pH. Further, fragile drugs such as insulin or heparin are primarily administered via injection so as to ensure sufficient bioavailability. As such, there exists a need for improved delivery approaches for therapeutics, particularly oral delivery.

With that said, embodiments herein disclosed herein can provide milk exosomes, including bovine milk exosomes, that can be capable of targeting injury sites (e.g., mechanical or non-mechanical injury sites) and/or cancer cells and their use to treat an injury or cancer. In some embodiments, the milk exosomes can be loaded with an exogenous cargo. In some embodiments, the milk exosomes are enriched for Connexin 43 positive exosomes. In some embodiments, treatment includes co-administration of one or more agents that increase ATP production and/or release. In some embodiments, treatment includes co-administration or pretreatment with IgG. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Targeted Milk Exosomes

Described in certain example embodiments are milk exosomes comprising an exogenous cargo, where the milk exosome is capable of targeting an injury site or a cancer cell or cell population by targeting or stimulating adenosine triphosphate (ATP) secretion and/or concentration and/or neonatal Fc receptor (FcRN). Without being bound by theory, ATP can be concentrated in certain areas and/or in or near certain cells, such as cells to be targeted (also referred to herein as “target” cells). For example, areas or cells injured by mechanical or non-mechanical injury or cancer cells and/or tumors, or areas of inflammation can have greater concentrations of secreted ATP. The milk exosome of the present disclosure can home or target these areas (e.g., injury areas or cell microenvironments) and/or cells having increased ATP concentration. As demonstrated in the Working Examples herein, it is demonstrated that the milk exosomes are capable of homing to the regions and/or cells having a higher concentration of ATP. Further, and without being bound by theory, by stimulating cells to produce and/or release ATP this can act to home or signal the milk exosomes of the present disclosure to target those cells and/or areas (including cells in those areas). As used in this context, “concentrated” refers to a region that has a greater amount or number of molecules of ATP per unit of area or volume as compared to another area or volume. A concentration gradient may be formed where the area closest to the target cell or cells or region is greatest and is decreased as the distance from the target cell or cells or region is increased. In some embodiments, “concentrated” in this context can refer to 0.001 to 1,000 or more percent greater amount or number of molecules of ATP per unit of a first area or volume as compared to a second area or volume. Additionally, as demonstrated in the Working Examples, immunoglobulins present in the milk exosome can interact with and/or bind a FcRN on a cell, which can increase uptake of the milk exosome in those cells. In some embodiments, the milk exosomes are bovine milk exosomes, ovine milk exosomes, camelid milk exosomes, and/or human milk exosomes.

In certain example embodiments, the injury site is a mechanical injury or a non-mechanical injury. In certain example embodiments, the non-mechanical injury is a chemical injury, electrical injury, radiation injury, an ischemic injury (e.g., an injury caused by or resulting from an ischemic event) or an injury resulting from inflammatory processes or inflammation (also referred to herein as an “inflammatory injury”). In some embodiments the mechanical injury is a wound (inflicted or generated by any process), burn, etc. Other exemplary injuries are described in greater detail elsewhere herein.

In some embodiments, the milk exosomes can target a cancer cell or cells a region or cells subject to or affected by a viral infection, a bacterial infection, a parasite infection, a mechanical injury (e.g., external and internal wounds and tissue injuries, cuts, scrapes, burns, surgical wounds, wounds secondary to infection, disease, or condition, etc.), cancer, non-mechanical injury (e.g., chemical injury, electrical injury, radiation injury, inflammatory injury, ischemic and/or hypoxic injuries (e.g. myocardial infarction, ischemic wounds and/or stroke), multiple sclerosis, psoriasis, scleroderma, acne, eczema, or a disease of the skin and/or connective tissues, cardiac diseases or disorders, neurodegenerative diseases or disorders, neurological disorders, atherosclerosis, pathologies involving epithelial permeabilization and/or neovascularization (e.g., angiogenesis or vasculogenesis), respiratory distress syndrome (RDS), reperfusion injuries, dermal vascular blemish or malformation, macular degeneration, neovascularization of choriocapillaris through Bruch's membrane, diabetic retinopathy, (inflammatory and inflammation-related diseases and disorders), and radiation dermatitis.

Wounds can be chronic wounds or wounds that appear to not completely heal. Wounds that have not healed within three months, for example, are said to be chronic. Chronic wounds include, diabetic foot ulcers, ischemic, venous ulcers, venous leg ulcers, venous stasis, arterial, pressure, vasculitic, infectious, decubitis, burn, trauma-induced, gangrenous and mixed ulcers. Chronic wounds include wounds that are characterized by and/or chronic inflammation, deficient and overprofuse granulation tissue differentiation and failure of re-epithelialization and wound closure and longer repair times. Chronic wounds can include ocular ulcers, including corneal ulcers. Use of the disclosed invention in wound healing and tissue regeneration can include in humans and agricultural, sports and pet animals.

Tissue injuries can result from, for example, a cut, scrape, compression wound, stretch injury, laceration wound, crush wound, bite wound, graze, bullet wound, explosion injury, body piercing, stab wound, surgical wound, surgical intervention, medical intervention, host rejection following cell, tissue or organ grafting, pharmaceutical effect, pharmaceutical side-effect, bed sore, radiation injury, radiation illness, cosmetic skin wound, inflammatory injury, internal organ injury, disease process (e.g., asthma, cancer), infection, infectious agent, developmental process, maturational process (e.g., acne), genetic abnormality, developmental abnormality, environmental toxin, allergen, scalp injury, facial injury, jaw injury, sex organ injury, joint injury, excretory organ injury, foot injury, finger injury, toe injury, bone injury, eye injury, corneal injury, muscle injury, adipose tissue injury, lung injury, airway injury, hernia, anus injury, piles, ear injury, skin injury, abdominal injury, retinal injury, eye injury, corneal injury, arm injury, leg injury, athletic injury, back injury, birth injury, premature birth injury, toxic bite, sting, injury to barrier function, injury to endothelial barrier function, injury to epithelial barrier function, tendon injury, ligament injury, heart injury, heart valve injury, vascular system injury, cartilage injury, lymphatic system injury, craniocerebral trauma, dislocation, esophageal perforation, fistula, nail injury, foreign body, fracture, frostbite, hand injury, heat stress disorder, laceration, neck injury, self-mutilation, shock, traumatic soft tissue injury, spinal cord injury, spinal injury, sprain, strain, tendon injury, ligament injury, cartilage injury, thoracic injury, tooth injury, trauma, nervous system injury, burn, burn wound, wind burn, sun burn, chemical burn, aging, aneurism, stroke, surgical radiation injury, digestive tract injury, infarct, or ischemic injury.

Cardiac diseases and disorders that can yield injured cell or cells or otherwise be targeted by the milk exosomes of the present disclosure can include, but are not limited to, myocardial infarction, cardio myopathies (e.g., hypertrophic cardiomyopathy), arrhythmias, congestive heart failure. The regenerative effects of the provided composition may result in beneficial changes in membrane excitability and ion transients of the heart. There are many different types of arrhythmia that can lead to abnormal function in the human heart. Arrhythmias include, but are not limited to bradycardias, tachycardias, alternans, automaticity defects, reentrant arrhythmias, fibrillation, AV nodal arrhythmias, atrial arrhythmias and triggered beats, Long QT syndrome, Short QT syndrome, Brugada syndrome, premature atrial Contractions, wandering Atrial pacemaker, Multifocal atrial tachycardia, Atrial flutter, Atrial fibrillation, Supraventricular tachycardia, AV nodal reentrant tachycardia is the most common cause of Paroxysmal Supraventricular Tachycardia, Junctional rhythm, Junctional tachycardia, Premature junctional complex, Wolff-Parkinson—White syndrome, Lown-Ganong-Levine syndrome, Premature Ventricular Contractions (PVC) sometimes called Ventricular Extra Beats, alternans and discordant alternans, Accelerated idioventricular rhythm, Monomorphic Ventricular tachycardia, Polymorphic ventricular tachycardia, Ventricular fibrillation, First degree heart block, which manifests as PR prolongation, Second degree heart block, Type 1 Second degree heart block, Type 2 Second degree heart block, Third degree heart block, and several accessory pathway disorders (e.g., Wolff-Parkinson—White syndrome (WPW)).

Neurodegenerative and neurological disorders that can yield injured cell or cells or otherwise be targeted by the milk exosomes of the present disclosure can include, but are not limited to dementia, Alzheimer's disease, Parkinson's disease and related PD-diseases, amyotrophic lateral sclerosis (ALS), motor neuron disease, schizophrenia, spinocerebellar ataxia, prion disease, Spinal muscular atrophy (SMA), multiple sclerosis, epilepsy and other seizure disorders, and Huntington's disease.

Inflammatory diseases and inflammatory-related diseases and disorders that can lead to injured cells or otherwise be targeted by the milk exosomes of the present disclosure can be asthma, eczema, sinusitis, atherosclerosis, arthritis (including but not limited to rheumatoid arthritis), inflammatory bowel disease, cutaneous and systemic mastocytosis, psoriasis, and multiple sclerosis. As used herein, the term “inflammatory disorder” can include diseases or disorders which are caused, at least in part, or exacerbated, by inflammation, which is generally characterized by increased blood flow, edema, activation of immune cells (e.g., proliferation, cytokine production, or enhanced phagocytosis), heat, redness, swelling, pain and/or loss of function in the affected tissue or organ. The cause of inflammation can be due to physical damage, chemical substances, micro-organisms, tissue necrosis, cancer, or other agents or conditions. Inflammatory disorders include acute inflammatory disorders, chronic inflammatory disorders, and recurrent inflammatory disorders. Acute inflammatory disorders are generally of relatively short duration, and last for from about a few minutes to about one to two days, although they can last several weeks. Characteristics of acute inflammatory disorders include increased blood flow, exudation of fluid and plasma proteins (edema) and emigration of leukocytes, such as neutrophils. Chronic inflammatory disorders, generally, are of longer duration, e.g., weeks to months to years or longer, and are associated histologically with the presence of lymphocytes and macrophages and with proliferation of blood vessels and connective tissue. Recurrent inflammatory disorders include disorders which recur after a period of time, or which have periodic episodes. Some inflammatory disorders fall within one or more categories. Exemplary inflammatory disorders include but are not limited to atherosclerosis; arthritis; inflammation-promoted cancers; asthma; autoimmune uveitis; adoptive immune response; dermatitis; multiple sclerosis; diabetic complications; osteoporosis; Alzheimer's disease; cerebral malaria; hemorrhagic fever; autoimmune disorders; and inflammatory bowel disease. In some embodiments, the inflammatory disorder is an autoimmune disorder that, in some embodiments, is selected from lupus, rheumatoid arthritis, and autoimmune encephalomyelitis. In some embodiments, the inflammatory disorder is a brain-related inflammatory disorder. The term “brain-related inflammatory” disorder is used herein to refer to a subset of inflammatory disorders that are caused, at least in part, or originate or are exacerbated, by inflammation in the brain of a subject.

In certain example embodiments, the milk exosome does not contain an exogenous targeting moiety. In other words, in some embodiments, the milk exosome does not contain a non-native targeting moiety.

In certain example embodiments, the injury or site thereof has a greater concentration of ATP than a non-injury or site thereof. In certain example embodiments, the cancer cell or cancer cell population has or has a microenvironment that has a greater concentration of ATP than a microenvironment of a non-cancer cell or non-cancer cell population. In some embodiments, the concentration of ATP of the injury or at the site thereof or in or at the cancer cell or cancer cell population or microenvironment thereof is 0.01%, to/or 0.02%, 0.03%, 0.04%, 0.05%, 0.06%, 0.07%, 0.08%, 0.09%, 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.31%, 0.32%, 0.33%, 0.34%, 0.35%, 0.36%, 0.37%, 0.38%, 0.39%, 0.4%, 0.41%, 0.42%, 0.43%, 0.44%, 0.45%, 0.46%, 0.47%, 0.48%, 0.49%, 0.5%, 0.51%, 0.52%, 0.53%, 0.54%, 0.55%, 0.56%, 0.57%, 0.58%, 0.59%, 0.6%, 0.61%, 0.62%, 0.63%, 0.64%, 0.65%, 0.66%, 0.67%, 0.68%, 0.69%, 0.7%, 0.71%, 0.72%, 0.73%, 0.74%, 0.75%, 0.76%, 0.77%, 0.78%, 0.79%, 0.8%, 0.81%, 0.82%, 0.83%, 0.84%, 0.85%, 0.86%, 0.87%, 0.88%, 0.89%, 0.9%, 0.91%, 0.92%, 0.93%, 0.94%, 0.95%, 0.96%, 0.97%, 0.98%, 0.99%1, 1%, 2%, 3%, 4%, 5%, 6%, 7%, 8%, 9%, 1%, 110%, 2%, 3%, %14%, %15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 101%, 102%, 103%, 104%, 105%, 106%, 107%, 108%, 109%, 110%, 111%, 112%, 113%, 114%, 115%, 116%, 117%, 118%, 119%, 120%, 121%, 122%, 123%, 124%, 125%, 126%, 127%, 128%, 129%, 130%, 131%, 132%, 133%, 134%, 135%, 136%, 137%, 138%, 139%, 140%, 141%, 142%, 143%, 144%, 145%, 146%, 147%, 148%, 149%, 150%, 151%, 152%, 153%, 154%, 155%, 156%, 157%, 158%, 159%, 160%, 161%, 162%, 163%, 164%, 165%, 166%, 167%, 168%, 169%, 170%, 171%, 172%, 173%, 174%, 175%, 176%, 177%, 178%, 179%, 180%, 181%, 182%, 183%, 184%, 185%, 186%, 187%, 188%, 189%, 190%, 191%, 192%, 193%, 194%, 195%, 196%, 197%, 198%, 199%, 200%, 201%, 202%, 203%, 204%, 205%, 206%, 207%, 208%, 209%, 210%, 211%, 212%, 213%, 214%, 215%, 216%, 217%, 218%, 219%, 220%, 221%, 222%, 223%, 224%, 225%, 226%, 227%, 228%, 229%, 230%, 231%, 232%, 233%, 234%, 235%, 236%, 237%, 238%, 239%, 240%, 241%, 242%, 243%, 244%, 245%, 246%, 247%, 248%, 249%, 250%, 251%, 252%, 253%, 254%, 255%, 256%, 257%, 258%, 259%, 260%, 261%, 262%, 263%, 264%, 265%, 266%, 267%, 268%, 269%, 270%, 271%, 272%, 273%, 274%, 275%, 276%, 277%, 278%, 279%, 280%, 281%, 282%, 283%, 284%, 285%, 286%, 287%, 288%, 289%, 290%, 291%, 292%, 293%, 294%, 295%, 296%, 297%, 298%, 299%, 300%, 301%, 302%, 303%, 304%, 305%, 306%, 307%, 308%, 309%, 310%, 311%, 312%, 313%, 314%, 315%, 316%, 317%, 318%, 319%, 320%, 321%, 322%, 323%, 324%, 325%, 326%, 327%, 328%, 329%, 330%, 331%, 332%, 333%, 334%, 335%, 336%, 337%, 338%, 339%, 340%, 341%, 342%, 343%, 344%, 345%, 346%, 347%, 348%, 349%, 350%, 351%, 352%, 353%, 354%, 355%, 356%, 357%, 358%, 359%, 360%, 361%, 362%, 363%, 364%, 365%, 366%, 367%, 368%, 369%, 370%, 371%, 372%, 373%, 374%, 375%, 376%, 377%, 378%, 379%, 380%, 381%, 382%, 383%, 384%, 385%, 386%, 387%, 388%, 389%, 390%, 391%, 392%, 393%, 394%, 395%, 396%, 397%, 398%, 399%, 400%, 401%, 402%, 403%, 404%, 405%, 406%, 407%, 408%, 409%, 410%, 411%, 412%, 413%, 414%, 415%, 416%, 417%, 418%, 419%, 420%, 421%, 422%, 423%, 424%, 425%, 426%, 427%, 428%, 429%, 430%, 431%, 432%, 433%, 434%, 435%, 436%, 437%, 438%, 439%, 440%, 441%, 442%, 443%, 444%, 445%, 446%, 447%, 448%, 449%, 450%, 451%, 452%, 453%, 454%, 455%, 456%, 457%, 458%, 459%, 460%, 461%, 462%, 463%, 464%, 465%, 466%, 467%, 468%, 469%, 470%, 471%, 472%, 473%, 474%, 475%, 476%, 477%, 478%, 479%, 480%, 481%, 482%, 483%, 484%, 485%, 486%, 487%, 488%, 489%, 490%, 491%, 492%, 493%, 494%, 495%, 496%, 497%, 498%, 499%, 500%, 501%, 502%, 503%, 504%, 505%, 506%, 507%, 508%, 509%, 510%, 511%, 512%, 513%, 514%, 515%, 516%, 517%, 518%, 519%, 520%, 521%, 522%, 523%, 524%, 525%, 526%, 527%, 528%, 529%, 530%, 531%, 532%, 533%, 534%, 535%, 536%, 537%, 538%, 539%, 540%, 541%, 542%, 543%, 544%, 545%, 546%, 547%, 548%, 549%, 550%, 551%, 552%, 553%, 554%, 555%, 556%, 557%, 558%, 559%, 560%, 561%, 562%, 563%, 564%, 565%, 566%, 567%, 568%, 569%, 570%, 571%, 572%, 573%, 574%, 575%, 576%, 577%, 578%, 579%, 580%, 581%, 582%, 583%, 584%, 585%, 586%, 587%, 588%, 589%, 590%, 591%, 592%, 593%, 594%, 595%, 596%, 597%, 598%, 599%, 600%, 601%, 602%, 603%, 604%, 605%, 606%, 607%, 608%, 609%, 610%, 611%, 612%, 613%, 614%, 615%, 616%, 617%, 618%, 619%, 620%, 621%, 622%, 623%, 624%, 625%, 626%, 627%, 628%, 629%, 630%, 631%, 632%, 633%, 634%, 635%, 636%, 637%, 638%, 639%, 640%, 641%, 642%, 643%, 644%, 645%, 646%, 647%, 648%, 649%, 650%, 651%, 652%, 653%, 654%, 655%, 656%, 657%, 658%, 659%, 660%, 661%, 662%, 663%, 664%, 665%, 666%, 667%, 668%, 669%, 670%, 671%, 672%, 673%, 674%, 675%, 676%, 677%, 678%, 679%, 680%, 681%, 682%, 683%, 684%, 685%, 686%, 687%, 688%, 689%, 690%, 691%, 692%, 693%, 694%, 695%, 696%, 697%, 698%, 699%, 700%, 701%, 702%, 703%, 704%, 705%, 706%, 707%, 708%, 709%, 710%, 711%, 712%, 713%, 714%, 715%, 716%, 717%, 718%, 719%, 720%, 721%, 722%, 723%, 724%, 725%, 726%, 727%, 728%, 729%, 730%, 731%, 732%, 733%, 734%, 735%, 736%, 737%, 738%, 739%, 740%, 741%, 742%, 743%, 744%, 745%, 746%, 747%, 748%, 749%, 750%, 751%, 752%, 753%, 754%, 755%, 756%, 757%, 758%, 759%, 760%, 761%, 762%, 763%, 764%, 765%, 766%, 767%, 768%, 769%, 770%, 771%, 772%, 773%, 774%, 775%, 776%, 777%, 778%, 779%, 780%, 781%, 782%, 783%, 784%, 785%, 786%, 787%, 788%, 789%, 790%, 791%, 792%, 793%, 794%, 795%, 796%, 797%, 798%, 799%, 800%, 801%, 802%, 803%, 804%, 805%, 806%, 807%, 808%, 809%, 810%, 811%, 812%, 813%, 814%, 815%, 816%, 817%, 818%, 819%, 820%, 821%, 822%, 823%, 824%, 825%, 826%, 827%, 828%, 829%, 830%, 831%, 832%, 833%, 834%, 835%, 836%, 837%, 838%, 839%, 840%, 841%, 842%, 843%, 844%, 845%, 846%, 847%, 848%, 849%, 850%, 851%, 852%, 853%, 854%, 855%, 856%, 857%, 858%, 859%, 860%, 861%, 862%, 863%, 864%, 865%, 866%, 867%, 868%, 869%, 870%, 871%, 872%, 873%, 874%, 875%, 876%, 877%, 878%, 879%, 880%, 881%, 882%, 883%, 884%, 885%, 886%, 887%, 888%, 889%, 890%, 891%, 892%, 893%, 894%, 895%, 896%, 897%, 898%, 899%, 900%, 901%, 902%, 903%, 904%, 905%, 906%, 907%, 908%, 909%, 910%, 911%, 912%, 913%, 914%, 915%, 916%, 917%, 918%, 919%, 920%, 921%, 922%, 923%, 924%, 925%, 926%, 927%, 928%, 929%, 930%, 931%, 932%, 933%, 934%, 935%, 936%, 937%, 938%, 939%, 940%, 941%, 942%, 943%, 944%, 945%, 946%, 947%, 948%, 949%, 950%, 951%, 952%, 953%, 954%, 955%, 956%, 957%, 958%, 959%, 960%, 961%, 962%, 963%, 964%, 965%, 966%, 967%, 968%, 969%, 970%, 971%, 972%, 973%, 974%, 975%, 976%, 977%, 978%, 979%, 980%, 981%, 982%, 983%, 984%, 985%, 986%, 987%, 988%, 989%, 990%, 991%, 992%, 993%, 994%, 995%, 996%, 997%, 998%, 999%, 1000% greater or more than a non-injury or site thereof or a non-cancer cell or non-cancer cell population or microenvironment thereof

In certain example embodiments, the cancer cell or cancer cell population secretes ATP. In certain example embodiments the injured cell or cells secrete ATP.

In certain example embodiments, the milk exosome comprises connexin 43. In certain example embodiments, the connexin 43 is a native connexin 43 or wherein the connexin 43 is an engineered connexin 43. In some embodiments, the engineered connexin 43 is as described in WO 2020/028439.

In certain example embodiments, the exogenous cargo is a biologic molecule. In certain example embodiments, the biologic molecule is a lipid, polypeptide, peptide, a nucleic acid or a cellular metabolite. In certain example embodiments, the exogenous cargo is an ACT1 peptide, ACT11 peptide, an ACT minus I peptide, a selectide, or any combination thereof. In certain example embodiments, the exogenous cargo is any of those cargos set forth in WO 2020/028439, an ACT1 or ACT11 peptide, an ACT minus peptide (see e.g., ACT minus peptides set forth in WO 2022/076932), Gap19, JM2 (see e.g., U.S. Pat. No. 9,345,744 B2), a selectide (see e.g., WO 2022/076932), or any combination thereof. In certain example embodiments, the exogenous cargo is a peptide, including but not limited to an ACT-11 peptide. In certain example embodiments, the exogenous cargo is a peptide, including but not limited to an ACT-11-minus I peptide. Other peptide cargos include those set forth in International Patent Application Publication WO2020/028439, particularly at pages 67-82 and 85 and 106-111. In some embodiments, the cargo compound is esterified, such as described in International Patent Application Publication WO2020/028439 at page 81-86. In some embodiments, the cargo compound has multiple esterifications, such as described in International Patent Application Publication WO2020/028439 at page 81-86.

In certain example embodiments, the milk exosome, prior to containing the exogenous cargo, is isolated according to a method comprising (a) centrifuging a mammalian milk under conditions suitable to separate fats from one or more other components of the mammalian milk; (b) removing the separated fats from the mammalian milk; (c) after step (b) centrifuging the remaining mammalian milk one or more times and skimming any noticeable separated fats after each centrifuging in step (c); (d) filtering the remaining biological fluid after step (c); (e) optionally performing one or more ultracentrifugation steps after (d); (f) chelating divalent cations with about 10 mM to about 100 mM EDTA at about 30-42 degrees Celsius after (d) or optionally (e) and optionally for about 15-120 minutes; and (g) after (f), optionally performing tangential flow filtration to obtain a retentate, wherein the retentate is optionally ultracentrifuged via one or more ultracentrifugation steps or stored at −80 degrees C., and separating out fractions of the retentate, optionally via column separation, after the retentate is optionally ultracentrifuged or stored at −80 degrees C., wherein the method comprises step (e) or step (g) but not both. Additional methods of milk exosome isolation is described elsewhere herein.

Described in certain example embodiments here are populations of milk exosome comprising one or more milk exosomes of the present disclosure described herein. In certain example embodiments, the population is enriched for connexin 43 positive milk exosomes. In certain example embodiments, the milk exosome, prior to containing the exogenous cargo, is isolated according to a method comprising (a) centrifuging a mammalian milk under conditions suitable to separate fats from one or more other components of the mammalian milk; (b) removing the separated fats from the mammalian milk; (c) after step (b) centrifuging the remaining mammalian milk one or more times and skimming any noticeable separated fats after each centrifuging in step (c); (d) filtering the remaining biological fluid after step (c); (e) optionally performing one or more ultracentrifugation steps after (d); (f) chelating divalent cations with about 10 mM to about 100 mM EDTA at about 30-42 degrees Celsius after (d) or optionally (e) and optionally for about 15-120 minutes; and (g) after (f), optionally performing tangential flow filtration to obtain a retentate, wherein the retentate is optionally ultracentrifuged via one or more ultracentrifugation steps or stored at −80 degrees C., and separating out fractions of the retentate, optionally via column separation, after the retentate is optionally ultracentrifuged or stored at −80 degrees C., wherein the method comprises step (e) or step (g) but not both. In certain example embodiments, the method further comprises enriching the milk exosomes for connexin 43 expressing milk exosomes. In certain example embodiments, the milk exosomes do not contain an exogenous targeting moiety. In certain example embodiments, the exogenous cargo is a biologic molecule. In certain example embodiments, the biologic molecule is a polypeptide, peptide, or a nucleic acid. In certain example embodiments, the exogenous cargo is an ACT1 peptide, ACT11 peptide, an ACT minus peptide, a selectide, or any combination thereof.

In certain example embodiments, the milk exosome(s) or solutions containing the same (e.g., milk, whey, etc.) are sterilized and/or irradiated. In some embodiments the milk exosomes are sterilized. In some embodiments, the milk exosomes are sterilized by irradiation. In some embodiments, irradiation does not substantially disrupt the morphology of the milk exosome(s) yet sterilizes the milk exosome(s). In other words, irradiation leaves the milk exosome(s) morphologically intact and thus provides a method that is effective for sterilization of the milk exosome(s) and/or solutions or formulations containing the milk exosome(s). In some embodiments, milk exosomes(s) or formulation or solution containing the same are sterilized and/or irradiated prior to exosome isolation and/or storage.

In some embodiments, the milk exosomes have increased uptake in cells that have concentrated ATP or are in tissue or cell microenvironments with concentrated and/or secreted ATP as compared to cells that do not have concentrated levels of ATP or are in environments without concentrated ATP. In some embodiments, the milk exosomes have increased uptake in cells having FcRN on their surface. gastrointestinal cell, a heart cell, a liver cell, a brain cell, a nerve cell, reproductive cell, lung cell, a cancer cell, or any combination thereof. In some embodiments, the cell is a polar cell, meaning that the cells have a different apical and basal membrane surface. In some embodiments, the cell is an enterocyte. In some embodiments, the cells is a blood-brain barrier cell. In some embodiments, uptake is increased 0.01%, to/or 0.02%, 0.03%, 0.04%, 0.05%, 0.06%, 0.07%, 0.08%, 0.09%, 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.31%, 0.32%, 0.33%, 0.34%, 0.35%, 0.36%, 0.37%, 0.38%, 0.39%, 0.4%, 0.41%, 0.42%, 0.43%, 0.44%, 0.45%, 0.46%, 0.47%, 0.48%, 0.49%, 0.5%, 0.51%, 0.52%, 0.53%, 0.54%, 0.55%, 0.56%, 0.57%, 0.58%, 0.59%, 0.6%, 0.61%, 0.62%, 0.63%, 0.64%, 0.65%, 0.66%, 0.67%, 0.68%, 0.69%, 0.7%, 0.71%, 0.72%, 0.73%, 0.74%, 0.75%, 0.76%, 0.77%, 0.78%, 0.79%, 0.8%, 0.81%, 0.82%, 0.83%, 0.84%, 0.85%, 0.86%, 0.87%, 0.88%, 0.89%, 0.9%, 0.91%, 0.92%, 0.93%, 0.94%, 0.95%, 0.96%, 0.97%, 0.98%, 0.99%1, 1%, 2%, 3%, 4%, 5%, 6%, 7%, 8%, 9% 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 101%, 102%, 103%, 104%, 105%, 106%, 107%, 108%, 109%, 110%, 111%, 112%, 113%, 114%, 115%, 116%, 117%, 118%, 119%, 120%, 121%, 122%, 123%, 124%, 125%, 126%, 127%, 128%, 129%, 130%, 131%, 132%, 133%, 134%, 135%, 136%, 137%, 138%, 139%, 140%, 141%, 142%, 1430%, 144%, 145%, 146%, 147%, 148%, 149%, 150%, 151%, 152%, 153%, 154%, 155%, 156%, 157%, 158%, 159%, 160%, 161%, 162%, 163%, 164%, 165%, 166%, 167%, 168%, 169%, 170%, 171%, 172%, 173%, 174%, 175%, 176%, 177%, 178%, 179%, 180%, 181%, 182%, 183%, 184%, 185%, 186%, 187%, 188%, 189%, 190%, 191%, 192%, 193%, 194%, 195%, 196%, 197%, 198%, 199%, 200%, 201%, 202%, 203%, 204%, 205%, 206%, 207%, 208%, 209%, 210%, 211%, 212%, 213%, 214%, 215%, 216%, 217%, 218%, 219%, 220%, 221%, 222%, 223%, 224%, 225%, 226%, 227%, 228%, 229%, 230%, 231%, 232%, 233%, 234%, 235%, 236%, 237%, 238%, 239%, 240%, 241%, 242%, 243%, 244%, 245%, 246%, 247%, 248%, 249%, 250%, 251%, 252%, 253%, 254%, 255%, 256%, 257%, 258%, 259%, 260%, 261%, 262%, 263%, 264%, 265%, 266%, 267%, 268%, 269%, 270%, 271%, 272%, 273%, 274%, 275%, 276%, 277%, 278%, 279%, 280%, 281%, 282%, 283%, 284%, 285%, 286%, 287%, 288%, 289%, 290%, 291%, 292%, 293%, 294%, 295%, 296%, 297%, 298%, 299%, 300%, 301%, 302%, 303%, 304%, 305%, 306%, 307%, 308%, 309%, 310%, 311%, 312%, 313%, 314%, 315%, 316%, 317%, 318%, 319%, 320%, 321%, 322%, 323%, 324%, 325%, 326%, 327%, 328%, 329%, 330%, 331%, 332%, 333%, 334%, 335%, 336%, 337%, 338%, 339%, 340%, 341%, 342%, 343%, 344%, 345%, 346%, 347%, 348%, 349%, 350%, 351%, 352%, 353%, 354%, 355%, 356%, 357%, 358%, 359%, 360%, 361%, 362%, 363%, 364%, 365%, 366%, 367%, 368%, 369%, 370%, 371%, 372%, 373%, 374%, 375%, 376%, 377%, 378%, 379%, 380%, 381%, 382%, 383%, 384%, 385%, 386%, 387%, 388%, 389%, 390%, 391%, 392%, 393%, 394%, 395%, 396%, 397%, 398%, 399%, 400%, 401%, 402%, 403%, 404%, 405%, 406%, 407%, 408%, 409%, 410%, 411%, 412%, 413%, 414%, 415%, 416%, 417%, 418%, 419%, 420%, 421%, 422%, 423%, 424%, 425%, 426%, 427%, 428%, 429%, 430%, 431%, 432%, 433%, 434%, 435%, 436%, 437%, 438%, 439%, 440%, 441%, 442%, 443%, 444%, 445%, 446%, 447%, 448%, 449%, 450%, 451%, 452%, 453%, 454%, 455%, 456%, 457%, 458%, 459%, 460%, 461%, 462%, 463%, 464%, 465%, 466%, 467%, 468%, 469%, 470%, 471%, 472%, 473%, 474%, 475%, 476%, 477%, 478%, 479%, 480%, 481%, 482%, 483%, 484%, 485%, 486%, 487%, 488%, 489%, 490%, 491%, 492%, 493%, 494%, 495%, 496%, 497%, 498%, 499%, 500%, 501%, 502%, 503%, 504%, 505%, 506%, 507%, 508%, 509%, 510%, 511%, 512%, 513%, 514%, 515%, 516%, 517%, 518%, 519%, 520%, 521%, 522%, 523%, 524%, 525%, 526%, 527%, 528%, 529%, 530%, 531%, 532%, 533%, 534%, 535%, 536%, 537%, 538%, 539%, 540%, 541%, 542%, 543%, 544%, 545%, 546%, 547%, 548%, 549%, 550%, 551%, 552%, 553%, 554%, 555%, 556%, 557%, 558%, 559%, 560%, 561%, 562%, 563%, 564%, 565%, 566%, 567%, 568%, 569%, 570%, 571%, 572%, 573%, 574%, 575%, 576%, 577%, 578%, 579%, 580%, 581%, 582%, 583%, 584%, 585%, 586%, 587%, 588%, 589%, 590%, 591%, 592%, 593%, 594%, 595%, 596%, 597%, 598%, 599%, 600%, 601%, 602%, 603%, 604%, 605%, 606%, 607%, 608%, 609%, 610%, 611%, 612%, 613%, 614%, 615%, 616%, 617%, 618%, 619%, 620%, 621%, 622%, 623%, 624%, 625%, 626%, 627%, 628%, 629%, 630%, 631%, 632%, 633%, 634%, 635%, 636%, 637%, 638%, 639%, 640%, 641%, 642%, 643%, 644%, 645%, 646%, 647%, 648%, 649%, 650%, 651%, 652%, 653%, 654%, 655%, 656%, 657%, 658%, 659%, 660%, 661%, 662%, 663%, 664%, 665%, 666%, 667%, 668%, 669%, 670%, 671%, 672%, 673%, 674%, 675%, 676%, 677%, 678%, 679%, 680%, 681%, 682%, 683%, 684%, 685%, 686%, 687%, 688%, 689%, 690%, 691%, 692%, 693%, 694%, 695%, 696%, 697%, 698%, 699%, 700%, 701%, 702%, 703%, 704%, 705%, 706%, 707%, 708%, 709%, 710%, 711%, 712%, 713%, 714%, 715%, 716%, 717%, 718%, 719%, 720%, 721%, 722%, 723%, 724%, 725%, 726%, 727%, 728%, 729%, 730%, 731%, 732%, 733%, 734%, 735%, 736%, 737%, 738%, 739%, 740%, 741%, 742%, 743%, 744%, 745%, 746%, 747%, 748%, 749%, 750%, 751%, 752%, 753%, 754%, 755%, 756%, 757%, 758%, 759%, 760%, 761%, 762%, 763%, 764%, 765%, 766%, 767%, 768%, 769%, 770%, 771%, 772%, 773%, 774%, 775%, 776%, 777%, 778%, 779%, 780%, 781%, 782%, 783%, 784%, 785%, 786%, 787%, 788%, 789%, 790%, 791%, 792%, 793%, 794%, 795%, 796%, 797%, 798%, 799%, 800%, 801%, 802%, 803%, 804%, 805%, 806%, 807%, 808%, 809%, 810%, 811%, 812%, 813%, 814%, 815%, 816%, 817%, 818%, 819%, 820%, 821%, 822%, 823%, 824%, 825%, 826%, 827%, 828%, 829%, 830%, 831%, 832%, 833%, 834%, 835%, 836%, 837%, 838%, 839%, 840%, 841%, 842%, 843%, 844%, 845%, 846%, 847%, 848%, 849%, 850%, 851%, 852%, 853%, 854%, 855%, 856%, 857%, 858%, 859%, 860%, 861%, 862%, 863%, 864%, 865%, 866%, 867%, 868%, 869%, 870%, 871%, 872%, 873%, 874%, 875%, 876%, 877%, 878%, 879%, 880%, 881%, 882%, 883%, 884%, 885%, 886%, 887%, 888%, 889%, 890%, 891%, 892%, 893%, 894%, 895%, 896%, 897%, 898%, 899%, 900%, 901%, 902%, 903%, 904%, 905%, 906%, 907%, 908%, 909%, 910%, 911%, 912%, 913%, 914%, 915%, 916%, 917%, 918%, 919%, 920%, 921%, 922%, 923%, 924%, 925%, 926%, 927%, 928%, 929%, 930%, 931%, 932%, 933%, 934%, 935%, 936%, 937%, 938%, 939%, 940%, 941%, 942%, 943%, 944%, 945%, 946%, 947%, 948%, 949%, 950%, 951%, 952%, 953%, 954%, 955%, 956%, 957%, 958%, 959%, 960%, 961%, 962%, 963%, 964%, 965%, 966%, 967%, 968%, 969%, 970%, 971%, 972%, 973%, 974%, 975%, 976%, 977%, 978%, 979%, 980%, 981%, 982%, 983%, 984%, 985%, 986%, 987%, 988%, 989%, 990%, 991%, 992%, 993%, 994%, 995%, 996%, 997%, 998%, 999%, 1000% or more as compared to cells not in areas or having concentrated ATP or not having FcRN.

### Loading Milk Exosomes

In certain example embodiments, the method further includes loading the exosomes of the formulation resulting from the method described herein, with one or more cargos. The exosomes can be loaded by any suitable method. Exemplary methods of loading the milk exosomes, such as those prepared by a method described herein, are any of those set forth in International Patent Application Publication WO2020/028439, particularly at pages 83-87.

### Exemplary Exogenous Cargos

The milk exosomes can be loaded with any suitable or desired cargo(s). In some embodiments, the exogenous cargo(s) are therapeutic compounds or molecules. Exemplary cargos include, but are not limited to, nucleic acids (e.g., DNA, RNA), amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, radiation sensitizers, chemotherapeutics, imaging agents, immunogens, anti-cancer drugs, nutrients, polynucleotide modification system, or any combination thereof, and/or the like. In some embodiments, the exogenous cargo is a biologic molecule. In some embodiments, the exogenous cargo contains ester linked groups aiding uptake into the exosome.

In certain example embodiments, the exogenous cargo is any of those cargos set forth in WO 2020/028439, an ACT1 or ACT11 peptide, an ACT minus peptide (see e.g., ACT minus peptides set forth in WO 2022/076932, including but not limited to ACT minus I peptide), Gap19, JM2 (see e.g., U.S. Pat. No. 9,345,744 B2), a selectide (see e.g., WO 2022/076932), or any combination thereof. In certain example embodiments, the cargo is a peptide, including but not limited to an ACT-11 peptide. In certain example embodiments, the cargo is a peptide, including but not limited to an ACT-11-minus I peptide. Other peptide cargos include those set forth in International Patent Application Publication WO2020/028439, particularly at pages 67-82 and 85 and 106-111. In some embodiments, the cargo compound is esterified, such as described in International Patent Application Publication WO2020/028439 at page 81-86. In some embodiments, the cargo compound has multiple esterifications, such as described in International Patent Application Publication WO2020/028439 at page 81-86.

Exemplary nutrients include, without limitation, minerals (e.g., potassium, sodium, chloride, magnesium, manganese, cobalt, molybdenum, calcium, copper, zinc, iodine, iron, chromium, fluoride, selenium, etc.), vitamins (vitamin A, E, D, C, K, etc.), creatine, ATP, ADP, sugars (e.g., glucose, fructose, mannose, galactose, lactose, etc.), and fats and fatty acids.

Exemplary hormones include, but are not limited to, amino-acid derived hormones (e.g., melatonin and thyroxine), small peptide hormones and protein hormones (e.g., thyrotropin-releasing hormone, vasopressin, insulin, growth hormone, luteinizing hormone, follicle-stimulating hormone, and thyroid-stimulating hormone, CCK, obestatin, leptin, ghrelin, etc.), eicosanoids (e.g., arachidonic acid, lipoxins, and prostaglandins), purines (e.g., ATP), enzymes (e.g., creatine) and steroid hormones (e.g. estradiol, testosterone, tetrahydro testosterone, cortisol).

Exemplary immunomodulators include, but are not limited to, prednisone, azathioprine, 6-MP, cyclosporine, tacrolimus, methotrexate, interleukins (e.g., IL-2, IL-7, and IL-12), cytokines (e.g. interferons (e.g. IFN-α, IFN-β, IFN-ε, IFN-K, IFN-ω, and IFN-γ), granulocyte colony-stimulating factor, and imiquimod), chemokines (e.g. CCL3, CCL26 and CXCL7), cytosine phosphate-guanosine, oligodeoxynucleotides, glucans, antibodies, and aptamers).

Exemplary antipyretics include, but are not limited to, non-steroidal anti-inflammatories (e.g., ibuprofen, naproxen, ketoprofen, and nimesulide), aspirin and related salicylates (e.g., choline salicylate, magnesium salicylate, and sodium salicylate), paracetamol/acetaminophen, metamizole, nabumetone, phenazone, and quinine.

Exemplary anxiolytics include, but are not limited to, benzodiazepines (e.g., alprazolam, bromazepam, chlordiazepoxide, clonazepam, clorazepate, diazepam, flurazepam, lorazepam, oxazepam, temazepam, triazolam, and tofisopam), serotonergic antidepressants (e.g., selective serotonin reuptake inhibitors, tricyclic antidepressants, and monoamine oxidase inhibitors), temgicoluril, fabomotizole, selank, bromantane, emoxypine, azapirones, barbiturates, hydroxyzine, pregabalin, isovaleric acid, and beta blockers.

Exemplary antipsychotics include, but are not limited to, benperidol, bromperidol, droperidol, haloperidol, moperone, pipamperone, timiperone, fluspirilene, penfluridol, pimozide, acepromazine, chlorpromazine, cyamemazine, dixyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, tiotixene, zuclopenthixol, clotiapine, loxapine, prothipendyl, carpipramine, clocapramine, molindone, mosapramine, sulpiride, veralipride, amisulpride, amoxapine, aripiprazole, asenapine, clozapine, blonanserin, iloperidone, lurasidone, melperone, nemonapride, olanzapine, paliperidone, perospirone, quetiapine, remoxipride, risperidone, sertindole, trimipramine, ziprasidone, zotepine, alstonie, bifeprunox, bitopertin, brexpiprazole, cannabidiol, cariprazine, pimavanserin, pomaglumetad methionil, vabicaserin, xanomeline, and zicronapine.

Exemplary analgesics include, but are not limited to, paracetamol/acetaminophen, nonsteroidal anti-inflammantories (e.g. ibuprofen, naproxen, ketoprofen, and nimesulide), COX-2 inhibitors (e.g., rofecoxib, celecoxib, and etoricoxib), opioids (e.g. morphine, codeine, oxycodone, hydrocodone, dihydromorphine, pethidine, buprenorphine), tramadol, norepinephrine, flupirtine, nefopam, orphenadrine, pregabalin, gabapentin, cyclobenzaprine, scopolamine, methadone, ketobemidone, piritramide, and aspirin and related salicylates (e.g. choline salicylate, magnesium salicylate, and sodium salicylate).

Exemplary antispasmodics include, but are not limited to, mebeverine, papaverine, cyclobenzaprine, carisoprodol, orphenadrine, tizanidine, metaxalone, methocarbamol, chlorzoxazone, baclofen, dantrolene, baclofen, tizanidine, and dantrolene. Suitable anti-inflammatories include, but are not limited to, prednisone, non-steroidal anti-inflammantories (e.g., ibuprofen, naproxen, ketoprofen, and nimesulide), COX-2 inhibitors (e.g., rofecoxib, celecoxib, and etoricoxib), and immune selective anti-inflammatory derivatives (e.g., submandibular gland peptide-T and its derivatives).

Exemplary anti-histamines include, but are not limited to, H1-receptor antagonists (e.g., acrivastine, azelastine, bilastine, brompheniramine, buclizine, bromodiphenhydramine, carbinoxamine, cetirizine, chlorpromazine, cyclizine, chlorpheniramine, clemastine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocetirizine, loratadine, meclizine, mirtazapine, olopatadine, orphenadrine, phenindamine, pheniramine, phenyltoloxamine, promethazine, pyrilamine, quetiapine, rupatadine, tripelennamine, and triprolidine), H2-receptor antagonists (e.g., cimetidine, famotidine, lafutidine, nizatidine, ranitidine, and roxatidine), tritoqualine, catechin, cromoglicate, nedocromil, and p2-adrenergic agonists.

Exemplary anti-infectives include, but are not limited to, amebicides (e.g., nitazoxanide, paromomycin, metronidazole, tinidazole, chloroquine, miltefosine, amphotericin b, and iodoquinol), aminoglycosides (e.g., paromomycin, tobramycin, gentamicin, amikacin, kanamycin, and neomycin), anthelmintics (e.g., pyrantel, mebendazole, ivermectin, praziquantel, albendazole, thiabendazole, oxamniquine), antifungals (e.g., azole antifungals (e.g., itraconazole, fluconazole, posaconazole, ketoconazole, clotrimazole, miconazole, and voriconazole), echinocandins (e.g., caspofungin, anidulafungin, and micafungin), griseofulvin, terbinafine, flucytosine, and polyenes (e.g., nystatin, and amphotericin b), antimalarial agents (e.g., pyrimethamine/sulfadoxine, artemether/lumefantrine, atovaquone/proquanil, quinine, hydroxychloroquine, mefloquine, chloroquine, doxycycline, pyrimethamine, and halofantrine), antituberculosis agents (e.g., aminosalicylates (e.g., amino salicylic acid), isoniazid/rifampin, isoniazid/pyrazinamide/rifampin, bedaquiline, isoniazid, ethambutol, rifampin, rifabutin, rifapentine, capreomycin, and cycloserine), antivirals (e.g., amantadine, rimantadine, abacavir/lamivudine, emtricitabine/tenofovir, cobicistat/elvitegravir/emtricitabine/tenofovir, efavirenz/emtricitabine/tenofovir, abacavir/lamivudine/zidovudine, lamivudine/zidovudine, emtricitabine/tenofovir, emtricitabine/lopinavir/ritonavir/tenofovir, interferon alfa-2v/ribavirin, peginterferon alfa-2b, maraviroc, raltegravir, dolutegravir, enfuvirtide, foscarnet, fomivirsen, oseltamivir, zanamivir, nevirapine, efavirenz, etravirine, rilpivirine, delavirdine, nevirapine, entecavir, lamivudine, adefovir, sofosbuvir, didanosine, tenofovir, abacavir, zidovudine, stavudine, emtricitabine, zalcitabine, telbivudine, simeprevir, boceprevir, telaprevir, lopinavir/ritonavir, fosamprenavir, darunavir, ritonavir, tipranavir, atazanavir, nelfinavir, amprenavir, indinavir, saquinavir, ribavirin, valacyclovir, acyclovir, famciclovir, ganciclovir, and valganciclovir), carbapenems (e.g., doripenem, meropenem, ertapenem, and cilastatin/imipenem), cephalosporins (e.g., cefadroxil, cephradine, cefazolin, cephalexin, cefepime, ceflaroline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, cefizoxime, and ceftazidime), glycopeptide antibiotics (e.g., vancomycin, dalbavancin, oritavancin, and telavancin), glycylcyclines (e.g. tigecycline), leprostatics (e.g. clofazimine and thalidomide), lincomycin and derivatives thereof (e.g. clindamycin and lincomycin), macrolides and derivatives thereof (e.g. telithromycin, fidaxomicin, erythromycin, azithromycin, clarithromycin, dirithromycin, and troleandomycin), linezolid, sulfamethoxazole/trimethoprim, rifaximin, chloramphenicol, Fosfomycin, metronidazole, aztreonam, bacitracin, penicillins (amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxacillin, dicloxacillin, and nafcillin), quinolones (e.g., lomefloxacin, norfloxacin, ofloxacin, moxifloxacin, ciprofloxacin, levofloxacin, Gemifloxacin, moxifloxacin, cinoxacin, nalidixic acid, enoxacin, grepafloxacin, gatifloxacin, trovafloxacin, and sparfloxacin), sulfonamides (e.g., sulfamethoxazole/trimethoprim, sulfasalazine, and sulfasoxazole), tetracyclines (e.g., doxycycline, demeclocycline, minocycline, doxycycline/salicylic acid, doxycycline/omega-3 polyunsaturated fatty acids, and tetracycline), and urinary anti-infectives (e.g., nitrofurantoin, methenamine, fosfomycin, cinoxacin, nalidixic acid, trimethoprim, and methylene blue).

Exemplary chemotherapeutics include, but are not limited to, paclitaxel, herceptin, brentuximab vedotin, doxorubicin, 5-FU (fluorouracil), everolimus, pemetrexed, melphalan, pamidronate, anastrozole, exemestane, nelarabine, ofatumumab, bevacizumab, belinostat, tositumomab, carmustine, bleomycin, bosutinib, busulfan, alemtuzumab, irinotecan, vandetanib, bicalutamide, lomustine, daunorubicin, clofarabine, cabozantinib, dactinomycin, ramucirumab, cytarabine, Cytoxan, cyclophosphamide, decitabine, dexamethasone, docetaxel, hydroxyurea, dacarbazine, leuprolide, epirubicin, oxaliplatin, asparaginase, estramustine, cetuximab, vismodegib, asparaginase Erwinia chrysanthemin, amifostine, etoposide, flutamide, toremifene, fulvestrant, letrozole, degarelix, pralatrexate, methotrexate, floxuridine, obinutuzumab, gemcitabine, afatinib, imatinib mesylate, carmustine, eribulin, trastuzumab, altretamine, topotecan, ponatinib, idarubicin, ifosfamide, ibrutinib, axitinib, interferon alfa-2a, gefitinib, romidepsin, ixabepilone, ruxolitinib, cabazitaxel, ado-trastuzumab emtansine, carfilzomib, chlorambucil, sargramostim, cladribine, mitotane, vincristine, procarbazine, megestrol, trametinib, mesna, strontium-89 chloride, mechlorethamine, mitomycin, busulfan, gemtuzumab ozogamicin, vinorelbine, filgrastim, pegfilgrastim, sorafenib, nilutamide, pentostatin, tamoxifen, mitoxantrone, pegaspargase, denileukin diftitox, alitretinoin, carboplatin, pertuzumab, cisplatin, pomalidomide, prednisone, aldesleukin, mercaptopurine, zoledronic acid, lenalidomide, rituximab, octreotide, dasatinib, regorafenib, histrelin, sunitinib, siltuximab, omacetaxine, thioguanine (tioguanine), dabrafenib, erlotinib, bexarotene, temozolomide, thiotepa, thalidomide, BCG, temsirolimus, bendamustine hydrochloride, triptorelin, arsenic trioxide, lapatinib, valrubicin, panitumumab, vinblastine, bortezomib, tretinoin, azacitidine, pazopanib, teniposide, leucovorin, crizotinib, capecitabine, enzalutamide, ipilimumab, goserelin, vorinostat, idelalisib, ceritinib, abiraterone, epothilone, tafluposide, azathioprine, doxifluridine, vindesine, and all-trans retinoic acid.

Suitable radiation sensitizers include, but are not limited to, 5-fluorouracil, platinum analogs (e.g., cisplatin, carboplatin, and oxaliplatin), gemcitabine, DNA topoisomerase I-targeting drugs (e.g., camptothecin derivatives (e.g., topotecan and irinotecan)), epidermal growth factor receptor blockade family agents (e.g., cetuximab, gefitinib), farnesyltransferase inhibitors (e.g., L-778-123), COX-2 inhibitors (e.g., rofecoxib, celecoxib, and etoricoxib), bFGF and VEGF targeting agents (e.g., bevazucimab and thalidomide), NBTXR3, Nimoral, trans sodium crocetinate, NVX-108, and combinations thereof. See also e.g., Kvols, L. K., J Nucl Med 2005; 46:187S-190S.

Other exemplary cargos include anticoagulants (e.g., heparin and heparin variants).

Exemplary immunogens, carried as cargo or attached to the external surface of the isolated exosomes could include Keyhole Limpet Hemocyanin (KLH), Concholepas Concholepas Hemocyanin (CCH), (also Blue Carrier Immunogenic Protein), Bovine Serum Albumin (BSA), Ovalbumin (OVA), and antigens used to generate immune responses to pathogens causing disease including that causing diphtheria, tetanus, pertussis, measles, mumps, rubella, hepatitis A, hepatitis B, meningococcal disease (e.g., meningitis), human papillomavirus varicella, rabies, flu, rotoviral, HIV, malarial and coronaviral disease.

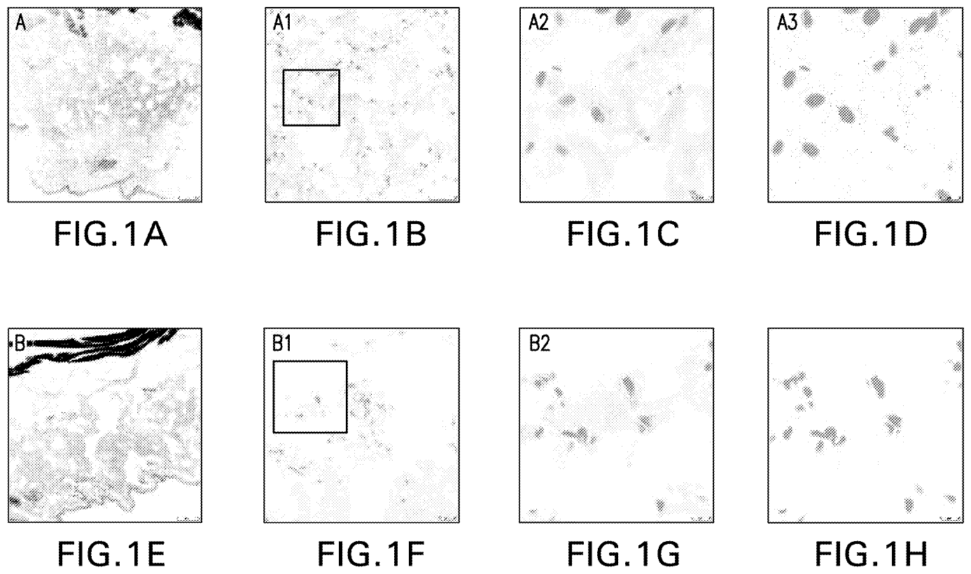

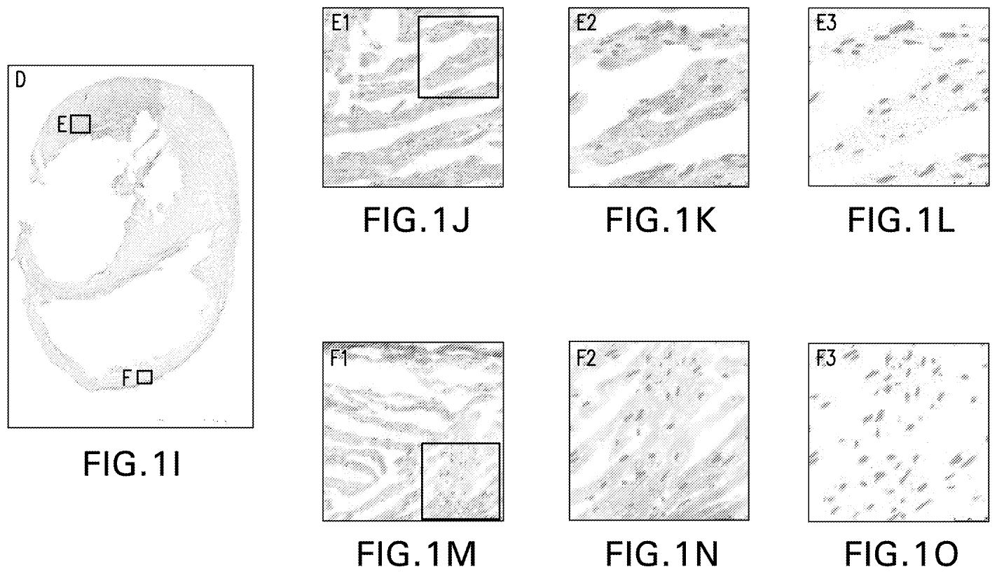

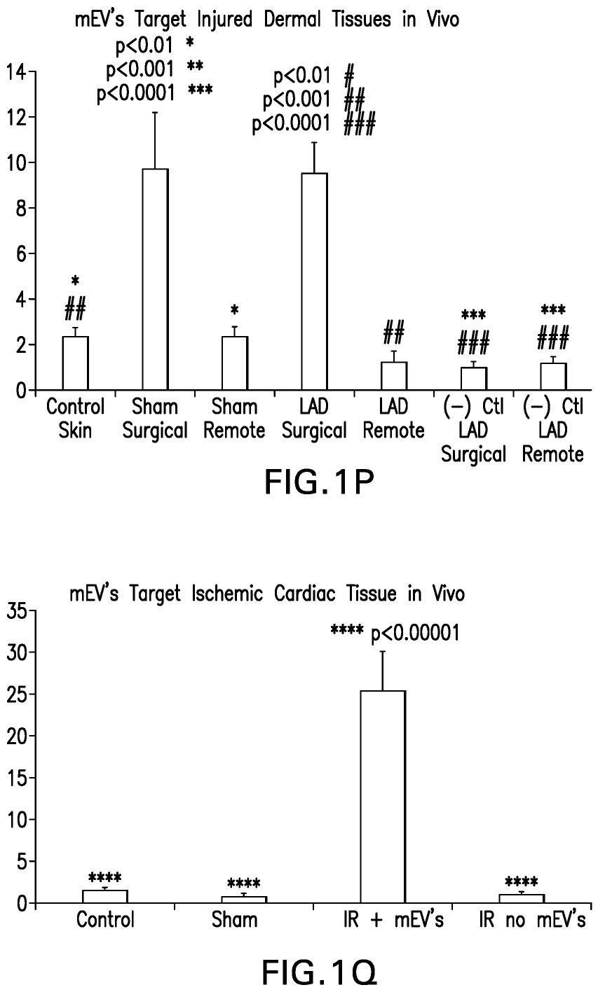

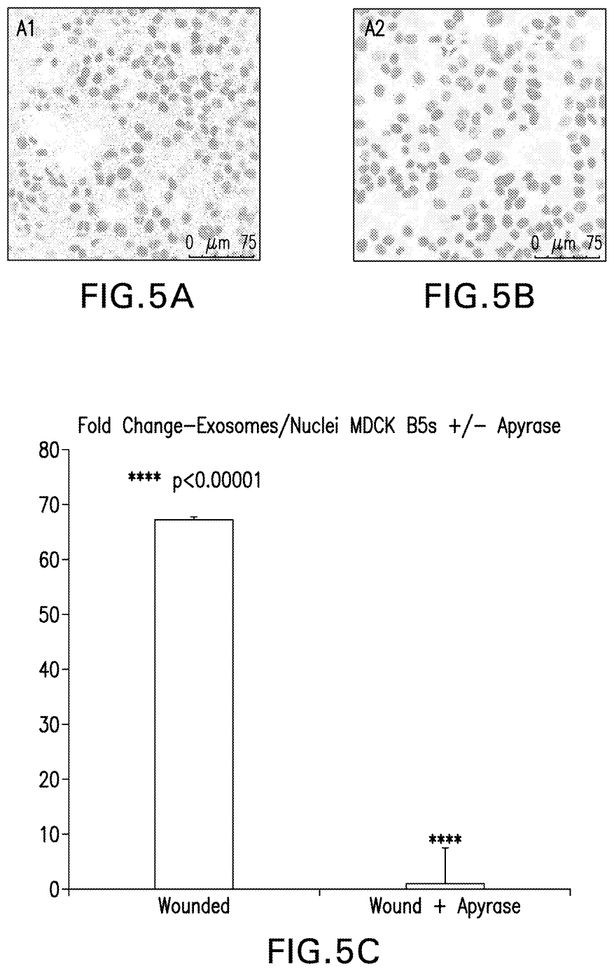

Exemplary polynucleotide modification systems, include without limitation, CRISPR-Cas systems, OMEGA systems, PRIME editing systems, base editors, meganucleases, zinc-finger nucleases, recombinases, TALE nucleases, CAST systems, Non-LTR retrotransposon systems, transposons, RNAi, antisense nucleic acids, etc. See e.g., Crooke S T, Liang X H, Baker B F, Crooke R M. Antisense technology: A review. J Biol Chem. 2021; 296:100416. doi:10.1016/j.jbc.2021.100416; Shmakov et al. (2015) “Discovery and Functional Characterization of Diverse Class 2 CRISPR-Cas Systems”, Molecular Cell, DOI: dx.doi.org/10.1016/j.molcel.2015.10.008; Peters et al., PNAS 114 (35) (2017); DOI: 10.1073/pnas.1709035114; Makarova et al. 2018. The CRISPR Journal, v. 1, n5, FIG. 5; Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160; Moscou et al., Science 326:1501 (2009); Boch et al., Science 326:1509-1512 (2009); and Zhang et al., Nature Biotechnology 29:149-153 (2011); Rees and Liu. 2018. Nat. Rev. Gent. 19(12):770-788; Komor et al. 2016. Nature. 533:420-424; Nishida et al. 2016. Science. 353; Gaudeli et al. 2017. Nature. 551:464-471; Cox et al. 2017. Science 358: 1019-1027; Levy et al. Nature Biomedical Engineering doi.org/10.1038/s41441-019-0505-5 (2019); Gorsuch et al. (2022). Targeting the hepatitis B cccdna with a sequence-specific arcus nuclease to eliminate hepatitis B virus in vivo. Molecular Therapy, 30(9), 2909-2922. doi.org/10.1016/j.ymthe.2022.05.013; Anzalone et al. 2019. Nature. 576: 149-157, particularly at FIGS. 1b, 1c, related discussion, and Supplementary discussion; Anzalone A V, Gao X D, Podracky C J, et al. Programmable deletion, replacement, integration and inversion of large DNA sequences with twin prime editing. Nat Biotechnol. 2022; 40(5):731-740; Yarnall et al., Nat Biotechnol (2022). doi.org/10.1038/s41587-022-01527-4; Groth, A. C. and Calos, M. P. (2004) J. Mol. Biol. 335, 667-678; Lei, et al., FEBS Lett. 2018 April; 592(8):1389-1399; Singh, et al., Attachment Site Selection and Identity in Bxbl Serine Integrase-Mediated Site-Specific Recormbination, PLoS Genet. 2013 May; 9(5):e1003490; and Gupta, et al., Nucleic Acids Res. 2007 May; 35(10): 3407-3419; Tou et al. bioRxiv 2022.01.07.475005, doi.org/10.1101/2022.01.07.475005; Klompe et al. Nature, doi:10.1038/s41586-019-1323; Strecker et al. Science. 10/1126/science. aax9181 (2019); Christensen S M et al., RNA from the 5′ end of the R2 retrotransposon controls R2 protein binding to and cleavage of its DNA target site, Proc Natl Acad Sci USA. 2006 Nov. 21; 103(47):17602-7; Eickbush T H et al, Integration, Regulation, and Long-Term Stability of R2 Retrotransposons, Microbiol Spectr. 2015 April; 3(2):MDNA3-0011-2014. doi: 10.1128/microbiolspec.MDNA3-0011-2014; Han J S, Non-long terminal repeat (non-LTR) retrotransposons: mechanisms, recent developments, and unanswered questions, Mob DNA. 2010 May 12; 1(1):15. doi: 10.1186/1759-8753-1-15; Malik H S et al., The age and evolution of non-LTR retrotransposable elements, Mol Biol Evol. 1999 June; 16(6):793-805; U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, 6,479,626, 8,163,514, 8,133,697, 8,021,867, 8,119,361, 8,119,381, 8,124,369, 8,129,134, 10,851,358; U.S. Patent Application Publication No. 2020/0239544, 2018/0346934; International Patent Application Publications WO 2018/213708, WO 2018/213726, WO 2014/093622, WO 2019/005884, WO 2019/005886, WO 2019/071048, WO 2016/106236, WO 2020/206231; WO 2021/138469, WO 2020/131862, WO 2021/257997, WO 2021/087394, WO 2022/147321, WO 2022/07682, WO 2022/150651, WO 2021/102042, WO 2022/173830.

In some embodiments, the exogenous cargo is present in the milk exosome at any non-zero amount ranging from 0 to/or 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges.

### Exemplary Methods of Isolating Milk Exosomes

The milk exosomes can be isolated from a mammalian milk using any appropriate technique. In some embodiments, the mammalian milk is bovine, ovine, or camelid milk. In some embodiments, the milk exosome or population thereof is isolated according to a method described in Marsh et al., Nanotheranostics. 2021 Jul. 5; 5(4):488-498. doi: 10.7150/ntno.62213. eCollection 2021 and WO 2022/182782, which are incorporated by reference as if expressed in their entireties herein). In some embodiments, the milk exosome(s) is isolated using a method including the steps of (a) centrifuging a mammalian milk under conditions suitable to separate fats from one or more other components of the mammalian milk; (b) removing the separated fats from the mammalian milk; (c) after step (b) centrifuging the remaining mammalian milk one or more times and skimming any noticeable separated fats after each centrifuging in step (c); (d) filtering the remaining biological fluid after step (c); (e) optionally performing one or more ultracentrifugation steps after (d); (f) chelating divalent cations with about 10 mM to about 100 mM EDTA at about 30-42 degrees Celsius after (d) or optionally (e) and optionally for about 15-120 minutes; and (g) after (f), optionally performing tangential flow filtration to obtain a retentate, wherein the retentate is optionally ultracentrifuged via one or more ultracentrifugation steps or stored at −80 degrees C., and separating out fractions of the retentate, optionally via column separation, after the retentate is optionally ultracentrifuged or stored at −80 degrees C., wherein the method comprises step (e) or step (g) but not both.

### Milk Exosome Formulations and Kits

Also described herein are pharmaceutical formulations that can contain an amount, effective amount, and/or least effective amount, and/or therapeutically effective amount of one or more milk exosomes, such as exogenous cargo loaded milk exosomes, described in greater detail elsewhere herein and a pharmaceutically acceptable carrier or excipient. Described in certain example embodiments herein are methods that include administering a formulation as described in any one of the previous paragraphs and/or elsewhere herein, such as the Working Examples below, to a subject. In some embodiments, the formulation administered to the subject includes milk exosomes, such as any of those described elsewhere herein and/or prepared by a method described elsewhere herein. In some embodiments, the milk exosomes are exogenous cargo loaded milk exosomes.

In some embodiments the subject to which the milk exosomes or formulation thereof is administered to a subject has a disease, disorder, condition, or an injury (e.g., a mechanical or non-mechanical injury). Exemplary diseases disorders, conditions, and injuries include, but are not limited to, a cancer, a viral infection, a bacterial infection, a parasite infection, a mechanical injury (e.g., external and internal wounds and tissue injuries, cuts, scrapes, burns, surgical wounds, wounds secondary to infection, disease, or condition, etc.), cancer, non-mechanical injury (e.g., chemical injury, electrical injury, radiation injury, ischemic and/or hypoxic injuries (e.g. myocardial infarction, ischemic wounds and/or stroke), multiple sclerosis, psoriasis, scleroderma, acne, eczema, or a disease of the skin and/or connective tissues, cardiac diseases or disorders, neurodegenerative diseases or disorders, neurological disorders, atherosclerosis, pathologies involving epithelial permeabilization and/or neovascularization (e.g., angiogenesis or vasculogenesis), respiratory distress syndrome (RDS), reperfusion injuries, dermal vascular blemish or malformation, macular degeneration, neovascularization of choriocapillaries through Bruch's membrane, diabetic retinopathy, inflammatory injury, inflammatory and inflammation-related diseases and disorders), and radiation dermatitis.

Wounds can be chronic wounds or wounds that appear to not completely heal. Wounds that have not healed within three months, for example, are said to be chronic. Chronic wounds include, diabetic foot ulcers, ischemic, venous ulcers, venous leg ulcers, venous stasis, arterial, pressure, vasculitic, infectious, decubitis, burn, trauma-induced, gangrenous and mixed ulcers. Chronic wounds include wounds that are characterized by and/or chronic inflammation, deficient and overprofuse granulation tissue differentiation and failure of re-epithelialization and wound closure and longer repair times. Chronic wounds can include ocular ulcers, including corneal ulcers. Use of the disclosed invention in wound healing and tissue regeneration can include in humans and agricultural, sports and pet animals.

Tissue injuries can result from, for example, a cut, scrape, compression wound, stretch injury, laceration wound, crush wound, bite wound, graze, bullet wound, explosion injury, body piercing, stab wound, surgical wound, surgical intervention, medical intervention, host rejection following cell, tissue or organ grafting, pharmaceutical effect, pharmaceutical side-effect, bed sore, radiation injury, radiation illness, inflammation, cosmetic skin wound, internal organ injury, disease process (e.g., asthma, cancer), infection, infectious agent, developmental process, maturational process (e.g., acne), genetic abnormality, developmental abnormality, environmental toxin, allergen, scalp injury, facial injury, jaw injury, sex organ injury, joint injury, excretory organ injury, foot injury, finger injury, toe injury, bone injury, eye injury, corneal injury, muscle injury, adipose tissue injury, lung injury, airway injury, hernia, anus injury, piles, ear injury, skin injury, abdominal injury, retinal injury, eye injury, corneal injury, arm injury, leg injury, athletic injury, back injury, birth injury, premature birth injury, toxic bite, sting, injury to barrier function, injury to endothelial barrier function, injury to epithelial barrier function, tendon injury, ligament injury, heart injury, heart valve injury, vascular system injury, cartilage injury, lymphatic system injury, craniocerebral trauma, dislocation, esophageal perforation, fistula, nail injury, foreign body, fracture, frostbite, hand injury, heat stress disorder, laceration, neck injury, self-mutilation, shock, traumatic soft tissue injury, spinal cord injury, spinal injury, sprain, strain, tendon injury, ligament injury, cartilage injury, thoracic injury, tooth injury, trauma, nervous system injury, burn, burn wound, wind burn, sun burn, chemical burn, aging, aneurism, stroke, surgical radiation injury, digestive tract injury, infarct, or ischemic injury.

Cardiac diseases and disorders can include, but are not limited to, myocardial infarction, cardio myopathies (e.g., hypertrophic cardiomyopathy), arrhythmias, congestive heart failure. The regenerative effects of the provided composition may result in beneficial changes in membrane excitability and ion transients of the heart. There are many different types of arrhythmia that can lead to abnormal function in the human heart. Arrhythmias include, but are not limited to bradycardias, tachycardias, alternans, automaticity defects, reentrant arrhythmias, fibrillation, AV nodal arrhythmias, atrial arrhythmias and triggered beats, Long QT syndrome, Short QT syndrome, Brugada syndrome, premature atrial Contractions, wandering Atrial pacemaker, Multifocal atrial tachycardia, Atrial flutter, Atrial fibrillation, Supraventricular tachycardia, AV nodal reentrant tachycardia is the most common cause of Paroxysmal Supraventricular Tachycardia, Junctional rhythm, Junctional tachycardia, Premature junctional complex, Wolff-Parkinson-White syndrome, Lown-Ganong-Levine syndrome, Premature Ventricular Contractions (PVC) sometimes called Ventricular Extra Beats, alternans and discordant alternans, Accelerated idioventricular rhythm, Monomorphic Ventricular tachycardia, Polymorphic ventricular tachycardia, Ventricular fibrillation, First degree heart block, which manifests as PR prolongation, Second degree heart block, Type 1 Second degree heart block, Type 2 Second degree heart block, Third degree heart block, and several accessory pathway disorders (e.g., Wolff-Parkinson-White syndrome (WPW)).

Neurodegenerative and neurological disorders include, but are not limited to dementia, Alzheimer's disease, Parkinson's disease and related PD-diseases, amyotrophic lateral sclerosis (ALS), motor neuron disease, schizophrenia, spinocerebellar ataxia, prion disease, Spinal muscular atrophy (SMA), multiple sclerosis, epilepsy and other seizure disorders, and Huntington's disease.

Inflammatory diseases and inflammatory-related diseases and disorders can be asthma, eczema, sinusitis, atherosclerosis, arthritis (including but not limited to rheumatoid arthritis), inflammatory bowel disease, cutaneous and systemic mastocytosis, psoriasis, and multiple sclerosis. As used herein, the term “inflammatory disorder” can include diseases or disorders which are caused, at least in part, or exacerbated, by inflammation, which is generally characterized by increased blood flow, edema, activation of immune cells (e.g., proliferation, cytokine production, or enhanced phagocytosis), heat, redness, swelling, pain and/or loss of function in the affected tissue or organ. The cause of inflammation can be due to physical damage, chemical substances, micro-organisms, tissue necrosis, cancer, or other agents or conditions.

Inflammatory disorders include acute inflammatory disorders, chronic inflammatory disorders, and recurrent inflammatory disorders. Acute inflammatory disorders are generally of relatively short duration, and last for from about a few minutes to about one to two days, although they can last several weeks. Characteristics of acute inflammatory disorders include increased blood flow, exudation of fluid and plasma proteins (edema) and emigration of leukocytes, such as neutrophils. Chronic inflammatory disorders, generally, are of longer duration, e.g., weeks to months to years or longer, and are associated histologically with the presence of lymphocytes and macrophages and with proliferation of blood vessels and connective tissue. Recurrent inflammatory disorders include disorders which recur after a period of time or which have periodic episodes. Some inflammatory disorders fall within one or more categories. Exemplary inflammatory disorders include but are not limited to atherosclerosis; arthritis; inflammation-promoted cancers; asthma; autoimmune uveitis; adoptive immune response; dermatitis; multiple sclerosis; diabetic complications; osteoporosis; Alzheimer's disease; cerebral malaria; hemorrhagic fever; autoimmune disorders; and inflammatory bowel disease. In some embodiments, the inflammatory disorder is an autoimmune disorder that, in some embodiments, is selected from lupus, rheumatoid arthritis, and autoimmune encephalomyelitis.

In some embodiments, the inflammatory disorder is a brain-related inflammatory disorder. The term “brain-related inflammatory” disorder is used herein to refer to a subset of inflammatory disorders that are caused, at least in part, or originate or are exacerbated, by inflammation in the brain of a subject.

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo. As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. When present, the cargo can optionally be present in the pharmaceutical formulation as a pharmaceutically acceptable salt. In some embodiments, the pharmaceutical formulation can include, such as an active ingredient, one or more milk exosomes, such as cargo loaded milk exosomes, described in greater detail elsewhere herein.

In some embodiments, the cargo is present as a pharmaceutically acceptable salt of the active ingredient. As used herein, “pharmaceutically acceptable salt” refers to any acid or base addition salt whose counter-ions are non-toxic to the subject to which they are administered in pharmaceutical doses of the salts. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

The pharmaceutical formulations described herein can be administered to a subject in need thereof via any suitable method or route to a subject in need thereof. Suitable administration routes can include, but are not limited to auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the active ingredient(s) and/or cargos.

Where appropriate, one or more milk exosomes, such as cargo loaded milk exosomes, described in greater detail elsewhere herein can be provided to a subject in need thereof as an ingredient, such as an active ingredient or agent, in a pharmaceutical formulation. As such, also described are pharmaceutical formulations containing one or more milk exosomes, such as cargo loaded milk exosomes, described in greater detail elsewhere herein include a cargo that is in the form of a pharmaceutically acceptable salt. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

As used herein, “agent” refers to any substance, compound, molecule, and the like, which can be biologically active or otherwise can induce a biological and/or physiological effect on a subject to which it is administered to. As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed. In some embodiments the active agent is a milk exosome, or a cargo loaded milk exosome. In some embodiments, the active agent includes or is the cargo of a cargo loaded milk exosome.

In certain embodiments, the milk exosome is prepared by any method described elsewhere herein. In some embodiments, the cargo loaded milk exosome is as described and/or prepared by a method as described elsewhere herein.

In some embodiments, the milk exosomes or formulations thereof are included on a material to administer the milk exosomes or formulations thereof to a subject. Non-limiting examples of materials include those that used to treat wounds such as bandages, steri-strip, sutures, staples, or grafts (e.g., skin grafts). Other exemplary materials include medical devices or implants (or components thereof). Non-limiting examples of medical implants include: limb prostheses, breast implants, penile implants, testicular implants, artificial eyes, facial implants, artificial joints, heart valve prostheses, vascular prostheses, dental prostheses, facial prosthesis, tilted disc valve, caged ball valve, ear prosthesis, nose prosthesis, pacemakers, cochlear implants, stents, shunts, catheters, filters, meshes, fillers (e.g., fat and dermal filers), and skin substitutes (e.g., porcine heterograft/pigskin, BIOBRANE, cultured keratinocytes), and/or the like.

### Pharmaceutically Acceptable Carriers and Secondary Ingredients and Agents

The pharmaceutical formulation can include a pharmaceutically acceptable carrier. Suitable pharmaceutically acceptable carriers include, but are not limited to water, milk and milk products (e.g. casein, ice cream, custards, creamers, and/or the like), salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In some embodiments, the pharmaceutical formulation can also include an effective amount of secondary active agents, including but not limited to, biologic agents or molecules including, but not limited to, e.g. polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and any combination thereof.

In some embodiments, the secondary active agent is one or more immunoglobulins. In some embodiments, the one or more immunoglobulins are IgG, IgM, IgA, IgD, IgE, or any combination thereof. In some embodiments, the one or more immunoglobulins are included in the pharmaceutical formulation in a dosage from that releases the one or more immunoglobulins prior to delivery or release of the milk exosomes present in the pharmaceutical formulation.

In some embodiments, the secondary active agent is one or more agents that stimulating ATP release, secretion, and/or production, ATP, ADP, or any combination thereof. In some embodiments, the one or more agents that stimulating ATP release, secretion, and/or production, ATP, ADP, or any combination thereof is included in the pharmaceutical formulation in a dosage from that releases the one or more agents that stimulating ATP release, secretion, and/or production, ATP, ADP, or any combination thereof prior to delivery or release of the milk exosomes present in the pharmaceutical formulation.

In some embodiments, the secondary active agents include one or more immunoglobulins, one or more agents that stimulating ATP release, secretion, and/or production, ATP, ADP, or any combination thereof. In some embodiments, the one or more immunoglobulins, one or more agents that stimulating ATP release, secretion, and/or production, ATP and/or ADP release, secretion, and/or production, or any combination thereof are included in the pharmaceutical formulation in a dosage form that releases the one or more immunoglobulins, one or more agents that stimulating ATP release, secretion, and/or production, ATP and/or ADP release, secretion, and/or production or any combination thereof, prior to delivery or release of the milk exosomes present in the pharmaceutical formulation.

In some embodiments, the combination therapy results in increased uptake of the milk exosomes by a cell or cells. In some embodiments, the milk exosomes have increased uptake in cells that have released and concentrated ATP or are in environments with concentrated secreted ATP as compared to cells that do not have concentrated levels of released ATP or are in environments without concentrated ATP stimulated to be secreted by cells due to a prompt due such as injury or the presence of cancerous cells. In some embodiments, the milk exosomes have increased uptake in cells having FcRN on their surface. gastrointestinal cell, a heart cell, a liver cell, a brain cell, a nerve cell, a cancer cell, or any combination thereof. In some embodiments, the cell is a polar cell, meaning that the cells have a different apical and basal membrane surface. In some embodiments, the cell is an enterocyte. In some embodiments, the cells is a blood-brain barrier cell. In some embodiments, uptake by a cell is increased by the secondary ATP, ADP, immunoglobulin, and/or agent effective to stimulate ATP release, secretion, and/or production by 0.01%, to/or 0.02%, 0.03%, 0.04%, 0.05%, 0.06%, 0.07%, 0.08%, 0.09%, 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.31%, 0.32%, 0.33%, 0.34%, 0.35%, 0.36%, 0.37%, 0.38%, 0.39%, 0.4%, 0.41%, 0.42%, 0.43%, 0.44%, 0.45%, 0.46%, 0.47%, 0.48%, 0.49%, 0.5%, 0.51%, 0.52%, 0.53%, 0.54%, 0.55%, 0.56%, 0.57%, 0.58%, 0.59%, 0.6%, 0.61%, 0.62%, 0.63%, 0.64%, 0.65%, 0.66%, 0.67%, 0.68%, 0.69%, 0.7%, 0.71%, 0.72%, 0.73%, 0.74%, 0.75%, 0.76%, 0.77%, 0.78%, 0.79%, 0.8%, 0.81%, 0.82%, 0.83%, 0.84%, 0.85%, 0.86%, 0.87%, 0.88%, 0.89%, 0.9%, 0.91%, 0.92%, 0.93%, 0.94%, 0.95%, 0.96%, 0.97%, 0.98%, 0.99%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 101%, 102%, 103%, 104%, 105%, 106%, 107%, 108%, 109%, 110%, 111%, 112%, 113%, 114%, 115%, 116%, 117%, 118%, 119%, 120%, 121%, 122%, 123%, 124%, 125%, 126%, 127%, 128%, 129%, 130%, 131%, 132%, 133%, 134%, 135%, 136%, 137%, 138%, 139%, 140%, 141%, 142%, 143%, 144%, 145%, 146%, 147%, 148%, 149%, 150%, 151%, 152%, 153%, 154%, 155%, 156%, 157%, 158%, 159%, 160%, 161%, 162%, 163%, 164%, 165%, 166%, 167%, 168%, 169%, 170%, 171%, 172%, 173%, 174%, 175%, 176%, 177%, 178%, 179%, 180%, 181%, 182%, 183%, 184%, 185%, 186%, 187%, 188%, 189%, 190%, 191%, 192%, 193%, 194%, 195%, 196%, 197%, 198%, 199%, 200%, 201%, 202%, 203%, 204%, 205%, 206%, 207%, 208%, 209%, 210%, 211%, 212%, 213%, 214%, 215%, 216%, 217%, 218%, 219%, 220%, 221%, 222%, 223%, 224%, 225%, 226%, 227%, 228%, 229%, 230%, 231%, 232%, 233%, 234%, 235%, 236%, 237%, 238%, 239%, 240%, 241%, 242%, 243%, 244%, 245%, 246%, 247%, 248%, 249%, 250%, 251%, 252%, 253%, 254%, 255%, 256%, 257%, 258%, 259%, 260%, 261%, 262%, 263%, 264%, 265%, 266%, 267%, 268%, 269%, 270%, 271%, 272%, 273%, 274%, 275%, 276%, 277%, 278%, 279%, 280%, 281%, 282%, 283%, 284%, 285%, 286%, 287%, 288%, 289%, 290%, 291%, 292%, 293%, 294%, 295%, 296%, 297%, 298%, 299%, 300%, 301%, 302%, 303%, 304%, 305%, 306%, 307%, 308%, 309%, 310%, 311%, 312%, 313%, 314%, 315%, 316%, 317%, 318%, 319%, 320%, 321%, 322%, 323%, 324%, 325%, 326%, 327%, 328%, 329%, 330%, 331%, 332%, 333%, 334%, 335%, 336%, 337%, 338%, 339%, 340%, 341%, 342%, 343%, 344%, 345%, 346%, 347%, 348%, 349%, 350%, 351%, 352%, 353%, 354%, 355%, 356%, 357%, 358%, 359%, 360%, 361%, 362%, 363%, 364%, 365%, 366%, 367%, 368%, 369%, 370%, 371%, 372%, 373%, 374%, 375%, 376%, 377%, 378%, 379%, 380%, 381%, 382%, 383%, 384%, 385%, 386%, 387%, 388%, 389%, 390%, 391%, 392%, 393%, 394%, 395%, 396%, 397%, 398%, 399%, 400%, 401%, 402%, 403%, 404%, 405%, 406%, 407%, 408%, 409%, 410%, 411%, 412%, 413%, 414%, 415%, 416%, 417%, 418%, 419%, 420%, 421%, 422%, 423%, 424%, 425%, 426%, 427%, 428%, 429%, 430%, 431%, 432%, 433%, 434%, 435%, 436%, 437%, 438%, 439%, 440%, 441%, 442%, 443%, 444%, 445%, 446%, 447%, 448%, 449%, 450%, 451%, 452%, 453%, 454%, 455%, 456%, 457%, 458%, 459%, 460%, 461%, 462%, 463%, 464%, 465%, 466%, 467%, 468%, 469%, 470%, 471%, 472%, 473%, 474%, 475%, 476%, 477%, 478%, 479%, 480%, 481%, 482%, 483%, 484%, 485%, 486%, 487%, 488%, 489%, 490%, 491%, 492%, 493%, 494%, 495%, 496%, 497%, 498%, 499%, 500%, 501%, 502%, 503%, 504%, 505%, 506%, 507%, 508%, 509%, 510%, 511%, 512%, 513%, 514%, 515%, 516%, 517%, 518%, 519%, 520%, 521%, 522%, 523%, 524%, 525%, 526%, 527%, 528%, 529%, 530%, 531%, 532%, 533%, 534%, 535%, 536%, 537%, 538%, 539%, 540%, 541%, 542%, 543%, 544%, 545%, 546%, 547%, 548%, 549%, 550%, 551%, 552%, 553%, 554%, 555%, 556%, 557%, 558%, 559%, 560%, 561%, 562%, 563%, 564%, 565%, 566%, 567%, 568%, 569%, 570%, 571%, 572%, 573%, 574%, 575%, 576%, 577%, 578%, 579%, 580%, 581%, 582%, 583%, 584%, 585%, 586%, 587%, 588%, 589%, 590%, 591%, 592%, 593%, 594%, 595%, 596%, 597%, 598%, 599%, 600%, 601%, 602%, 603%, 604%, 605%, 606%, 607%, 608%, 609%, 610%, 611%, 612%, 613%, 614%, 615%, 616%, 617%, 618%, 619%, 620%, 621%, 622%, 623%, 624%, 625%, 626%, 627%, 628%, 629%, 630%, 631%, 632%, 633%, 634%, 635%, 636%, 637%, 638%, 639%, 640%, 641%, 642%, 643%, 644%, 645%, 646%, 647%, 648%, 649%, 650%, 651%, 652%, 653%, 654%, 655%, 656%, 657%, 658%, 659%, 660%, 661%, 662%, 663%, 664%, 665%, 666%, 667%, 668%, 669%, 670%, 671%, 672%, 673%, 674%, 675%, 676%, 677%, 678%, 679%, 680%, 681%, 682%, 683%, 684%, 685%, 686%, 687%, 688%, 689%, 690%, 691%, 692%, 693%, 694%, 695%, 696%, 697%, 698%, 699%, 700%, 701%, 702%, 703%, 704%, 705%, 706%, 707%, 708%, 709%, 710%, 711%, 712%, 713%, 714%, 715%, 716%, 717%, 718%, 719%, 720%, 721%, 722%, 723%, 724%, 725%, 726%, 727%, 728%, 729%, 730%, 731%, 732%, 733%, 734%, 735%, 736%, 737%, 738%, 739%, 740%, 741%, 742%, 743%, 744%, 745%, 746%, 747%, 748%, 749%, 750%, 751%, 752%, 753%, 754%, 755%, 756%, 757%, 758%, 759%, 760%, 761%, 762%, 763%, 764%, 765%, 766%, 767%, 768%, 769%, 770%, 771%, 772%, 773%, 774%, 775%, 776%, 777%, 778%, 779%, 780%, 781%, 782%, 783%, 784%, 785%, 786%, 787%, 788%, 789%, 790%, 791%, 792%, 793%, 794%, 795%, 796%, 797%, 798%, 799%, 800%, 801%, 802%, 803%, 804%, 805%, 806%, 807%, 808%, 809%, 810%, 811%, 812%, 813%, 814%, 815%, 816%, 817%, 818%, 819%, 820%, 821%, 822%, 823%, 824%, 825%, 826%, 827%, 828%, 829%, 830%, 831%, 832%, 833%, 834%, 835%, 836%, 837%, 838%, 839%, 840%, 841%, 842%, 843%, 844%, 845%, 846%, 847%, 848%, 849%, 850%, 851%, 852%, 853%, 854%, 855%, 856%, 857%, 858%, 859%, 860%, 861%, 862%, 863%, 864%, 865%, 866%, 867%, 868%, 869%, 870%, 871%, 872%, 873%, 874%, 875%, 876%, 877%, 878%, 879%, 880%, 881%, 882%, 883%, 884%, 885%, 886%, 887%, 888%, 889%, 890%, 891%, 892%, 893%, 894%, 895%, 896%, 897%, 898%, 899%, 900%, 901%, 902%, 903%, 904%, 905%, 906%, 907%, 908%, 909%, 910%, 911%, 912%, 913%, 914%, 915%, 916%, 917%, 918%, 919%, 920%, 921%, 922%, 923%, 924%, 925%, 926%, 927%, 928%, 929%, 930%, 931%, 932%, 933%, 934%, 935%, 936%, 937%, 938%, 939%, 940%, 941%, 942%, 943%, 944%, 945%, 946%, 947%, 948%, 949%, 950%, 951%, 952%, 953%, 954%, 955%, 956%, 957%, 958%, 959%, 960%, 961%, 962%, 963%, 964%, 965%, 966%, 967%, 968%, 969%, 970%, 971%, 972%, 973%, 974%, 975%, 976%, 977%, 978%, 979%, 980%, 981%, 982%, 983%, 984%, 985%, 986%, 987%, 988%, 989%, 990%, 991%, 992%, 993%, 994%, 995%, 996%, 997%, 998%, 999%, 1000% or more as compared to cells not in areas or having concentrated ATP or not having FcRN.

### Effective Amounts

In some embodiments, the amount of the primary active agent (e.g., milk exosome, cargo loaded milk exosome, and/or cargo) and/or optional secondary agent can be an effective amount, least effective amount, and/or therapeutically effective amount. As used herein, “effective amount” refers to the amount of the primary and/or optional secondary agent included in the pharmaceutical formulation that achieve one or more therapeutic effects or desired effect. As used herein, “least effective” amount refers to the lowest amount of the primary and/or optional secondary agent that achieves the one or more therapeutic or other desired effects. As used herein, “therapeutically effective amount” refers to the amount of the primary and/or optional secondary agent included in the pharmaceutical formulation that achieves one or more therapeutic effects.

The effective amount, least effective amount, and/or therapeutically effective amount of the primary and optional secondary active agent described elsewhere herein contained in the pharmaceutical formulation can be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges.

In some embodiments, the effective amount, least effective amount, and/or therapeutically effective amount can be an effective concentration, least effective concentration, and/or therapeutically effective concentration, which can each be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, μM, mM, or M or be any numerical value or subrange within any of these ranges.

In other embodiments, the effective amount, least effective amount, and/or therapeutically effective amount of the primary and optional secondary active agent be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value or subrange within any of these ranges.

In some embodiments, the primary and/or the optional secondary active agent present in the pharmaceutical formulation can be any non-zero amount ranging from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation or be any numerical value or subrange within any of these ranges.

In some embodiments where a cell or cell population is present in the pharmaceutical formulation (e.g., as a secondary active agent), the effective amount of cells can be any amount ranging from about 1 or 2 cells to 1×10¹/mL, 1×10²⁰/mL or more, such as about 1×10¹/mL, 1×10²/mL, 1×10³/mL, 1×10⁴/mL, 1×10⁵/mL, 1×10⁶/mL, 1×10⁷/mL, 1×10⁸/mL, 1×10⁹/mL, 1×10¹⁰/mL, 1×10¹¹/mL, 1×10¹²/mL, 1×10¹³/mL, 1×10¹⁴/mL, 1×10¹⁵/mL, 1×10¹⁶/mL, 1×10¹⁷/mL, 1×10¹⁸/mL, 1×10¹⁹/mL, to/or about 1×10²⁰/mL or any numerical value or subrange within any of these ranges.

In some embodiments, the amount or effective amount, particularly where an infective particle is being delivered (e.g., a virus or virus like particle as a primary or secondary agent, e.g, as a cargo), the effective amount of virus particles can be expressed as a titer (plaque forming units per unit of volume) or as a MOI (multiplicity of infection). In some embodiments, the effective amount can be about 1×10¹ particles per pL, nL, μL, mL, or L to 1×10²⁰/particles per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×1¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ particles per pL, nL, μL, mL, or L. In some embodiments, the effective titer can be about 1×10¹ transforming units per pL, nL, μL, mL, or L to 1×10²⁰/transforming units per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×1¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ transforming units per pL, nL, μL, mL, or L or any numerical value or subrange within these ranges. In some embodiments, the MOI of the pharmaceutical formulation can range from about 0.1 to 10 or more, such as 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 or more or any numerical value or subrange within these ranges.

In some embodiments, the amount or effective amount of the one or more of the active agent(s) described herein contained in the pharmaceutical formulation can range from about 1 pg/kg to about 10 mg/kg based upon the bodyweight of the subject in need thereof or average bodyweight of the specific patient population to which the pharmaceutical formulation can be administered.

In embodiments where there is a secondary agent contained in the pharmaceutical formulation, the effective amount of the secondary active agent will vary depending on the secondary agent, the primary agent, the administration route, subject age, disease, stage of disease, among other things, which will be one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially with the compound, derivative thereof, or pharmaceutical formulation thereof.

In some embodiments, the effective amount of the secondary active agent, when optionally present, is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total active agents present in the pharmaceutical formulation or any numerical value or subrange within these ranges. In additional embodiments, the effective amount of the secondary active agent is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation or any numerical value or subrange within these ranges.

### Dosage Forms

In some embodiments, the pharmaceutical formulations described herein can be provided in a dosage form. The dosage form can be administered to a subject in need thereof. The dosage form can be effective generate specific concentration, such as an effective concentration, at a given site in the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a pharmaceutical formulation thereof calculated to produce the desired response or responses in association with its administration. In some embodiments, the given site is proximal to the administration site. In some embodiments, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In some embodiments, the milk exosomes described herein can be the ingredient whose release is delayed. In some embodiments the primary active agent is the ingredient whose release is delayed. In some embodiments, an optional secondary agent can be the ingredient whose release is delayed. In some embodiments, the optional secondary agent is not delayed such that it is delivered prior to the primary active agent (e.g., a milk exosome of the present disclosure). Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome. In these embodiments, primary active ingredient(s), and/or optional secondary active ingredient(s), and/or pharmaceutically acceptable salt thereof where appropriate are incorporated into a liposome. In embodiments where the dosage form is a liposome, the pharmaceutical formulation is thus a liposomal formulation. The liposomal formulation can be administered to a subject in need thereof.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, a primary active ingredient, optional secondary active ingredient, and/or pharmaceutically acceptable salt thereof where appropriate can be in a dosage form adapted for inhalation is in a particle-size-reduced form that is obtained or obtainable by micronization. In some embodiments, the particle size of the size reduced (e.g., micronized) compound or salt or solvate thereof, is defined by a D₅₀ value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In some embodiments, the dosage forms are aerosol formulations suitable for administration by inhalation. In some of these embodiments, the aerosol formulation contains a solution or fine suspension of a primary active ingredient, secondary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional secondary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, 3 or more doses are delivered each time. The aerosol formulations can be administered to a subject in need thereof.

For some dosage forms suitable and/or adapted for inhaled administration, the pharmaceutical formulation is a dry powder inhalable-formulations. In addition to a primary active agent, optional secondary active ingredient, and/or pharmaceutically acceptable salt thereof where appropriate, such a dosage form can contain a powder base such as lactose, glucose, trehalose, mannitol, and/or starch. In some of these embodiments, a primary active agent, secondary active ingredient, and/or pharmaceutically acceptable salt thereof where appropriate is in a particle-size reduced form. In further embodiments, a performance modifier, such as L-leucine or another amino acid, cellobiose octaacetate, and/or metals salts of stearic acid, such as magnesium or calcium stearate. In some embodiments, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in some embodiments, from sterile powders, granules, and tablets. The parenteral formulations can be administered to a subject in need thereof.

For some embodiments, the dosage form contains a predetermined amount of a primary active agent, secondary active ingredient, and/or pharmaceutically acceptable salt thereof where appropriate per unit dose. In an embodiment, the predetermined amount of primary active agent, secondary active ingredient, and/or pharmaceutically acceptable salt thereof where appropriate can be an effective amount, a least effect amount, and/or a therapeutically effective amount. In other embodiments, the predetermined amount of a primary active agent, secondary active agent, and/or pharmaceutically acceptable salt thereof where appropriate, can be an appropriate fraction of the effective amount of the active ingredient.

### Co-Therapies and Combination Therapies

In some embodiments, the pharmaceutical formulation(s) described herein are part of a combination treatment or combination therapy. The combination treatment can include the pharmaceutical formulation described herein and an additional treatment modality. The additional treatment modality can be a chemotherapeutic, a biological therapeutic, surgery, radiation, diet modulation, environmental modulation, a physical activity modulation, and combinations thereof.

In some embodiments, the co-therapy or combination therapy can additionally include but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, anti-cancer drugs, immunogens, and any combination thereof.

In some embodiments the combination therapy includes a milk exosome or population thereof of the present disclosure and one or more immunoglobulins. In some embodiments, the one or more immunoglobulins are IgG, IgM, IgA, IgD, IgE, or any combination thereof. In some embodiments, the one or more immunoglobulins are included in the combination therapy such that delivery of the one or more immunoglobulins are delivered to the subject or a target cell thereof prior to delivery of the milk exosomes of the present disclosure.

In some embodiments the combination therapy includes a milk exosome or population thereof of the present disclosure and an agent capable of stimulating ATP release, secretion, and/or production, ATP, ADP, or any combination thereof. In some embodiments, the one or more agents that stimulate ATP release, secretion, and/or production, ATP, ADP, or any combination thereof is included in the combination therapy such that the one or more agents that stimulate ATP release, secretion, and/or production, ATP, ADP, or any combination thereof are delivered to the subject and/or target cell prior to delivery of the milk exosomes of the combination therapy.

In some embodiments, the combination therapy includes a milk exosome or population thereof of the present disclosure and one or more immunoglobulins, one or more agents that stimulating ATP release, secretion, and/or production, ATP, ADP, or any combination thereof. In some embodiments, the one or more immunoglobulins, one or more agents that stimulating ATP release, secretion, and/or production, ATP, ADP, or any combination thereof are included in the combination therapy such that the one or more immunoglobulins, one or more agents that stimulating ATP release, secretion, and/or production, ATP, ADP, or any combination thereof is delivered to the subject in need thereof or a cell target cell prior to delivery of the milk exosomes of the combination therapy.

In some embodiments, the milk exosomes have increased uptake in cells that have concentrated ATP or are in environments with concentrated ATP as compared to cells that do not have concentrated levels of ATP or are in environments without concentrated ATP. In some embodiments, the milk exosomes have increased uptake in cells having FcRN on their surface. gastrointestinal cell, a heart cell, a liver cell, a brain cell, a lung cell, reproductive cell, a nerve cell, a cancer cell, or any combination thereof. In some embodiments, the cell is a polar cell, meaning that the cells have a different apical and basal membrane surface. In some embodiments, the cell is an enterocyte. In some embodiments, the cells is a blood-brain barrier cell. In some embodiments, uptake is increased by the combination therapy by 0.01%, to/or 0.02%, 0.03%, 0.04%, 0.05%, 0.06%, 0.07%, 0.08%, 0.09%, 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.31%, 0.32%, 0.33%, 0.34%, 0.35%, 0.36%, 0.37%, 0.38%, 0.39%, 0.4%, 0.41%, 0.42%, 0.43%, 0.44%, 0.45%, 0.46%, 0.47%, 0.48%, 0.49%, 0.5%, 0.51%, 0.52%, 0.53%, 0.54%, 0.55%, 0.56%, 0.57%, 0.58%, 0.59%, 0.6%, 0.61%, 0.62%, 0.63%, 0.64%, 0.65%, 0.66%, 0.67%, 0.68%, 0.69%, 0.7%, 0.71%, 0.72%, 0.73%, 0.74%, 0.75%, 0.76%, 0.77%, 0.78%, 0.79%, 0.8%, 0.81%, 0.82%, 0.83%, 0.84%, 0.85%, 0.86%, 0.87%, 0.88%, 0.89%, 0.9%, 0.91%, 0.92%, 0.93%, 0.94%, 0.95%, 0.96%, 0.97%, 0.98%, 0.99%1, 1%, 2%, 3%, 4%, 5%, 6%, 7%, 8%, 9% 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 101%, 102%, 103%, 104%, 105%, 106%, 107%, 108%, 109%, O110%, 111%, 112%, 113%, 114%, 115%, 116%, 117%, 118%, 119%, 120%, 121%, 122%, 123%, 124%, 125%, 126%, 127%, 128%, 129%, 130%, 131%, 132%, 133%, 134%, 135%, 136%, 137%, 138%, 139%, 140%, 141%, 142%, 143%, 144%, 145%, 146%, 147%, 148%, 149%, 150%, 151%, 152%, 153%, 154%, 155%, 156%, 157%, 158%, 159%, 160%, 161%, 162%, 163%, 164%, 165%, 166%, 167%, 168%, 169%, 170%, 171%, 172%, 173%, 174%, 175%, 176%, 177%, 178%, 179%, 180%, 181%, 182%, 183%, 184%, 185%, 186%, 187%, 188%, 189%, 190%, 191%, 192%, 193%, 194%, 195%, 196%, 197%, 198%, 199%, 200%, 201%, 202%, 203%, 204%, 205%, 206%, 207%, 208%, 209%, 210%, 211%, 212%, 213%, 214%, 215%, 216%, 217%, 218%, 219%, 220%, 221%, 222%, 223%, 224%, 225%, 226%, 227%, 228%, 229%, 230%, 231%, 232%, 233%, 234%, 235%, 236%, 237%, 238%, 239%, 240%, 241%, 242%, 243%, 244%, 245%, 246%, 247%, 248%, 249%, 250%, 251%, 252%, 253%, 254%, 255%, 256%, 257%, 258%, 259%, 260%, 261%, 262%, 263%, 264%, 265%, 266%, 267%, 268%, 269%, 270%, 271%, 272%, 273%, 274%, 275%, 276%, 277%, 278%, 279%, 280%, 281%, 282%, 283%, 284%, 285%, 286%, 287%, 288%, 289%, 290%, 291%, 292%, 293%, 294%, 295%, 296%, 297%, 298%, 299%, 300%, 301%, 302%, 303%, 304%, 305%, 306%, 307%, 308%, 309%, 310%, 311%, 312%, 313%, 314%, 315%, 316%, 317%, 318%, 319%, 320%, 321%, 322%, 323%, 324%, 325%, 326%, 327%, 328%, 329%, 330%, 331%, 332%, 333%, 334%, 335%, 336%, 337%, 338%, 339%, 340%, 341%, 342%, 343%, 344%, 345%, 346%, 347%, 348%, 349%, 350%, 351%, 352%, 353%, 354%, 355%, 356%, 357%, 358%, 359%, 360%, 361%, 362%, 363%, 364%, 365%, 366%, 367%, 368%, 369%, 370%, 371%, 372%, 373%, 374%, 375%, 376%, 377%, 378%, 379%, 380%, 381%, 382%, 383%, 384%, 385%, 386%, 387%, 388%, 389%, 390%, 391%, 392%, 393%, 394%, 395%, 396%, 397%, 398%, 399%, 400%, 401%, 402%, 403%, 404%, 405%, 406%, 407%, 408%, 409%, 410%, 411%, 412%, 413%, 414%, 415%, 416%, 417%, 418%, 419%, 420%, 421%, 422%, 423%, 424%, 425%, 426%, 427%, 428%, 429%, 430%, 431%, 432%, 433%, 434%, 435%, 436%, 437%, 438%, 439%, 440%, 441%, 442%, 443%, 444%, 445%, 446%, 447%, 448%, 449%, 450%, 451%, 452%, 453%, 454%, 455%, 456%, 457%, 458%, 459%, 460%, 461%, 462%, 463%, 464%, 465%, 466%, 467%, 468%, 469%, 470%, 471%, 472%, 473%, 474%, 475%, 476%, 477%, 478%, 479%, 480%, 481%, 482%, 483%, 484%, 485%, 486%, 487%, 488%, 489%, 490%, 491%, 492%, 493%, 494%, 495%, 496%, 497%, 498%, 499%, 500%, 501%, 502%, 503%, 504%, 505%, 506%, 507%, 508%, 509%, 510%, 511%, 512%, 513%, 514%, 515%, 516%, 517%, 518%, 519%, 520%, 521%, 522%, 523%, 524%, 525%, 526%, 527%, 528%, 529%, 530%, 531%, 532%, 533%, 534%, 535%, 536%, 537%, 538%, 539%, 540%, 541%, 542%, 543%, 544%, 545%, 546%, 547%, 548%, 549%, 550%, 551%, 552%, 553%, 554%, 555%, 556%, 557%, 558%, 559%, 560%, 561%, 562%, 563%, 564%, 565%, 566%, 567%, 568%, 569%, 570%, 571%, 572%, 573%, 574%, 575%, 576%, 577%, 578%, 579%, 580%, 581%, 582%, 583%, 584%, 585%, 586%, 587%, 588%, 589%, 590%, 591%, 592%, 593%, 594%, 595%, 596%, 597%, 598%, 599%, 600%, 601%, 602%, 603%, 604%, 605%, 606%, 607%, 608%, 609%, 610%, 611%, 612%, 613%, 614%, 615%, 616%, 617%, 618%, 619%, 620%, 621%, 622%, 623%, 624%, 625%, 626%, 627%, 628%, 629%, 630%, 631%, 632%, 633%, 634%, 635%, 636%, 637%, 638%, 639%, 640%, 641%, 642%, 643%, 644%, 645%, 646%, 647%, 648%, 649%, 650%, 651%, 652%, 653%, 654%, 655%, 656%, 657%, 658%, 659%, 660%, 661%, 662%, 663%, 664%, 665%, 666%, 667%, 668%, 669%, 670%, 671%, 672%, 673%, 674%, 675%, 676%, 677%, 678%, 679%, 680%, 681%, 682%, 683%, 684%, 685%, 686%, 687%, 688%, 689%, 690%, 691%, 692%, 693%, 694%, 695%, 696%, 697%, 698%, 699%, 700%, 701%, 702%, 703%, 704%, 705%, 706%, 707%, 708%, 709%, 710%, 711%, 712%, 713%, 714%, 715%, 716%, 717%, 718%, 719%, 720%, 721%, 722%, 723%, 724%, 725%, 726%, 727%, 728%, 729%, 730%, 731%, 732%, 733%, 734%, 735%, 736%, 737%, 738%, 739%, 740%, 741%, 742%, 743%, 744%, 745%, 746%, 747%, 748%, 749%, 750%, 751%, 752%, 753%, 754%, 755%, 756%, 757%, 758%, 759%, 760%, 761%, 762%, 763%, 764%, 765%, 766%, 767%, 768%, 769%, 770%, 771%, 772%, 773%, 774%, 775%, 776%, 777%, 778%, 779%, 780%, 781%, 782%, 783%, 784%, 785%, 786%, 787%, 788%, 789%, 790%, 791%, 792%, 793%, 794%, 795%, 796%, 797%, 798%, 799%, 800%, 801%, 802%, 803%, 804%, 805%, 806%, 807%, 808%, 809%, 810%, 811%, 812%, 813%, 814%, 815%, 816%, 817%, 818%, 819%, 820%, 821%, 822%, 823%, 824%, 825%, 826%, 827%, 828%, 829%, 830%, 831%, 832%, 833%, 834%, 835%, 836%, 837%, 838%, 839%, 840%, 841%, 842%, 843%, 844%, 845%, 846%, 847%, 848%, 849%, 850%, 851%, 852%, 853%, 854%, 855%, 856%, 857%, 858%, 859%, 860%, 861%, 862%, 863%, 864%, 865%, 866%, 867%, 868%, 869%, 870%, 871%, 872%, 873%, 874%, 875%, 876%, 877%, 878%, 879%, 880%, 881%, 882%, 883%, 884%, 885%, 886%, 887%, 888%, 889%, 890%, 891%, 892%, 893%, 894%, 895%, 896%, 897%, 898%, 899%, 900%, 901%, 902%, 903%, 904%, 905%, 906%, 907%, 908%, 909%, 910%, 911%, 912%, 913%, 914%, 915%, 916%, 917%, 918%, 919%, 920%, 921%, 922%, 923%, 924%, 925%, 926%, 927%, 928%, 929%, 930%, 931%, 932%, 933%, 934%, 935%, 936%, 937%, 938%, 939%, 940%, 941%, 942%, 943%, 944%, 945%, 946%, 947%, 948%, 949%, 950%, 951%, 952%, 953%, 954%, 955%, 956%, 957%, 958%, 959%, 960%, 961%, 962%, 963%, 964%, 965%, 966%, 967%, 968%, 969%, 970%, 971%, 972%, 973%, 974%, 975%, 976%, 977%, 978%, 979%, 980%, 981%, 982%, 983%, 984%, 985%, 986%, 987%, 988%, 989%, 990%, 991%, 992%, 993%, 994%, 995%, 996%, 997%, 998%, 999%, 1000% or more as compared to cells not in areas or having concentrated ATP or not having FcRN.

### Administration of the Pharmaceutical Formulations

The pharmaceutical formulations or dosage forms thereof described herein can be administered one or more times hourly, daily, monthly, or yearly (e.g., 1, 2, 3, 4, 5, 6, 7, 8, 9, 10, 11, 12, 13, 14, 15, 16, 17, 18, 19, 20, or more times hourly, daily, monthly, or yearly). In some embodiments, the pharmaceutical formulations or dosage forms thereof described herein can be administered continuously over a period of time ranging from minutes to hours to days. Devices and dosages forms are known in the art and described herein that are effective to provide continuous administration of the pharmaceutical formulations described herein. In some embodiments, the first one or a few initial amount(s) administered can be a higher dose than subsequent doses. This is typically referred to in the art as a loading dose or doses and a maintenance dose, respectively. In some embodiments, the pharmaceutical formulations can be administered such that the doses over time are tapered (increased or decreased) overtime so as to wean a subject gradually off of a pharmaceutical formulation or gradually introduce a subject to the pharmaceutical formulation.

As previously discussed, the pharmaceutical formulation can contain a predetermined amount of a primary active agent, secondary active agent, and/or pharmaceutically acceptable salt thereof where appropriate. In some of these embodiments, the predetermined amount can be an appropriate fraction of the effective amount of the active ingredient. Such unit doses may therefore be administered once or more than once a day, month, or year (e.g., 1, 2, 3, 4, 5, 6, or more times per day, month, or year). Such pharmaceutical formulations may be prepared by any of the methods well known in the art.

Where co-therapies or multiple pharmaceutical formulations are to be delivered to a subject, the different therapies or formulations can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g., within seconds or just a few minutes apart), where the intent is that the formulations be administered together at the same time.

### Kits

Any of the compounds, compositions, formulations, end/or exosomes, described herein or a combination thereof can be presented as a combination kit. In some embodiments, the kit includes one or more filters, tubes, devices, etc. that is used to prepare milk exosomes according to a method described herein. As used herein, the terms “combination kit” or “kit of parts” refers to the compounds, compositions, formulations, particles, cells and any additional components that are used to package, sell, market, deliver, and/or administer the combination of elements or a single element, such as the active ingredient, contained therein. Such additional components include, but are not limited to, packaging, syringes, blister packages, bottles, and the like. When one or more of the compounds, compositions, formulations, particles, cells, described herein or a combination thereof (e.g., agents) contained in the kit are administered simultaneously, the combination kit can contain the active agents in a single formulation, such as a pharmaceutical formulation, (e.g., a tablet) or in separate formulations. When the compounds, compositions, formulations, particles, and cells described herein or a combination thereof and/or kit components are not administered simultaneously, the combination kit can contain each agent or other component in separate pharmaceutical formulations. The separate kit components can be contained in a single package or in separate packages within the kit.

In some embodiments, the combination kit also includes instructions printed on or otherwise contained in a tangible medium of expression. The instructions can provide information regarding the content of the compounds, compositions, formulations, particles, and/or exosomes described herein or any combination thereof contained therein, safety information regarding the content of the compounds, compositions, formulations (e.g., pharmaceutical formulations), particles, and/or exosomes described herein or a combination thereof contained therein, information regarding the dosages, indications for use, and/or recommended treatment regimen(s) for the compound(s) and/or pharmaceutical formulations contained therein. In some embodiments, the instructions provide direction on how to prepare milk exosomes according to a method described elsewhere herein. In some embodiments, the instructions can provide directions for administering the compounds, compositions, formulations, particles, and cells described herein or a combination thereof to a subject in need thereof.

### Methods of Using the Targeted Milk Exosomes

Described in certain embodiments herein are methods of treating an injury at an injury site or treating a cancer in a subject in need thereof comprising administering to the subject in need thereof, a milk exosome of the present disclosure described herein or a population thereof or a pharmaceutical formulation comprising the milk exosome of the present disclosure described herein or the population thereof. Without being bound by theory, in some embodiments, the bioavailability of an exogenous cargo of the milk exosome(s) of the present disclosure can be increased over conventional delivery approaches. In some embodiments, the mammalian milk exosome is a bovid milk exosome, an ovid milk exosome, a caprine milk exosome, a camelid milk exosome, or a human milk exosome. In certain example embodiments, the milk exosome is a bovine milk exosome. The milk exosomes can be isolated from any suitable mammalian milk source. Milk exosomes are as described elsewhere herein.

In certain example embodiments, the method further comprises administering an amount of one or more immunoglobulins to the subject in need thereof. In certain example embodiments, administering the amount of one or more immunoglobulins to subject in need thereof occurs prior to administering the amount of a milk exosome of claim of the present disclosure or a population thereof or a pharmaceutical formulation comprising the milk exosome of the present disclosure described herein or the population thereof. In certain example embodiments, the one or more immunoglobulins are selected from are selected from IgG, IgM, IgA, IgD, and/or IgE. In certain example embodiments, the one or more immunoglobulins consists of or comprises IgG. In certain example embodiments, the one or more immunoglobulins increase uptake of the milk exosome or population thereof in a cell. In certain example embodiments, the cell is a polarized cell. In certain example embodiments, the cell is a gastrointestinal cell, a heart cell, a liver cell, brain cell, nerve cell, cancer cell, or any combination thereof. In some embodiments, the cell is an enterocyte or a blood-brain barrier cell. In some embodiments, the immunoglobulin(s) are delivered 1-100 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, to/or 100) seconds, minutes, hours, or days prior to delivery of the milk exosomes. In some embodiments, the milk exosomes and the immunoglobulin(s) are delivered simultaneously. In some embodiments, the milk exosomes and the immunoglobulin(s) are delivered simultaneously, but the immunoglobulin(s) and/or milk exosomes are in dosage forms that delays the release of the milk exosomes and/or accelerates the release of the immunoglobulin(s) such that the immunoglobulin(s) are delivered first. Delivery of the milk exosomes and/or immunoglobulin(s) can occur 1-100 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, to/or 100) times per day, month, or year.

In certain example embodiments, the method further comprises administering one or more agents that stimulate ATP release, secretion, and/or production, ATP, ADP, or any combination thereof to the subject in need thereof. In certain example embodiments, administering the one or more agents that stimulate ATP release, secretion, and/or production, ATP, ADP, or any combination thereof to subject in need thereof occurs prior to administering the amount of a milk exosome of the present disclosure described herein or a population thereof or a pharmaceutical formulation comprising the milk exosome of the present disclosure described herein or the population thereof. In certain example embodiments, the one or more agents that stimulate ATP release, secretion, and/or production, ATP, ADP, or any combination thereof increases the cellular uptake of the milk exosome or population thereof in a cell. In certain example embodiments, the cell is a polarized cell. In certain example embodiments, the cell is a gastrointestinal cell, heart cell, liver cell, brain cell, nerve cell, cancer cell, or any combination thereof. In some embodiments, the cell is an enterocyte or a blood-brain barrier cell. In some embodiments, the one or more agents that stimulate ATP release, secretion, and/or production, ATP, ADP, or any combination thereof are delivered 1-100 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, to/or 100) seconds, minutes, hours, or days prior to delivery of the milk exosomes. In some embodiments, the milk exosomes and the one or more agents that stimulate ATP release, secretion, and/or production, ATP, ADP, or any combination thereof are delivered simultaneously. In some embodiments, the milk exosomes and the one or more agents that stimulate ATP release, secretion, and/or production, ATP, ADP, or any combination thereof are delivered simultaneously, but the immunoglobulin(s) and/or milk exosomes are in dosage forms that delays the release of the milk exosomes and/or accelerates the release of the one or more agents that stimulate ATP release, secretion, and/or production, ATP, ADP, or any combination thereof that the immunoglobulin(s) are delivered first. Delivery of the milk exosomes and/or one or more agents that stimulate ATP release, secretion, and/or production, ATP, ADP, or any combination thereof can occur 1-100 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, to/or 100) times per day, month, or year.

In certain example embodiments, the injury site is a mechanical injury site or a non-mechanical injury site, radiation injury, inflammatory injury, or an ischemic injury. In certain example embodiments, the non-mechanical injury is a chemical injury, electrical injury, radiation injury, or an ischemic injury. In some embodiments the subject in need thereof has a disease, disorder or condition. In some embodiments, the disease, disorder, or condition results in injured cells or areas of inflammation. Exemplary diseases or disorders include, but are not limited to, a cancer, a viral infection, a bacterial infection, a parasite infection, a mechanical injury (e.g., external and internal wounds and tissue injuries, cuts, scrapes, burns, surgical wounds, wounds secondary to infection, disease, or condition, etc.), cancer, non-mechanical injury (e.g., chemical injury, electrical injury, radiation injury, ischemic and/or hypoxic injuries (e.g. myocardial infarction, ischemic wounds and/or stroke), multiple sclerosis, psoriasis, scleroderma, acne, eczema, or a disease of the skin and/or connective tissues, cardiac diseases or disorders, neurodegenerative diseases or disorders, neurological disorders, atherosclerosis, pathologies involving epithelial permeabilization and/or neovascularization (e.g., angiogenesis or vasculogenesis), respiratory distress syndrome (RDS), reperfusion injuries, dermal vascular blemish or malformation, macular degeneration, neovascularization of choriocapillaries through Bruch's membrane, diabetic retinopathy, (inflammatory and inflammation-related diseases and disorders), and radiation dermatitis.

Wounds can be chronic wounds or wounds that appear to not completely heal. Wounds that have not healed within three months, for example, are said to be chronic. Chronic wounds include, diabetic foot ulcers, ischemic, venous ulcers, venous leg ulcers, venous stasis, arterial, pressure, vasculitic, infectious, decubitis, burn, trauma-induced, gangrenous and mixed ulcers. Chronic wounds include wounds that are characterized by and/or chronic inflammation, deficient and overprofuse granulation tissue differentiation and failure of re-epithelialization and wound closure and longer repair times. Chronic wounds can include ocular ulcers, including corneal ulcers. Use of the disclosed invention in wound healing and tissue regeneration can include in humans and agricultural, sports and pet animals.

Tissue injuries can result from, for example, a cut, scrape, compression wound, stretch injury, laceration wound, crush wound, bite wound, graze, bullet wound, explosion injury, body piercing, stab wound, surgical wound, surgical intervention, medical intervention, host rejection following cell, tissue or organ grafting, pharmaceutical effect, pharmaceutical side-effect, bed sore, radiation injury, radiation illness, cosmetic skin wound, internal organ injury, disease process (e.g., asthma, cancer), infection, infectious agent, developmental process, maturational process (e.g., acne), genetic abnormality, developmental abnormality, environmental toxin, allergen, scalp injury, facial injury, jaw injury, sex organ injury, joint injury, excretory organ injury, foot injury, finger injury, toe injury, bone injury, eye injury, corneal injury, muscle injury, adipose tissue injury, lung injury, airway injury, hernia, anus injury, piles, ear injury, skin injury, abdominal injury, retinal injury, eye injury, corneal injury, arm injury, leg injury, athletic injury, back injury, birth injury, premature birth injury, toxic bite, sting, injury to barrier function, injury to endothelial barrier function, injury to epithelial barrier function, tendon injury, ligament injury, heart injury, heart valve injury, vascular system injury, cartilage injury, lymphatic system injury, craniocerebral trauma, dislocation, esophageal perforation, fistula, nail injury, foreign body, fracture, frostbite, hand injury, heat stress disorder, laceration, neck injury, self-mutilation, shock, traumatic soft tissue injury, spinal cord injury, spinal injury, sprain, strain, tendon injury, ligament injury, cartilage injury, thoracic injury, tooth injury, trauma, nervous system injury, burn, burn wound, wind burn, sun burn, chemical burn, aging, aneurism, stroke, surgical radiation injury, digestive tract injury, infarct, or ischemic injury.

Cardiac diseases and disorders can include, but are not limited to, myocardial infarction, cardio myopathies (e.g., hypertrophic cardiomyopathy), arrhythmias, congestive heart failure. The regenerative effects of the provided composition may result in beneficial changes in membrane excitability and ion transients of the heart. There are many different types of arrhythmia that can lead to abnormal function in the human heart. Arrhythmias include, but are not limited to bradycardias, tachycardias, alternans, automaticity defects, reentrant arrhythmias, fibrillation, AV nodal arrhythmias, atrial arrhythmias and triggered beats, Long QT syndrome, Short QT syndrome, Brugada syndrome, premature atrial Contractions, wandering Atrial pacemaker, Multifocal atrial tachycardia, Atrial flutter, Atrial fibrillation, Supraventricular tachycardia, AV nodal reentrant tachycardia is the most common cause of Paroxysmal Supraventricular Tachycardia, Junctional rhythm, Junctional tachycardia, Premature junctional complex, Wolff-Parkinson-White syndrome, Lown-Ganong-Levine syndrome, Premature Ventricular Contractions (PVC) sometimes called Ventricular Extra Beats, alternans and discordant alternans, Accelerated idioventricular rhythm, Monomorphic Ventricular tachycardia, Polymorphic ventricular tachycardia, Ventricular fibrillation, First degree heart block, which manifests as PR prolongation, Second degree heart block, Type 1 Second degree heart block, Type 2 Second degree heart block, Third degree heart block, and several accessory pathway disorders (e.g., Wolff-Parkinson-White syndrome (WPW)).

Neurodegenerative and neurological disorders include, but are not limited to dementia, Alzheimer's disease, Parkinson's disease and related PD-diseases, amyotrophic lateral sclerosis (ALS), motor neuron disease, schizophrenia, spinocerebellar ataxia, prion disease, Spinal muscular atrophy (SMA), multiple sclerosis, epilepsy and other seizure disorders, and Huntington's disease.

Inflammatory diseases and inflammatory-related diseases and disorders can be asthma, eczema, sinusitis, atherosclerosis, arthritis (including but not limited to rheumatoid arthritis), inflammatory bowel disease, cutaneous and systemic mastocytosis, psoriasis, and multiple sclerosis. As used herein, the term “inflammatory disorder” can include diseases or disorders which are caused, at least in part, or exacerbated, by inflammation, which is generally characterized by increased blood flow, edema, activation of immune cells (e.g., proliferation, cytokine production, or enhanced phagocytosis), heat, redness, swelling, pain and/or loss of function in the affected tissue or organ. The cause of inflammation can be due to physical damage, chemical substances, micro-organisms, tissue necrosis, cancer, or other agents or conditions.

Inflammatory disorders include acute inflammatory disorders, chronic inflammatory disorders, and recurrent inflammatory disorders. Acute inflammatory disorders are generally of relatively short duration, and last for from about a few minutes to about one to two days, although they can last several weeks. Characteristics of acute inflammatory disorders include increased blood flow, exudation of fluid and plasma proteins (edema) and emigration of leukocytes, such as neutrophils. Chronic inflammatory disorders, generally, are of longer duration, e.g., weeks to months to years or longer, and are associated histologically with the presence of lymphocytes and macrophages and with proliferation of blood vessels and connective tissue. Recurrent inflammatory disorders include disorders which recur after a period of time or which have periodic episodes. Some inflammatory disorders fall within one or more categories. Exemplary inflammatory disorders include but are not limited to atherosclerosis; arthritis; inflammation-promoted cancers; asthma; autoimmune uveitis; adoptive immune response; dermatitis; multiple sclerosis; diabetic complications; osteoporosis; Alzheimer's disease; cerebral malaria; hemorrhagic fever; autoimmune disorders; and inflammatory bowel disease. In some embodiments, the inflammatory disorder is an autoimmune disorder that, in some embodiments, is selected from lupus, rheumatoid arthritis, and autoimmune encephalomyelitis.

In some embodiments, the inflammatory disorder is a brain-related inflammatory disorder. The term “brain-related inflammatory” disorder is used herein to refer to a subset of inflammatory disorders that are caused, at least in part, or originate or are exacerbated, by inflammation in the brain of a subject.

In some embodiments, the subject in need thereof has cancer.

In certain example embodiments, the milk exosome or population thereof and/or pharmaceutical formulation are irradiated, sterilized, or both.

In certain example embodiments, the milk exosome, population of milk exosomes, or pharmaceutical formulation of the present disclosure described herein, wherein the milk exosome or population thereof and/or pharmaceutical formulation thereof are irradiated, sterilized, or both.

Further embodiments are illustrated in the following Examples which are given for illustrative purposes only and are not intended to limit the scope of the invention.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1—Bovine Milk Derived Extracellular Vesicles as a Cargo Delivery Platform

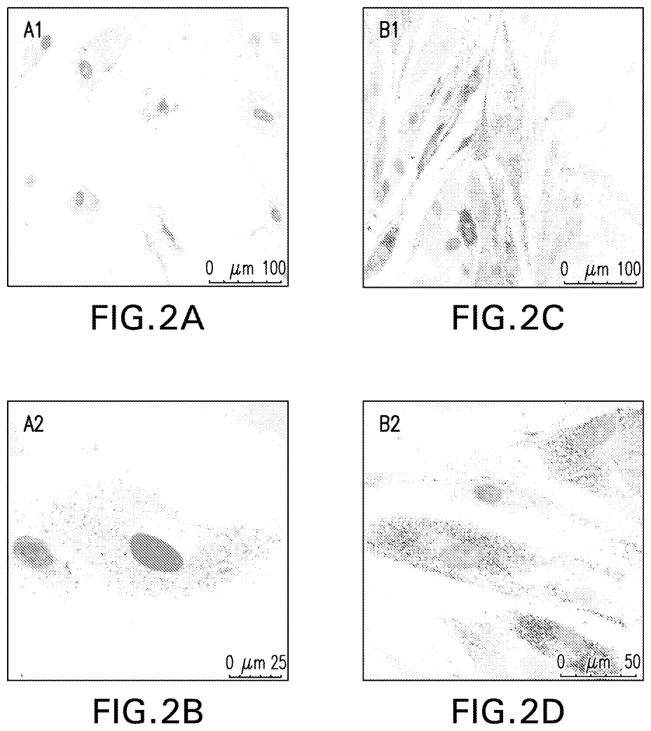

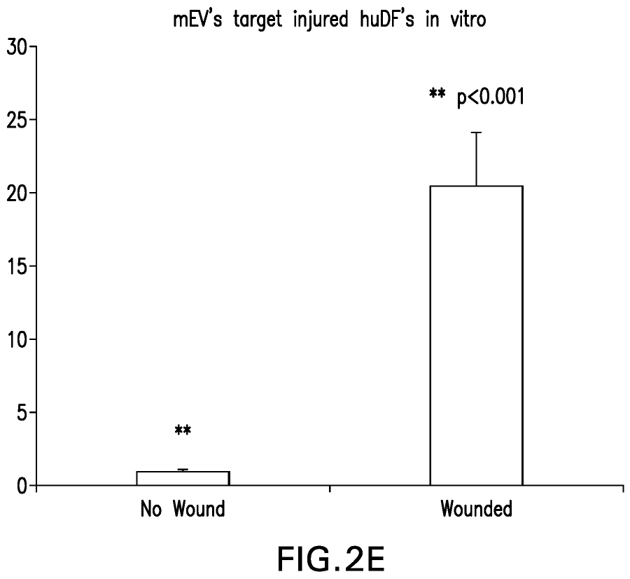

Bovine milk-derived extracellular vesicles (mEV's) can be orally administered, and bypass critical barriers in the body using a protocol described in WO 2022/182782 and administered orally, mEV's target injured tissue in vivo. FIG. 1A-1Q displays fluorescently tagged mEV's experiencing targeting affects to wounded skin (FIG. 1A-1D) and to wounded cardiac tissue (FIG. 1I-1L), while not showing a propensity for accumulation in non-injured skin (FIG. 1E-1H) or cardiac (FIG. 1M-1O) tissues. Particle count analysis was performed on each skin (FIG. 1P) and cardiac tissue (FIG. 1Q), showing statistically significant increases in uptake in injured tissues—greater than 30× expression in ischemic cardiac tissue and nearly 10× in a dermal injury model. To follow up in vivo experiments, huDFs were injured with a scratch wound and fluorescently tagged mEV's were then administered (FIG. 2A-2E). Wounding in vitro causes a 20× enhancement in mEV uptake (FIG. 2E), as shown by the fluorescent images for unwounded cells (FIG. 2A-2B) versus wounded cells (FIG. 2C-2D).

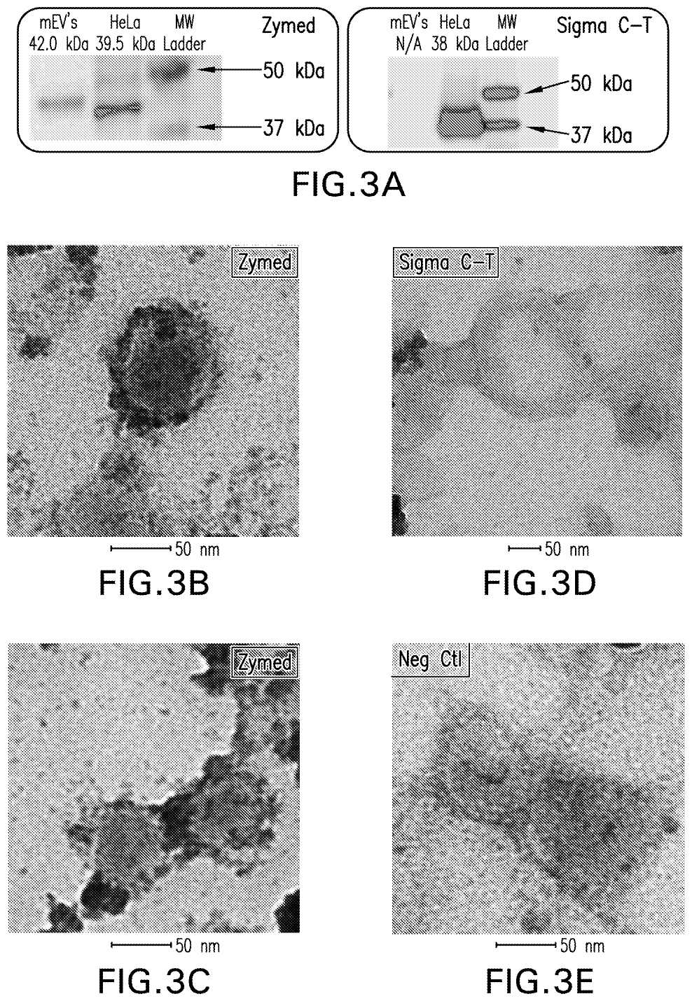

mEV's have previously been shown to contain the gap junctional protein Connexin-43 (Cx43), which has implications in wound healing and cellular communication. To verify that the mEV's, isolated with our unique process, contain Cx43, Applicant performed western blotting for the c-tail fragment as well as the c-terminus fragment (FIG. 3A-3E). Interestingly, Applicant found that the C-tail is present, while the C-terminus is conspicuously absent (FIG. 3A). This analysis was then verified by the use of Nanogold TEM imaging, for C-tail (FIG. 3B), which showed positive expression, while the C-terminus (FIG. 3D) did not. Negative controls for each antibody are shown in FIG. 3C (C-tail) and FIG. 3E (C-terminus). This suggests that while Cx43 is present in mEV's, there is a noticeable truncation of the protein, which, without being bound by theory, is postulated to result in the formation of an endogenous C-terminal fragment held within the mEV's.

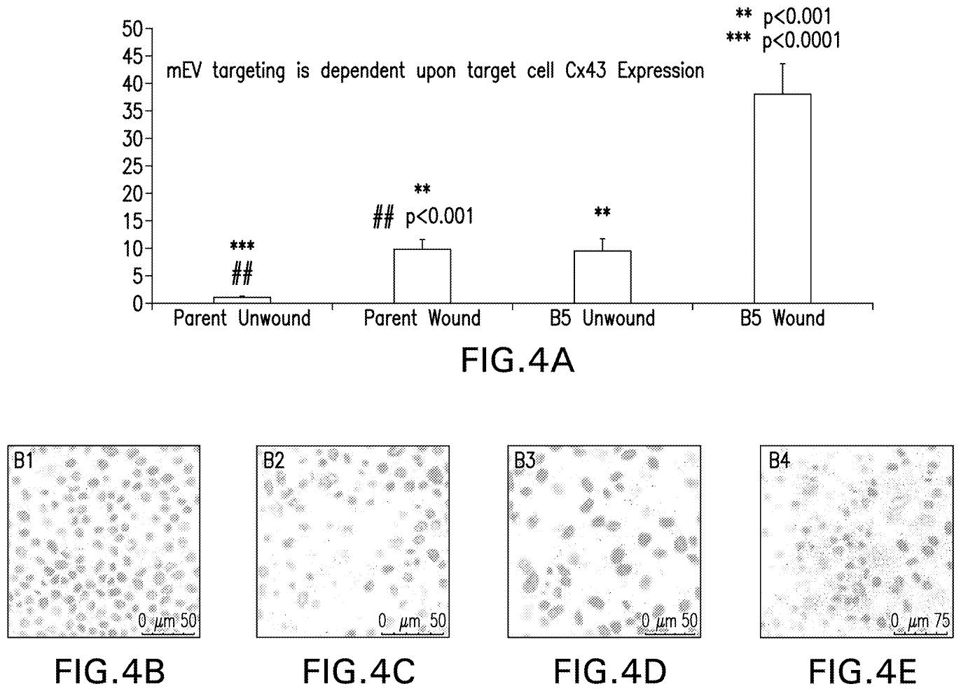

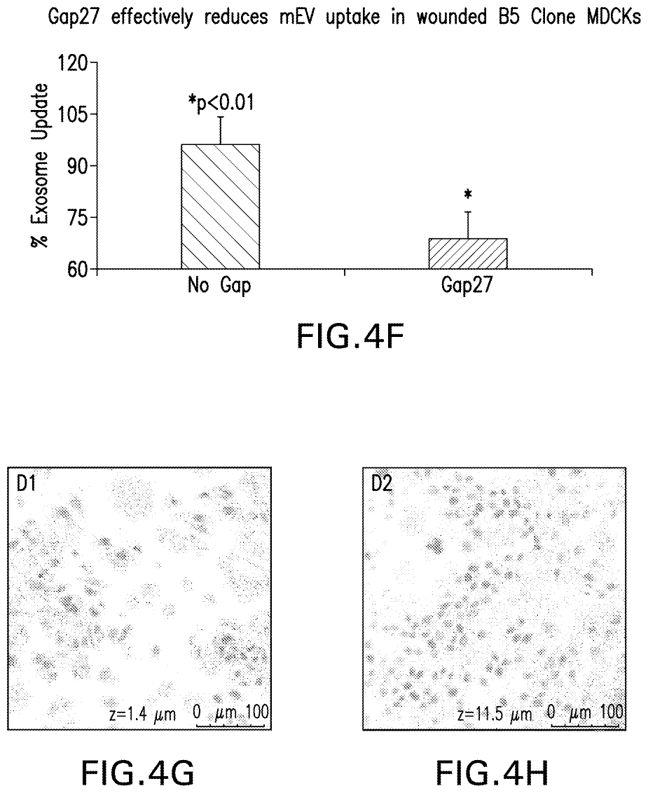

Given the known presence of a truncated form of Cx43, Applicant then performed scratch wound uptake analysis for a series of Madine-Darby Canine Kidney (MDCK) cells (FIG. 4A-4H)—one of which was altered to exhibit increased expression, the B5 Clone line, and one which was altered to exhibit decreased expression, the Parent Lampe line. When methods from the huDF wound experiment were followed exactly the same, the cells reacted very differently, suggesting an innate role of Cx43 in mEV bioactivity. Specifically, when Parent Lampe cells were wounded, mEV uptake only increased slightly, as shown in FIG. 4C-unwounded Parent Lampe cells are shown in FIG. 4B. The associated bar graph for particle count analysis is shown above in FIG. 4A. Meanwhile, when B5 clone line cells were wounded (FIG. 4E), uptake was enhanced over baseline by over 30×—interestingly, B5 clone cells also exhibit enhanced uptake in a non-wounded model (FIG. 4D). This insinuates that mEV uptake by cells is mediated by Cx43 in both unwounded and wounded situations, with unwounded B5 clone cells experiencing similar uptake levels to that of wounded Parent Lampe cells (FIG. 4A). Applicant then tested for the mechanism of uptake in these cells—B5 clone cells were scratch wounded and given fluorescently tagged mEV's, then some were also provided with Gap27 (FIG. 4F), a known Connexin channel blocker. When co-incubated with Gap27 (D2), mEV uptake was blocked by over 25% (FIG. 4F) when compared to those untreated. Without being bound by theory, this suggests a key role of Cx43 in cellular uptake, and by blocking Cx43, we then block a significant portion of mEV uptake in an injured model. Another mechanism of uptake of mEV's in a wounded environment that Applicant has discovered is ATP (FIG. 5A-5C). When B5 Clone cells were wounded and provided mEV's (FIG. 5A), normal wounded uptake was seen; when cells were co-incubated in mEV's with 100 uM apyrase (FIG. 5B), an ATP dephosphorylating agent, cellular uptake was completely blocked, experiencing a >95% reduction in mEV uptake. This suggests a highly critical role of ATP in mEV uptake in wounded environments.

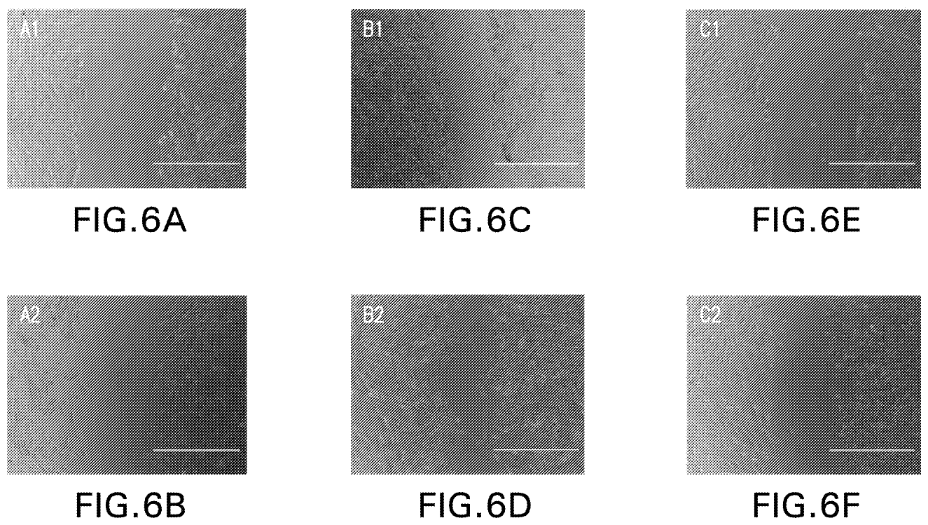

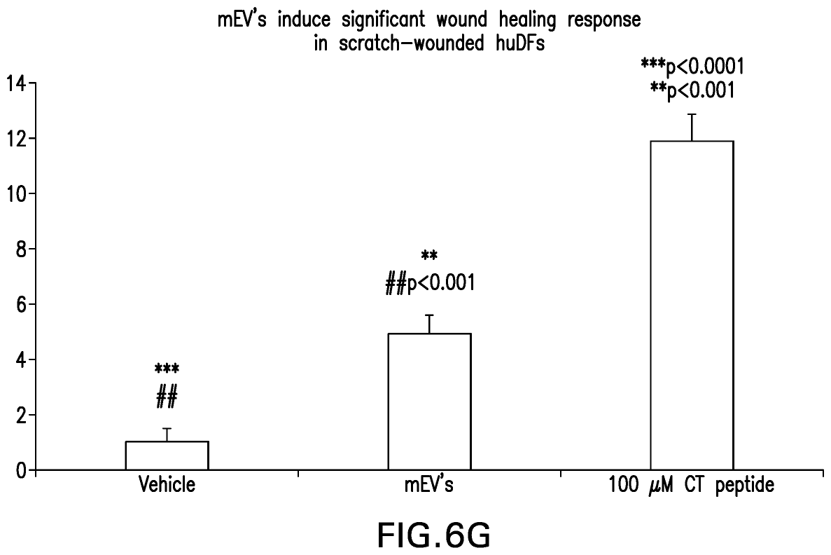

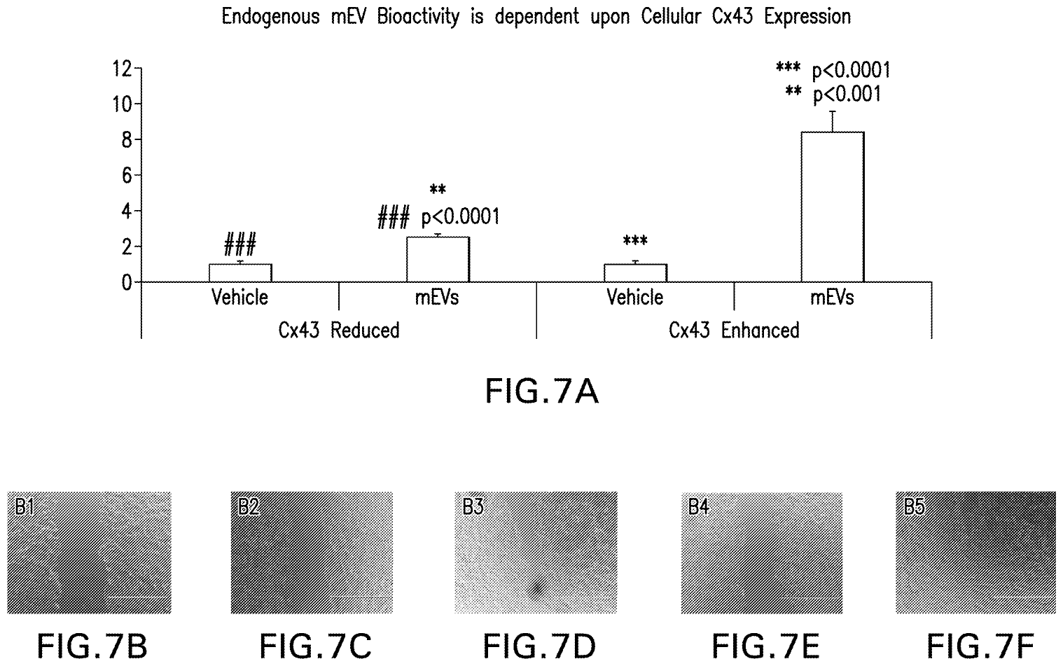

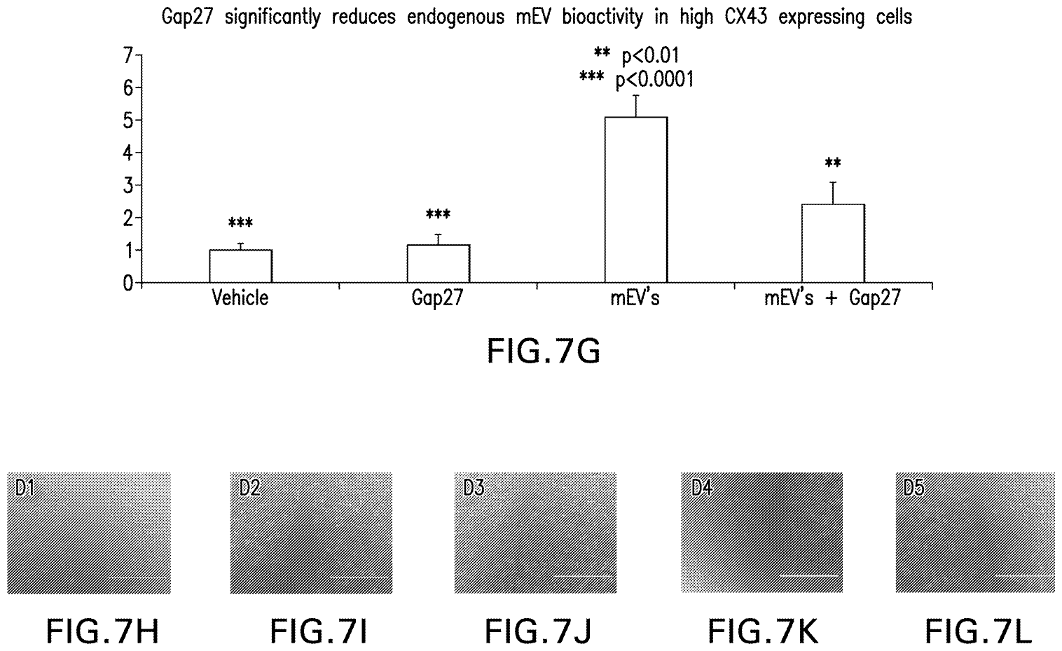

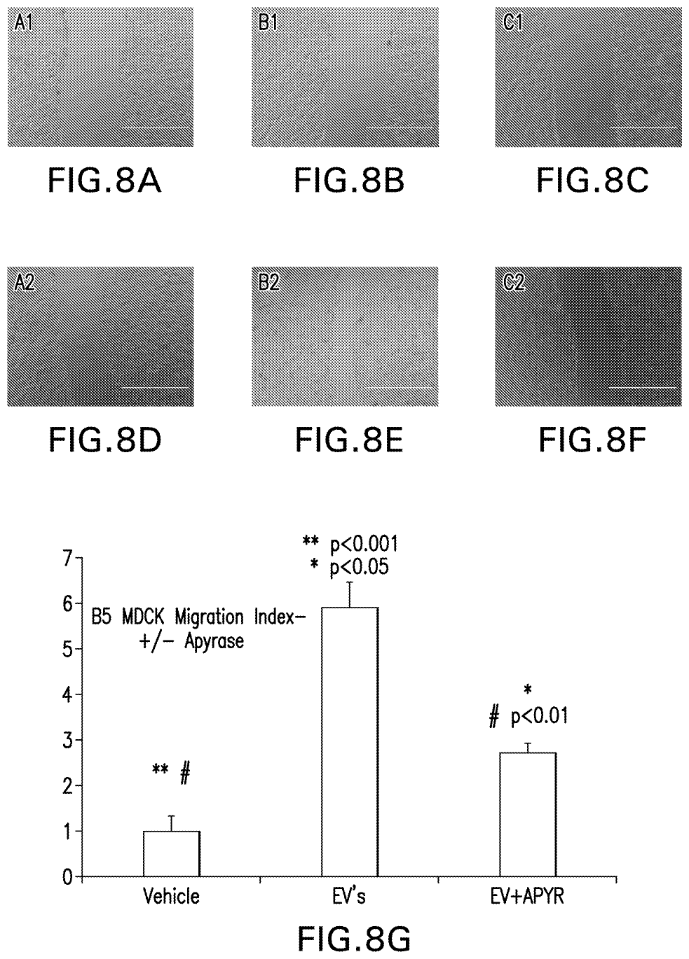

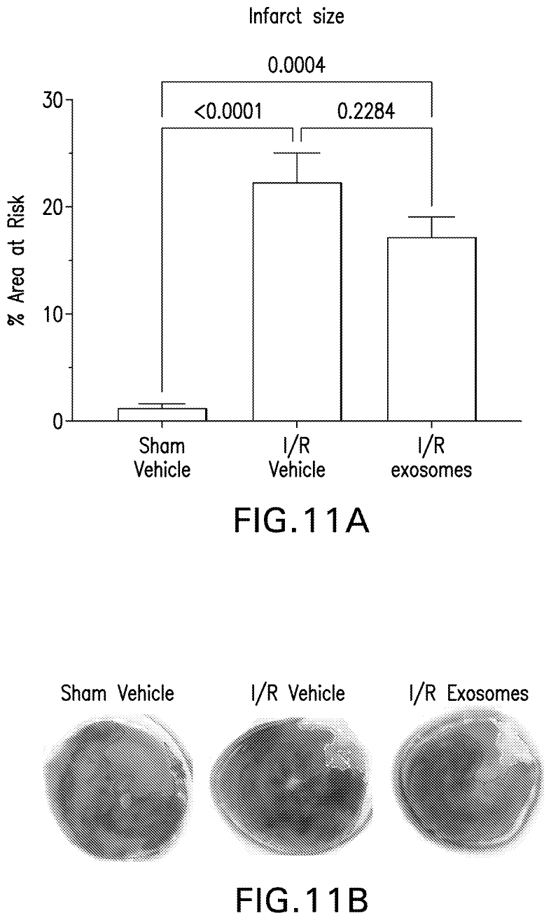

Beyond targeting potential, mEVs also contain highly potent wound healing characteristics. When mEV's are applied to huDFs in a standard scratch wound model and cellular migration is measured (FIG. 6A-6G), we note a wound healing capacity of nearly 50 uM alpha-CT1 (FIG. 6G), a phase III clinical trial wound healing peptide, suggesting that mEV's are equivalent to a 50% dose of a market tested wound healing drug. Migration is measured by measuring scratch wound area at t=0 (FIGS. 6A, 6C, and 6E) and measuring the variation with the final scratch wound area (FIGS. 6B, 6D, and 6F). mEV's statistically increase wound healing capabilities of huDF's in vitro. To then measure the effects of cellular Cx43 expression on migration capacity in mEV treated cell cultures, we duplicated the scratch wound for huDFs in a MDCK model, using Parent Lampe and B5 Clone cell lines (FIG. 7A-7L). Wound healing experiments in MDCK cells mirrored the findings of our mEV targeting experiment, with Parent Lampe cells showing enhanced migration capabilities (2-3×) when provided with mEV's (FIG. 7D) over our vehicle control (FIG. 7C), but with B5 clone cells treated with mEV's (FIG. 7F) showing a 10× enhancement in wound healing capacity over vehicle-treated B5s (FIG. 7E), once again suggesting a critical role of cellular Cx43 in mEV bioactivity—either in wound targeting, or in wound healing capabilities. Applicant then investigated the role of Gap27 blockage of Cx43 in wound healing of mEV's (FIG. 7G), and noted a more than 50% reduction in wound healing capabilities of mEV-treated B5 Clone cells when co-incubated with 100 uM Gap27 (FIG. 7L). Applicant then extended the investigation to that of the role of Apyrase in wound healing abilities of mEV's (FIG. 8A-8G). As with the use of Gap27, the addition of 100 uM Apyrase in tandem with mEV's resulted in a more than 50% reduction in wound healing abilities of mEV's, with data shown in FIG. 8G. This confirms that ATP and Cx43 not only play a key role in the wound-targeting capabilities of mEV's, but that both also play a key role in the downstream effects of mEV's on wounded tissues and cells. To extend this research in vivo, Applicant then orally administered mEV's to mice that were given an LAD-induced cardiac event, then given ischemia-reperfusion to rescue the animal (FIG. 11A-11B). While mEV's did not statistically reduce the scar area in the cardiac tissue, there was a trending 25% reduction in the scar size, which confirms the ability of mEV's to target injured tissues and provide therapeutic benefit in cardiac tissue. Echocardiograms for scar size are shown below the bar graph for each group indicated.

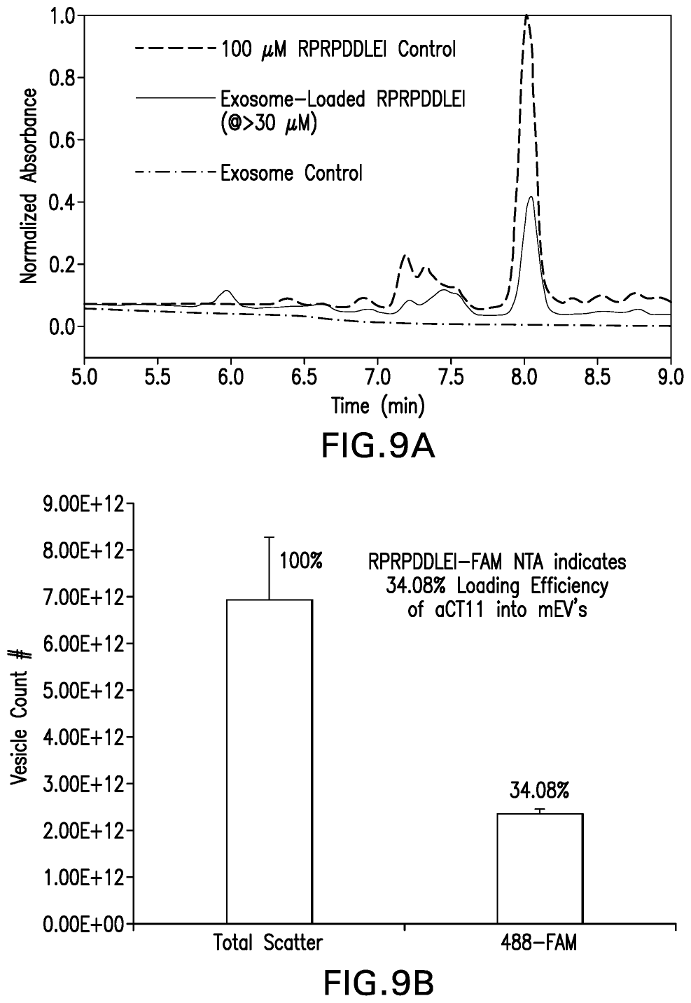

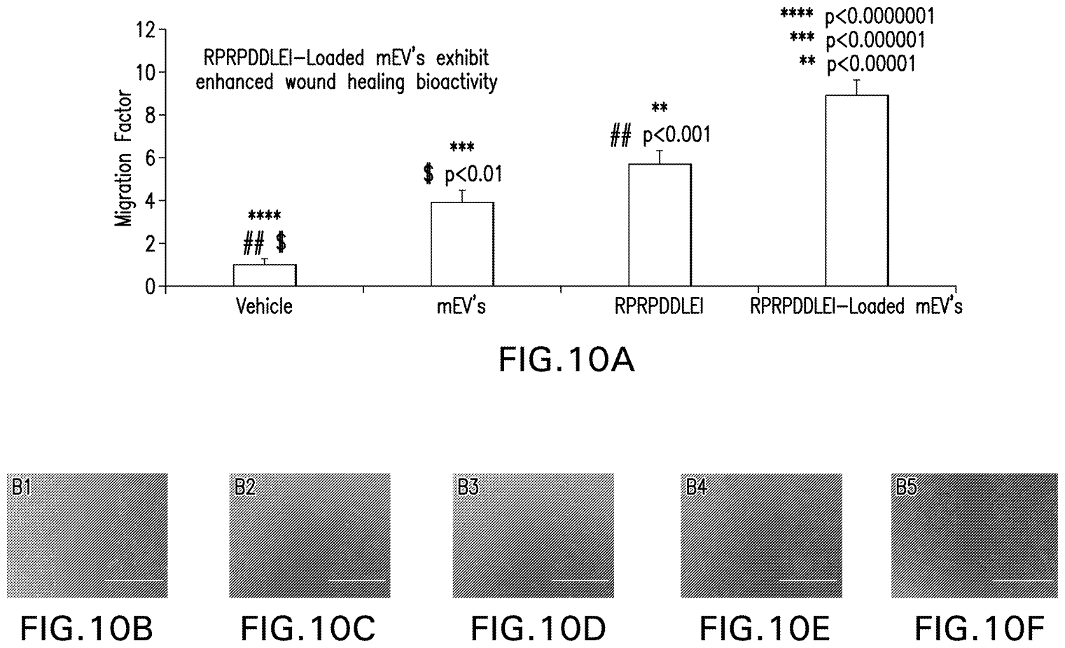

While mEV's show the potential for potent therapeutic applications, we desire to load our biocompatible, non-immunogenic vesicles with exogenous drugs to facilitate a novel drug delivery platform. This was accomplished with slight esterification modifications to our peptide as discussed in a prior disclosure. FIG. 9A-9B shows the HPLC readout (FIG. 9A), with ˜30 uM RPRPDDLEI (SEQ ID NO: 1) loaded into Applicant's mEV's. This was then confirmed via Nanoparticle Tracking Analysis (NTA) for fluorescently tagged mEV's using FAM-conjugation to RPRPDDLEI (SEQ ID NO: 1). NTA analysis confirmed ˜30 uM RPRPDDLEI (SEQ ID NO: 1) loading, with a more precise figure shown in FIG. 9B—˜34% efficiency of loading RPRPDDLEI (SEQ ID NO: 1) into Applicant's mEVs, supporting their use as a cargo (e.g., a drug or there molecule or agent) delivery platform. These RPRPDDLEI-loaded mEV's (SEQ ID NO: 1) were then tested in multiple in vitro models based upon known therapeutic effects of Applicant's peptide via current trials on Alpha-CT1, the parent peptide to RPRPDDLEI (SEQ ID NO: 1). Alpha-CT1 is currently in Clinical trials for dermal wound healing and dermal radiation damage treatment, supporting the use of Alpha-CT1 in both internal wound healing applications as well as internal radiation-induced damage applications. FIG. 10A-10F displays the results from a huDF scratch-wound model, as performed in FIG. 6A-6G, however this experiment included RPRPDDLEI (SEQ ID NO: 1) (FIG. 10E) and RPRPDDLEI-Loaded mEV's (SEQ ID NO: 1) (FIG. 10F). Each component is effective on its' own, creating a significant enhancement in wound healing capabilities, however when treated together as a combination therapeutic post-loading, the effects were compounded (FIG. 10F), resulting in a highly significant enhancement of wound healing capabilities.

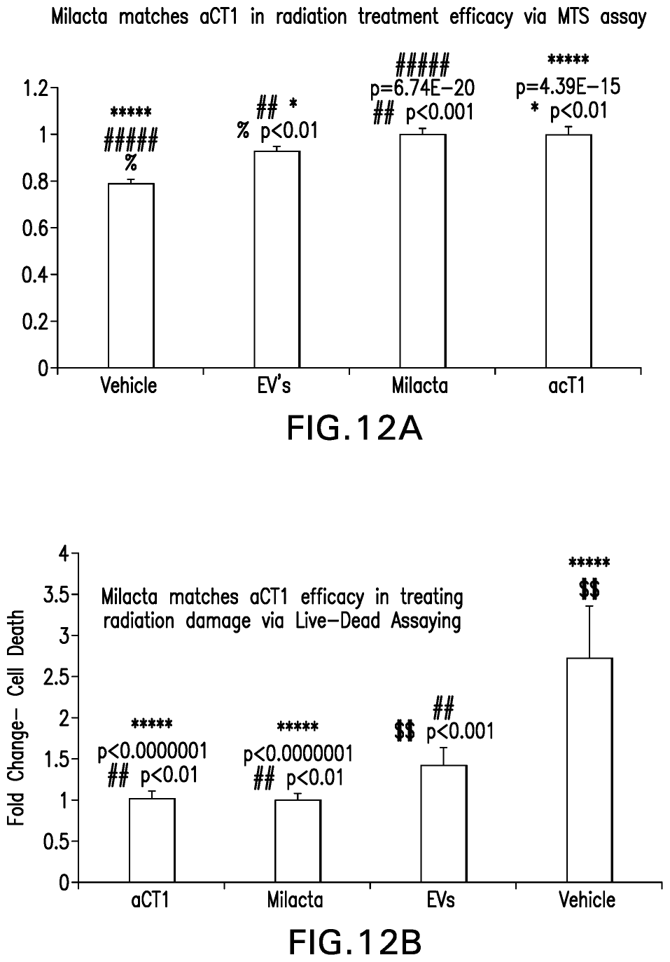

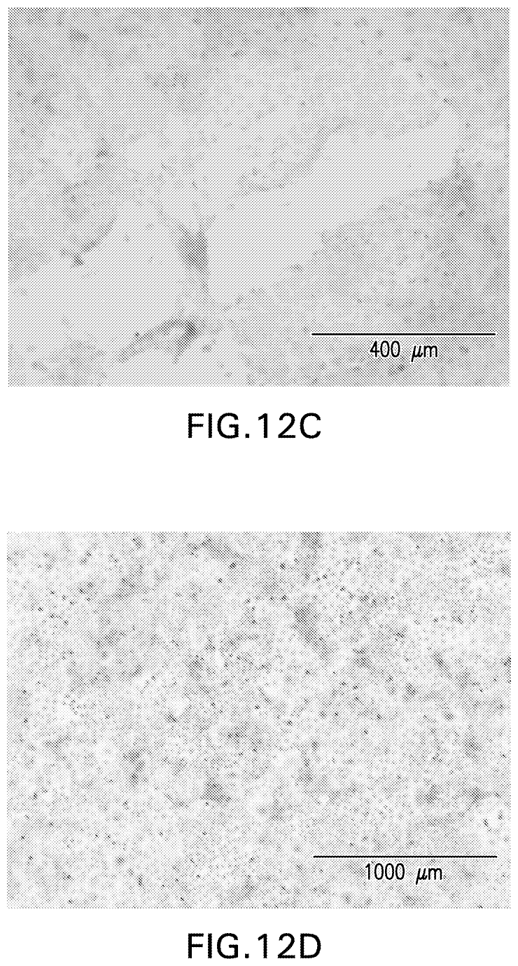

Due to the known therapeutic effects of Alpha-CT1 on dermal radiation damage and without being bound by thereof, Applicant also postulated that the combination therapeutic, RPRPDDLEI-loaded mEV's (SEQ ID NO: 1), would be highly effective at mitigating radiation-induced damage to internal organs. As a test of this, FIG. 12A-12D shows the results from a radiation study using a standard cell model, Intestinal Epithelial Cells (IEC-6). IEC-6 cells were given a 6 Gy dose of radiation from a linear accelerator, then either treated with RPRPDDLEI-loaded mEV's (SEQ ID NO: 1), known clinical therapeutic Alpha-CT1, mEV's or vehicle control. Two assays were used to test the potential radioprotection of Applicant's mEV's and drug delivery system—the Mitochondrial Tracker Assay (MTS) and a Live-Dead Assay (LD). MTS results indicated a highly significant result, with our combination therapeutic besting Alpha-CT1, providing greater radiation protection (FIG. 12A) to a highly significant degree (p<0.1E-20). LD results confirm these findings, with Applicant's therapeutic platform once again slightly edging Alpha-CT1 (FIG. 12B). As shown in FIG. 12A-12B, live-dead images of Applicant's cultures revealed an important fact—vehicle treated samples were experiencing uncontrolled cell death, emanating from a central foci (FIG. 12C). This method of cell death is known as radiation-induced, bystander effect-mediated cell death, in which initial cell death causes a cascade of pro-death signals, then causing additional death. Our treatment platform completely prevents these signals from spreading, eliminating the bystander effect and limiting cell death to only those impacted by the initial radiation insult. These findings confirm that our RPRPDDLEI-loaded mEV's (SEQ ID NO: 1) are a clinical grade radiotherapeutic, providing targeting to injured tissues and potent therapeutic benefits once taken up by resident cells and tissues. RPRPDDLEI-loaded mEV's (SEQ ID NO: 1) target injured tissues, enhance wound healing, and protect against cell death in a number of models—in vitro huDF, MDCK, IEC-6 and in vivo mouse, as well as in a number of wound environments—whether dermal injury, LAD-induced cardiac injury, simple scratch-wounding or even radiation-induced injury, confirming the potency of our therapeutic.

### Example 2

Small extracellular vesicles (EVs) are derived from the phospholipid bilayer of almost every cell in the body and are present in high quantities in milk. Bovine milk exosomes can be loaded with therapeutics and can be a vehicle for oral drug delivery. Exosomes are absorbed systemically, they cross barriers, and have been demonstrated to be absorbed intact. In 2018, the International Society for Extracellular Vesicles (ISEV) released nomenclature guidelines for EVs (Thery et al., 2018). Notably, since consensus on specific markers for EV subtypes, such as exosomes, has not yet been delineated, they recommend that EVs be referred to based on their size, density, composition, or cell of origin (Thery et al., 2018). Throughout this specification, Applicant refers to the EVs by their size (<200 nm [small], or >200 nm [large and/or medium]) (See also, Thery et al., 2018 J Extracell Vesicles. 2018 Nov. 23; 7(1):1535750. doi: 10.1080/20013078.2018.1535750). sEVs are attractive for use as a drug delivery vehicle, particularly for fragile drugs such as insulin or heparin or chemotherapeutics that generally need to be administered via injection. Although efforts to address this need are underway, these efforts have not translated into clinically relevant delivery vehicles or formulations for one reason or another. In many cases insufficient amounts of the EVs are produced for such an approach to be viable. Additionally, many efforts are plagued by stimulating inappropriate immune responses in subjects to which they are administered.

Applicant has focused on using bovine milk sEVs, which as Applicant has determined, can be produced at scale and are generally well tolerated by human subjects. However, it is unknown if these exosomes can be effectively taken up by cells, such as intestinal cells so as to be an effective drug delivery, particularly oral delivery, vehicle. Betker et al., 2019. Demonstrated that an interaction between the neonatal Fc receptor (FcRN) (also referred to as “Fc Receptor” herein) and IgG on the exosome. However, Betker et al., observed a decrease in uptake. The FcRN is expressed throughout the intestinal tract and in many other organs. It is a misnomer, as in humans it is expressed continuously throughout adult life. The receptor binds the Fc portion of IgG and functions as a means to transport the IgG across a cell. IgG that is bound to the Fc receptor has been shown to avoid lysosomal degradation. The Fc receptor functions only at an acidic pH. The prevailing understanding is that the majority of IgG binding to an FcRN actually occurs when IgG has already entered the cell. Here, IgG is contained in acidified endosomes with FcRN present. The FcRN binds its target in the acidified environment.

The neonatal FC receptor can modulate oral bioavailability of bovine milk exosome drug delivery. The neonatal Fc receptor (FcRN) on gut epithelium can bind to IgG on bovine milk exosomes and can mediate uptake. Without being bound by theory, once bound to the Fc receptor, IgG is transported to the basolateral side of polar cells (like enterocytes of the gut epithelium or of the blood-brain barrier). It behaves in a bidirectional manner to transport IgG from the apical to the basolateral membrane, or vice versa, and recycles back to the membrane. In other words, it mediates transcytosis within the cells. Targeting therapeutics or delivery vehicles to the Fc receptor can be a way to increase the uptake and/or bioavailability of a therapeutic or other agent. It may also provide protection against degradation as the sEVs pass through the cell. See also, Pridgen et al., 2013; Hornby et al., 2013; Dickinson et al., 1999; McCarthy et al., 2000; Yoshida et al., 2004; Roopenian and Akilesh, 2007; and Agrawal et al., 2017).

This Example at least demonstrates targeting the Fc receptor via IgG for improved uptake and/or transfer of the exosomes into and transport through cells. Without being bound by theory, this can allow the exosome and/or its cargo to avoid lysosomal degradation within the cell. This can lead to improved bioavailability, such as oral bioavailability, of the cargo, including but not limited to therapeutic cargo.

Applicant further investigated the mechanism of milk exosome uptake by intestinal cells, particularly intestinal epithelial cells. Without being bound by theory, Applicant hypothesized that bovine milk exosomes are taken up by intestinal epithelial cells via an interaction between exosome bound IgG and an Fc receptor, such as the neonatal Fc receptor. Applicant determined that IgG is present on small EVs.

Exosomes were produced using a methodology described in e.g., the method set forth in Pridgen, Eric M et al. “Transepithelial transport of Fc-targeted nanoparticles by the neonatal fc receptor for oral delivery.” Science translational medicine vol. 5,213 (2013): 213ra167 or WO 2022/182782, which are incorporated by reference as if expressed in their entireties herein. Briefly, Unpasteurized, full fat, fresh bovine milk is defatted by undergoing a series of centrifugation steps. It was then filtered using by Millipore 0.45 m and 0.22 m filters. Calcium was chelated with 30 mM EDTA at about 37° C. for 60 minutes with gentle stirring. Then, it was filtered using a Repligen KrosFlo TFF system into HEPES buffer. Finally, size exclusion chromatography is done using a IZON qEV original 70 nm sepharose column and fractioned into a 96 well plate. Exosome fractions 8.0-9.0 yielded high quantities of pure exosomes seen by Negative stain electron microscopy of final isolates, showing ultra-dense accumulation of sEVs in peak SEC fractions, and Nanoparticle Tracking Analysis (NTA) Protein concentration of sEV containing solution following SEC from three separate isolations. Fractions with peak protein concentration are fractions 8.0-9.0. Reproducibility across multiple replicates was observed.

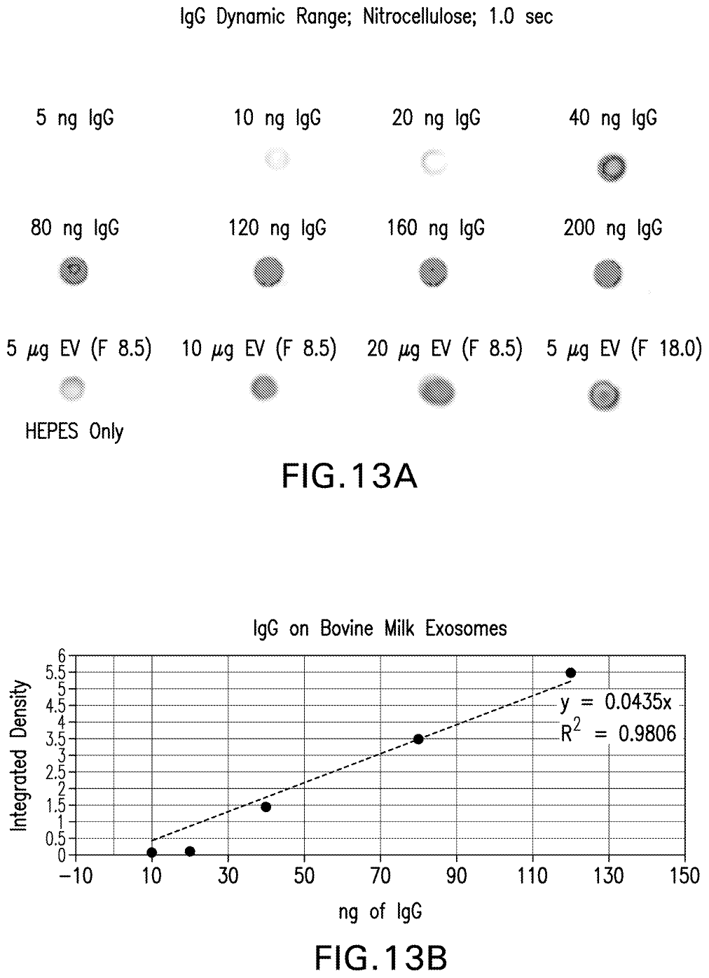

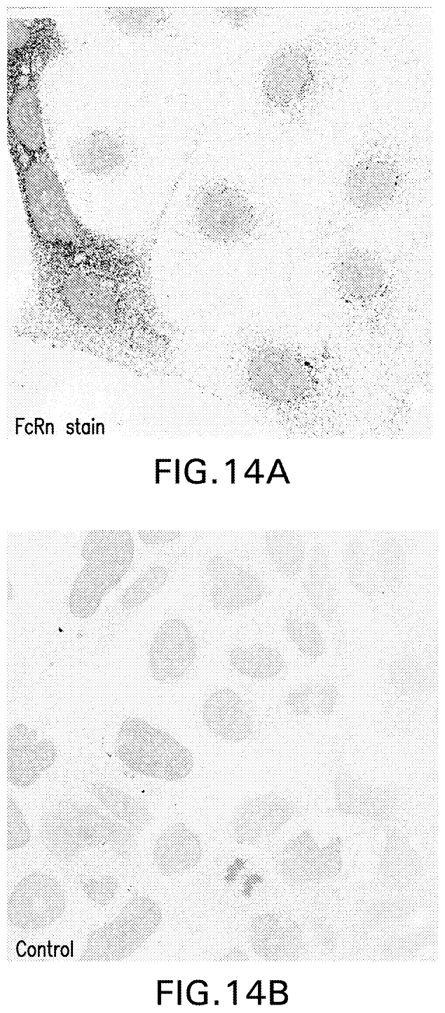

Applicant then evaluated the bovine sEVs for the presence of IgG. Bovine milk exosomes were isolated as before. A dot blot on nitrocellulose was performed using an IgG standard curve with IgG from bovine serum albumen. The working range of the standard curve ranged from 5 ng IgG to 200 ng IgG. 5 μg, 10 μg, and 20 μg, of isolated bovine milk exosomes were analyzed. Also analyzed was 5 μg of F 18 (where casein is seen). Control was HEPES only. The blot was incubated in rabbit anti-bovine IgG HRP for about one hour. The blot was blocked in fish skin (3%) and imaged using chemilumiescent reagents via chemidoc). Analysis of the image showed there was about 1.42 μg of IgG per μg of exosome. FIG. 13A-13B demonstrates that IgG is present on small EVs. (FIG. 13A) IgG Dynamic range as tested on nitrocellulose (1.0 second). (FIG. 13 B) Demonstrates IgG presence on bovine Milk Exosomes.

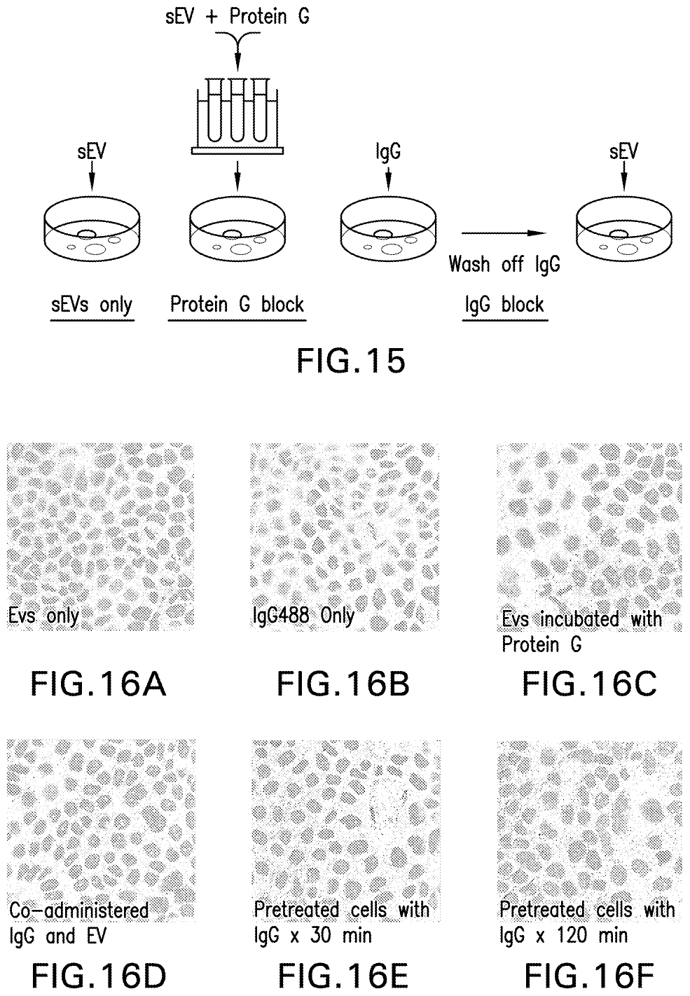

Applicant also evaluated if FcRN is present on Caco2 cells. Caco-2 cells were used as they are a standard model in vitro cell line for intestinal uptake, including pharmaceutical uptake, evaluation. Briefly cells were plated at a density of about 0.2×106 cells/plate and grown to confluence (N−3). Cells were fixed with paraformaldehyde, rinsed with phosphate buffered saine with Tris (PBST) solution and blocked with bovine serum album (BSA). Then cells were incubated in a rabbit anti-FcRn antibody overnight. They were washed again and then incubated with an appropriate secondary antibody (e.g., donkey anti-rabbit IgG) for about 1 hour then washed again. Nuclei were stained with Hoecht stain. Control cells were stained with secondary antibody only. Cells were imaged with confocal microscopy and analyzed with Image J. See e.g., FIG. 14A-14B. Preliminary testing showed that small EVs are taken up into Caco-2 cells in high quantities within 30 minutes of incubation time. Preliminary testing demonstrated that pH did not affect uptake. The experimental procedure outlined in FIG. 15 provides further evaluation by blocking interaction between FcRN and IgG that is present on the surface of a small EV (sEV). Protein G was used to bind and inhibit IgG. Free IgG was used to bind FcRN. Briefly cells were loaded with CellTracker™ Deep Red dye (Thermo Fischer Scientific) according to the manufacturer's instructions. Excess, unloaded dye was removed by centrifuging in a SpinOUT™ (G-Biosciences) 3 ml column at 16,873 xg for 60 minutes at 4° C. The sEV uptake experiments were conducted with 10 treatment groups (Table 1), each consisting of 3 replicates per group. For experiments requiring protein G, recombinant protein G (Pierce) was added in 5× the molar concentration of IgG (as calculated by dot blot in FIG. 13A). Protein G was chosen because it has a higher affinity to bovine IgG than protein A. The protein G-sEV solution was allowed to incubate for 60 minutes at RT. Subsequently, the unbound protein G was removed using a Micron YM100 column (Sigma Aldrich) per manufacturer's instructions. For treatment groups 1-6, IgG from bovine serum (Sigma Aldrich) or AlexaFluor 488 ChromPure bovine IgG (JacksonImmuno, whole molecule) (488-IgG) was added to 0.5 mL of Hank's Balanced Salt Solution (HBSS; pH 7.4) in 50× the molar concentration of sEV IgG and incubated on the Caco-2 cells for the indicated amount of time. For all groups except group 10, cells were washed twice with HBSS solution before incubating with 25 μg of CTDR-sEVs or unlabeled-EVs in 0.5 mL HBSS at 37° C. for 30 minutes. Subsequently, cells were washed twice with DBSS, fixed in 2% paraformaldehyde for 10 minutes, and washed three times in PBS. DNA was visualized with 1:30,000 Hoechst 3342 Solution (Thermo Fisher Scientific).

TABLE 1
Treatment Groups

| Group | Treatment |
|---|---|
| 1 | CTDR-sEVs were administered at the same time as IgG from |
| | bovine serum (Sigma Aldrich) |
| 2 | CTDR-sEVs were administered at the same time as AlexaFlour |
| | 488 ChromPure bovine IgG (Jacksonimmuno, whole molecule) |
| 3 | Caco-2 cells were treated with IgG from bovine serum |
| | (Sigma Aldrich) for 30 minutes before CTDR-sEVs added |
| 4 | Caco-2 cells were treated with AlexaFlour 488 ChromPure |
| | bovine IgG (Jacksonimmuno, whole molecule) 30 minutes |
| | before CTDR-sEVs added |
| 5 | Caco-2 cells were treated with IgG from bovine serum |
| | (Sigma Aldrich) for 120 minutes before CTDR-sEVs added |
| 6 | Caco-2 cells were treated with AlexaFlour 488 ChromPure |
| | bovine IgG (Jacksonimmuno, whole molecule) 120 minutes |
| | before CTDR-sEVs added |
| 7 | CTDR-sEVs were incubated with Protein G (Pierce, VH321503) |
| | prior to being applied to Caco-2 cells |
| 8 | CTDR-sEVs were applied to Caco-2 cells with no “block” |
| 9 | Unlabeled-sEVs were applied to Caco-2 cells with no “block” |
| 10 | AlexaFlour 488 chromPure bovine IgG (Jacksonimmuno, |
| | whole molecule) was applied to Caco-2 cells without sEVs |

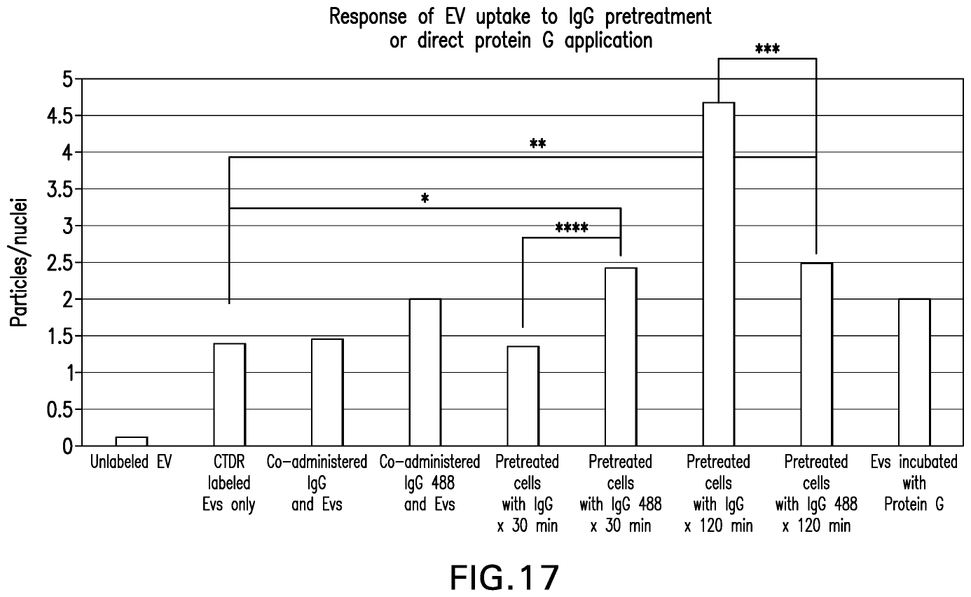

FIG. 16A-16F shows results of uptake of various treatment groups in Caco-2 cells. FIG. 17 shows the response of EV uptake to IgG pretreatment or direct protein G application. * denotes statistical significance (P<0.05). The uptake of CTDR-sEVs into Caco-2 cells was quantified by counting the number of particles per nuclei present in laser scanning confocal microscope images using ImageJ. The metric “particles per nuclei” was chosen in order to ensure that the average sEV uptake per cell was being quantified. 488-IgG was used in this experiment to assess the functionality of FcRN. FIG. 3 demonstrates that 488-IgG is being taken up into Caco-2 cells and thus the FcRn-IgG interaction able to occur in this assay. FIG. 16A-16F also shows that there are high amounts of intercell variability in terms of CTDR-sEV uptake. For example, in FIG. 16D there was more sEV uptake in the cells in the center of the image when compared to the cells in the bottom left of the image. This relationship was also observed when looking at the 488-IgG uptake. There is not a significant decrease in the quantity of CTDR-sEVs taken up into Caco-2 cells when a block is introduced (FIG. 16A-16F). No significant difference was observed between the uptake of CTDR-sEVs with no block and CTDR-sEVs blocked with protein G or between CTDR-sEVs with no block and CTDR-sEVs that were co-administered with IgG.

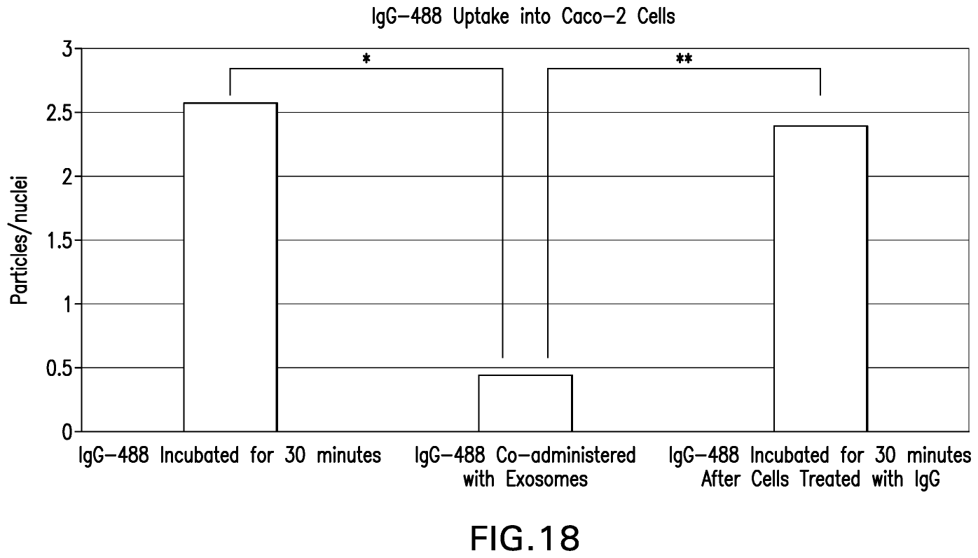

FIG. 18 shows IgG-488 uptake into Caco-2 cells. FIG. 18 shows the particles of 488-IgG per nuclei. * denotes statistical significance (P<0.05). 488-IgG was used as a means to prove the functionality of the FcRn but also to compare uptake to that of sEVs. FIG. 5 compares the uptake of 488-IgG after being incubated on Caco-2 cells for 30 minutes when given alone (no CTDR-sEVs), when co-administered with CTDR-sEVs, and when given as a pretreatment before CTDR-sEVs. 488-IgG uptake is significantly (P<0.05) less when incubated simultaneously with CTDR-sEVs when compared to when administered prior to sEVs. When 488-IgG is allowed to incubate on Caco-2 cells without sEVs, there is no difference in CTDR-sEV uptake (FIG. 5). The IgG was administered in a concentration of 50 times that of sEV-IgG, so this result was not expected. Notably, co-administering 488-IgG with CTDR-sEVs does not affect the uptake of CTDR-sEVs when compared to the control (FIG. 17).

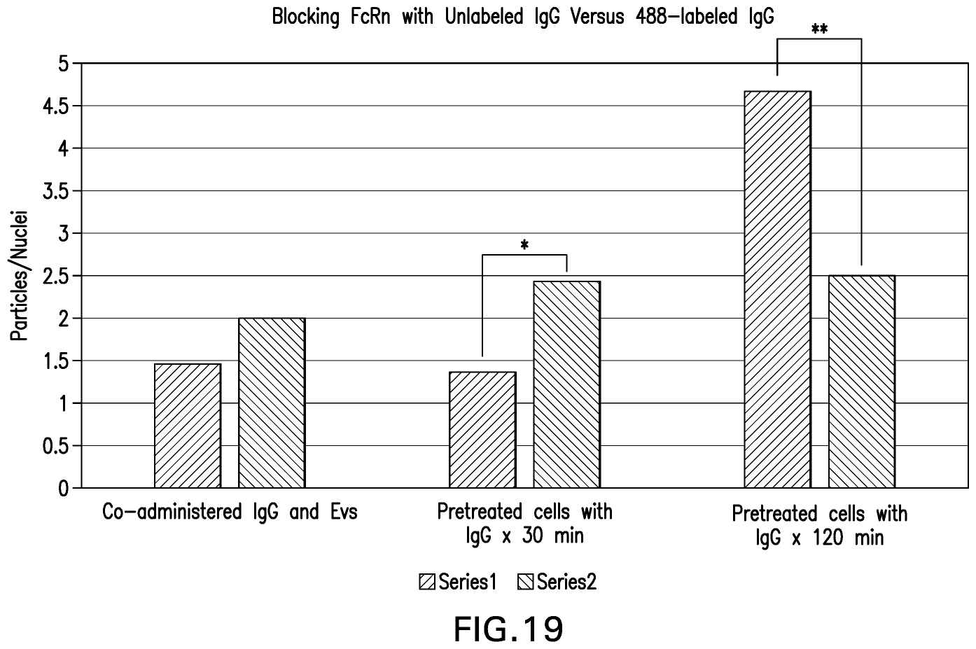

FIG. 19 shows results from blocking FcRN with unlabeled IgG as compared to 488-labeled IgG. FIG. 19 demonstrates the particles of CTDR-sEV per nuclei of each group in which FcRn was “blocked”. Dark gray bars represent cells treated with unlabeled-IgG. Light gray bars represent cells treated with 488-IgG. * denotes statical significance (P<0.05). Interestingly, in groups where cells were pretreated with 488-IgG for 30 minutes or 120 minutes, there is a significant (P<0.05) increase in the amount of CTDR-sEVs internalized when compared to the control (FIG. 17). Notably, FIG. 4 also demonstrates a significant intra-group difference when comparing cells pretreated with unlabeled-IgG versus 488-IgG (P<0.05). The difference between these groups is not consistent in that cells pretreated with 488-IgG for 30 minutes have a greater CTDR-sEV uptake than cells pretreated with unlabeled-EV for 30 minutes, but cells pretreated with 488-IgG for 120 minutes have significantly (P<0.05) less CTDR-sEV uptake than cells pretreated with unlabeled-EVs for 120 minutes. The difference in molar mass between the unlabeled-IgG and the 488-IgG was accounted for in the calculations.

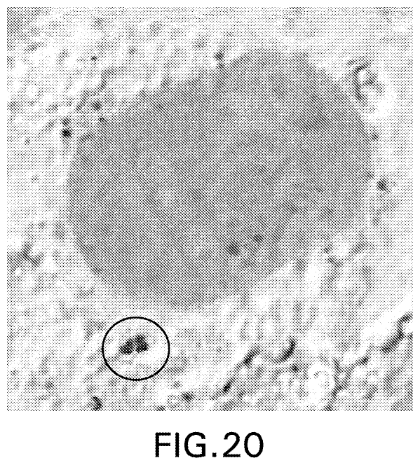

FIG. 20 shows IgG and EV colocalization (within circled region in FIG. 20). FIG. 20 shows a single Caco-2 cell with CTDR-sEVs visualized by red fluorescence, 488-IgG visualized with green fluorescence (as represented in greyscale), and nuclei visualized with blue fluorescence (as represented in greyscale). There is apparent grouping of the CTDR-sEV and 488-IgG in the upper left and lower left areas of the cell.

In contrast to Betker et al., 2019 in vivo, Applicant determined that blocking the interaction of sEV-IgG and FcRN did not decrease sEV uptake in Caco-2 cells. Without being bound by theory, there may be more than one mechanism that are responsible for sEV internalization by cells. These results also suggest an interaction between sEVs and FcRN in an intestinal model. Further, these results suggest that pretreating intestinal cells with IgG can increase sEV uptake. Without being bound by theory, these results suggest that pretreating, co-administering, or incorporating IgG with the sEVs can be viable approaches for increasing uptake of sEVs, such as bovine EVs loaded with cargo in the intestine. This can lead to increased oral bioavailability of the cargo.

### References for Example 2

Betker, Jamie L., et al. “The Potential of Exosomes From Cow Milk for Oral Delivery.” Journal of Pharmaceutical Sciences, vol. 108, no. 4, 2019, pp. 1496-1505.
Manca, Sonia et al. “Milk exosomes are bioavailable and distinct microRNA cargos have unique tissue distribution patterns.” Scientific reports vol. 8,1 11321. 27 Jul. 2018
Munagala, Radha et al. “Bovine milk-derived exosomes for drug delivery.” Cancer letters vol. 371,1 (2016): 48-61. doi:10.1016/j.canlet.2015.10.020
Pridgen, Eric M et al. “Transepithelial transport of Fc-targeted nanoparticles by the neonatal fc receptor for oral delivery.” Science translational medicine vol. 5,213 (2013): 213ra167.
Sockolosky, Jonathan T, and Francis C Szoka. “The neonatal Fc receptor, FcRn, as a target for drug delivery and therapy.” Advanced drug delivery reviews vol. 91 (2015): 109-24.
Dickinson, B L et al. “Bidirectional FcRn-dependent IgG transport in a polarized human intestinal epithelial cell line.” The Journal of clinical investigation vol. 104,7 (1999): 903-11.
Pyzik, Michal et al. “The Neonatal Fc Receptor (FcRn): A Misnomer?.” Frontiers in immunology vol. 10 1540. 10 Jul. 2019
Pridgen, Eric M et al. “Transepithelial transport of Fc-targeted nanoparticles by the neonatal fc receptor for oral delivery.” Science translational medicine vol. 5,213 (2013): 213ra167.
Agrawal et al., Nanomedicine 2017 July; 13(5):1627-1636. doi: 10.1016/j.nano.2017.03.001. Epub 2017 Mar. 11.
Adriano et al., Bioactive Materials. 6(8): 2479-2490 (2021)
McCarthy et al., 2000. J. Cell Sci., 113 (2000), pp. 1277-1285.
Yoshida et al., 2004. Immunity. 20(6): 769-783
Hornby et al., Pharm Res. 2013 doi: 10.1007/s11095-013-1212-3
Roopenian D C, Akilesh S. FcRn: the neonatal Fc receptor comes of age. Nat Rev Immunol. 2007; 7(9):715-725

### Example 3—Connexin43 Expression and Function in Bovine Milk-Derived Exosomes

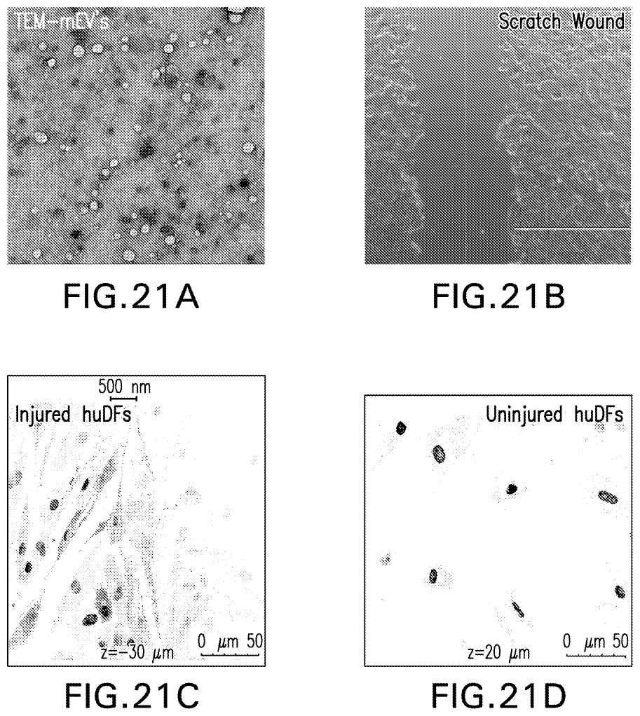

Small extracellular vesicles known as exosomes (nanovesicles of less than or equal to 150 nm diameter) are secreted by nearly all cell types and are thought to have key assignments in intercellular signaling. Reports that exosomes evade immune detection, show propensity to cross tissue boundaries (e.g., the blood-brain barrier) and can be orally administered has also attracted increasing interest in therapeutic applications, including as drug delivery devices. Applicant recently developed a method for large-scale purification of bovine milk-derived extracellular vesicles (Marsh et al., Nanotheranostics. 2021 Jul. 5; 5(4):488-498. doi: 10.7150/ntno.62213. eCollection 2021). From 1000 mLs of milk, this method yields over 150 mL of purified extracellular vesicles (mEVs) at ultra-dense concentration (>1×10¹² mEVs/mL, FIG. 21A). Girao and co-workers determined that Connexin 43 (Cx43) is present in exosomes derived from cultured cells expressing Cx43, where this gap junction protein has assignments in cellular uptake of exosomal cargo (Soares et al., Sci Rep. 2015 Aug. 19; 5:13243. doi: 10.1038/srep13243). Applicant used Western blotting with multiple antibodies against different Cx43 epitopes, determining that a carboxyl-terminal truncated Cx43 isoform is likely present in bovine mEVs. Immuno-electron microscopy also suggested that a significant proportion of mEVs demonstrated signals consistent with Cx43. To probe the role of Cx43 in milk-derived exosomes, we undertook studies of response to mEVs in scratch-wounded monolayers (FIG. 21B) of Human Dermal Fibroblasts and Madin-Darby Canine Kidney (MDCK) lines expressing high (MDCK Cx43-high) and low levels of Cx43 (MDCK Cx43-low). These results indicate that scratch-wounding (FIG. 21C) in all three cell lines resulted in significant (p<0.05) increases in mEV uptake over uninjured cells (FIG. 21D). Interestingly, baseline levels of mEV uptake in uninjured MDCK Cx43-high cells were an order magnitude higher than that of MDCK Cx43-low cells—a difference that was further increased by scratch wounding. In ongoing work, we seek to further probe the function of mEV Cx43 using in vitro models of cellular injury, as well as determine whether mEV uptake is also upregulated following injury in vivo.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:
1. A milk exosome comprising: an exogenous cargo, wherein the milk exosome is capable of targeting an injury or site thereof or a cancer cell or cancer cell population by targeting ATP secretion and/or concentration and/or neonatal Fc receptor (FcRN).
2. The milk exosome of aspect 1, wherein the injury is a mechanical injury or a non-mechanical injury.
3. The milk exosome of aspect 2, wherein the non-mechanical injury is a chemical injury, electrical injury, radiation injury, an inflammatory injury, or an ischemic injury.
4. The milk exosome of aspect 2, wherein the mechanical injury is a wound or burn.
5. The milk exosome of any one of aspects 1-4, wherein the injury or site thereof has a greater concentration of ATP than a non-injury or site thereof.
6. The milk exosome of any one of aspects 1-5, wherein the cancer cell or cancer cell population has or has a microenvironment that has a greater concentration of ATP than a microenvironment of a non-cancer cell or non-cancer cell population.
7. The milk exosome of any one of aspects 1-6, wherein the cancer cell or cancer cell population secretes ATP.
8. The milk exosome of any one of aspects 1-7, wherein the milk exosome does not contain an exogenous targeting moiety.
9. The milk exosome of any one of aspects 1-8, wherein the milk exosome comprises connexin 43.
10. The milk exosome of aspect 9, wherein the connexin 43 is a native connexin 43 or wherein the connexin 43 is an engineered connexin 43.
11. The milk exosome of any one of aspects 1-10, wherein the exogenous cargo is a biologic molecule.
12. The milk exosome of aspect 11, wherein the biologic molecule is a polypeptide, peptide, or a nucleic acid.
13. The milk exosome of any one of aspects 1-12, wherein the exogenous cargo is an ACT1 peptide, ACT11 peptide, an ACT minus peptide, a selectide, or any combination thereof.
14. The milk exosome of any one of aspects 1-13, wherein the milk exosome is a bovine milk exosome.
15. The milk exosome of any one of aspects 1-15, wherein the milk exosome, prior to containing the exogenous cargo, is isolated according to a method comprising:
   a. centrifuging a mammalian milk under conditions suitable to separate fats from one or more other components of the mammalian milk;
   b. removing the separated fats from the mammalian milk;
   c. after step (b) centrifuging the remaining mammalian milk one or more times and skimming any noticeable separated fats after each centrifuging in step (c);
   d. filtering the remaining biological fluid after step (c)
   e. optionally performing one or more ultracentrifugation steps after (d);
   f. chelating divalent cations with about 10 mM to about 100 mM EDTA at about 30-42 degrees Celsius after (d) or optionally (e) and optionally for about 15-120 minutes; and
   g. after (f), optionally performing tangential flow filtration to obtain a retentate, wherein the retentate is optionally ultracentrifuged via one or more ultracentrifugation steps or stored at −80 degrees C., and separating out fractions of the retentate, optionally via column separation, after the retentate is optionally ultracentrifuged or stored at −80 degrees C., wherein the method comprises step (e) or step (g) but not both.
16. A population of milk exosome comprising one or more milk exosomes as in any one of aspects 1-15.
17. The population of milk exosomes of aspect 16, wherein the population is enriched for connexin 43 positive milk exosomes.
18. The population of milk exosomes of aspect 16, wherein the milk exosome, prior to containing the exogenous cargo, is isolated according to a method comprising
   a. centrifuging a mammalian milk under conditions suitable to separate fats from one or more other components of the mammalian milk;
   b. removing the separated fats from the mammalian milk;
   c. after step (b) centrifuging the remaining mammalian milk one or more times and skimming any noticeable separated fats after each centrifuging in step (c);
   d. filtering the remaining biological fluid after step (c)
   e. optionally performing one or more ultracentrifugation steps after (d);
   f. chelating divalent cations with about 10 mM to about 100 mM EDTA at about 30-42 degrees Celsius after (d) or optionally (e) and optionally for about 15-120 minutes; and
   g. after (f), optionally performing tangential flow filtration to obtain a retentate, wherein the retentate is optionally ultracentrifuged via one or more ultracentrifugation steps or stored at −80 degrees C., and separating out fractions of the retentate, optionally via column separation, after the retentate is optionally ultracentrifuged or stored at −80 degrees C.,
      wherein the method comprises step (e) or step (g) but not both.
      and further comprising enriching the milk exosomes for connexin 43 expressing milk exosomes.
19. The population of milk exosomes of aspect 18, further comprising enriching the milk exosomes for connexin 43 expressing milk exosomes.
20. The population of milk exosomes of any one of aspects 16-19, wherein the milk exosomes do not contain an exogenous targeting moiety.
21. The population of milk exosomes of any one of aspects 16-20, wherein the exogenous cargo is a biologic molecule.
22. The population of milk exosomes of aspect 21, wherein the biologic molecule is a polypeptide, peptide, or a nucleic acid.
23. The population of milk exosomes of any one of aspects 16-22, wherein the exogenous cargo is an ACT1 peptide, ACT11 peptide, an ACT minus peptide, a selectide, or any combination thereof.
24. A pharmaceutical formulation comprising:
   a milk exosome as in any one of aspects claim 1-15 or a population thereof; and
   a pharmaceutically acceptable carrier.
25. The pharmaceutical formulation of aspect 24, further comprising an agent capable of stimulating ATP release, secretion, and/or production, ATP, ADP, or any combination thereof.
26. The pharmaceutical formulation of aspect 25, wherein the pharmaceutical formulation is in a dosage form that releases the agent capable of stimulating ATP release, secretion, and/or production, ATP, ADP, or any combination thereof prior to the milk exosome or population thereof thereby delivering the agent capable of stimulating ATP release, secretion, and/or production, ATP, ADP, or any combination thereof prior to the milk exosome.
27. The pharmaceutical formulation of any one of aspects 24-26, further comprising one or more immunoglobulins.
28. The pharmaceutical formulation of aspect 25, wherein the one or more immunoglobulins are selected from IgG, IgM, IgA, IgD, and/or IgE.
29. The pharmaceutical formulation of any one of aspects 27-28, wherein the pharmaceutical formulation is in a dosage form that releases the immunoglobulin prior to the milk exosome or population thereof thereby delivering the immunoglobulin prior to the milk exosome.
30. A method of treating an injury at an injury site or treating a cancer in a subject in need thereof comprising:
   administering to the subject in need thereof, a milk exosome of claim 1 or a population thereof or a pharmaceutical formulation comprising the milk exosome of claim 1 or the population thereof.
31. The method of aspect 30, wherein the milk exosome is a bovine milk exosome.
32. The method of any one of aspects 30-31, wherein the milk exosome does not contain an exogenous targeting moiety.
33. The method of any one of aspects 30-32, wherein the milk exosome comprises connexin 43.
34. The method of aspect 33, wherein the connexin 43 is a native connexin 43 or wherein the connexin 43 is an engineered connexin 43.
35. The method of any one of aspects 30-34, wherein the exogenous cargo is a biologic molecule.
36. The method of aspect 35, wherein the biologic molecule is a polypeptide, peptide, or a nucleic acid.
37. The method of any one of aspects 30-36, wherein the exogenous cargo is an ACT1 peptide, ACT11 peptide, an ACT minus peptide, a selectide, or any combination thereof.
38. The method of any one of aspects 30-38, further comprising administering an amount of one or more immunoglobulins to the subject in need thereof.
39. The method of aspect 38, wherein administering the amount of one or more immunoglobulins to subject in need thereof occurs prior to administering the amount of a milk exosome of claim 1 or a population thereof or a pharmaceutical formulation comprising the milk exosome of claim 1 or the population thereof.
40. The method of any one of aspects 38-39, wherein the one or more immunoglobulins are selected from are selected from IgG, IgM, IgA, IgD, and/or IgE.
41. The method of any one of aspects 38-40, wherein the one or more immunoglobulins consists of or comprises IgG.
42. The method of any one of aspects 38-41, wherein the one or more immunoglobulins increase uptake of the milk exosome or population thereof in a cell.
43. The method of aspect 42, wherein the cell is a polarized cell.
44. The method of any one of aspects 42-43, wherein the cell is a gastrointestinal cell, a heart cell, a liver cell, brain cell, nerve cell, reproductive cell, cancer cell, or any combination thereof.
45. The method of any one of aspects 30-44, further comprising administering one or more agents that stimulate ATP release, secretion, and/or production, ATP, ADP, or any combination thereof to the subject in need thereof.
46. The method of aspect 45, wherein administering the one or more agents that stimulate ATP release, secretion, and/or production, ATP, ADP, or any combination thereof to subject in need thereof occurs prior to administering the amount of a milk exosome of claim 1 or a population thereof or a pharmaceutical formulation comprising the milk exosome of claim 1 or the population thereof or a pharmaceutical formulation comprising the milk exosome of claim 1.
47. The method of claim any one of aspects 45-46, wherein the one or more agents that stimulate ATP release, secretion, and/or production, ATP, ADP, or any combination thereof increase the cellular uptake of the milk exosome or population thereof in a cell.
48. The method of aspect 47, wherein the cell is a polarized cell.
49. The method of any one of aspects 47-48, wherein the cell is a gastrointestinal cell, heart cell, liver cell, brain cell, nerve cell, reproductive cell, cancer cell, or any combination thereof.
50. The method of any one of aspects 30-49, wherein the injury is a mechanical injury site or a non-mechanical injury.
51. The method of aspect 50, wherein the non-mechanical injury is a chemical injury, electrical injury, radiation injury, or an ischemic injury.
52. The method of aspect 50, wherein the mechanical injury is a wound or burn.
53. The method of any one of aspects 30-52, wherein the milk exosome or population thereof and/or pharmaceutical formulation are irradiated, sterilized, or both.
54. A method of treating a wound or cancer in a subject comprising: administering a milk exosome and an agent capable of stimulating ATP to the subject in need thereof.
55. The method of claim 54, wherein the milk exosome is as in any one of claims 1-15.
56. The milk exosome, population of milk exosomes, or pharmaceutical formulation of any one of claims 1-29, wherein the milk exosome or population thereof and/or pharmaceutical formulation thereof are irradiated, sterilized, or both.

What is claimed is:

1. A milk exosome comprising:
an exogenous cargo, wherein the milk exosome is capable of targeting an injury or site thereof or a cancer cell or cancer cell population by targeting ATP secretion and/or concentration and/or neonatal Fc receptor (FcRN).

2. The milk exosome of claim 1, wherein the injury is a mechanical injury or a non-mechanical injury.

3. The milk exosome of claim 2, wherein the non-mechanical injury is a chemical injury, electrical injury, radiation injury, an inflammatory injury, or an ischemic injury.

4. The milk exosome of claim 2, wherein the mechanical injury is a wound or burn.

5. The milk exosome of claim 1, wherein the injury or site thereof has a greater concentration of ATP than a non-injury or site thereof.

6. The milk exosome of claim 1, wherein the cancer cell or cancer cell population has or has a microenvironment that has a greater concentration of ATP than a microenvironment of a non-cancer cell or non-cancer cell population.

7. The milk exosome of claim 1, wherein the cancer cell or cancer cell population secretes ATP.

8. The milk exosome of claim 1, wherein the milk exosome does not contain an exogenous targeting moiety.

9. The milk exosome of claim 1, wherein the milk exosome comprises connexin 43.

10. The milk exosome of claim 9, wherein the connexin 43 is a native connexin 43 or wherein the connexin 43 is an engineered connexin 43.

11. The milk exosome of claim 1, wherein the exogenous cargo is a biologic molecule.

12. The milk exosome of claim 11, wherein the biologic molecule is a polypeptide, peptide, or a nucleic acid.

13. The milk exosome of claim 1, wherein the exogenous cargo is an ACT1 peptide, ACT11 peptide, an ACT minus peptide, a selectide, or any combination thereof.

14. The milk exosome of claim 1, wherein the milk exosome is a bovine milk exosome.

15. The milk exosome of claim 1, wherein the milk exosome, prior to containing the exogenous cargo, is isolated according to a method comprising:
a. centrifuging a mammalian milk under conditions suitable to separate fats from one or more other components of the mammalian milk;
b. removing the separated fats from the mammalian milk;
c. after step (b) centrifuging the remaining mammalian milk one or more times and skimming any noticeable separated fats after each centrifuging in step (c);
d. filtering the remaining biological fluid after step (c)
e. optionally performing one or more ultracentrifugation steps after (d);
f. chelating divalent cations with about 10 mM to about 100 mM EDTA at about 30-42 degrees Celsius after (d) or optionally (e) and optionally for about 15-120 minutes; and
g. after (f), optionally performing tangential flow filtration to obtain a retentate, wherein the retentate is optionally ultracentrifuged via one or more ultracentrifugation steps or stored at −80 degrees C., and separating out fractions of the retentate, optionally via column separation, after the retentate is optionally ultracentrifuged or stored at −80 degrees C.,
wherein the method comprises step (e) or step (g) but not both.

16. A population of milk exosome comprising one or more milk exosomes as in any one of claims 1-15.

17. The population of milk exosomes of claim 16, wherein the population is enriched for connexin 43 positive milk exosomes.

18. The population of milk exosomes of claim 16, wherein the milk exosome, prior to containing the exogenous cargo, is isolated according to a method comprising
a. centrifuging a mammalian milk under conditions suitable to separate fats from one or more other components of the mammalian milk;
b. removing the separated fats from the mammalian milk;
c. after step (b) centrifuging the remaining mammalian milk one or more times and skimming any noticeable separated fats after each centrifuging in step (c);
d. filtering the remaining biological fluid after step (c)
e. optionally performing one or more ultracentrifugation steps after (d);
f. chelating divalent cations with about 10 mM to about 100 mM EDTA at about 30-42 degrees Celsius after (d) or optionally (e) and optionally for about 15-120 minutes; and
g. after (f), optionally performing tangential flow filtration to obtain a retentate, wherein the retentate is optionally ultracentrifuged via one or more ultracentrifugation steps or stored at −80 degrees C., and separating out fractions of the retentate, optionally via column separation, after the retentate is optionally ultracentrifuged or stored at −80 degrees C.,
wherein the method comprises step (e) or step (g) but not both.

19. The population of milk exosomes of claim 18, further comprising enriching the milk exosomes for connexin 43 expressing milk exosomes.

20. The population of milk exosomes of claim 16, wherein the milk exosomes do not contain an exogenous targeting moiety.

21. The population of milk exosomes of claim 16, wherein the exogenous cargo is a biologic molecule.

22. The population of milk exosomes of claim 21, wherein the biologic molecule is a polypeptide, peptide, or a nucleic acid.

23. The population of milk exosomes of claim 16, wherein the exogenous cargo is an ACT1 peptide, ACT11 peptide, an ACT minus peptide, a selectide, or any combination thereof.

24. A pharmaceutical formulation comprising:
a milk exosome as in claim 1 or a population thereof; and
a pharmaceutically acceptable carrier.

25. The pharmaceutical formulation of claim 24, further comprising an agent capable of stimulating ATP release, secretion, and/or production, ATP, ADP, or any combination thereof.

26. The pharmaceutical formulation of claim 25, wherein the pharmaceutical formulation is in a dosage form that releases the agent capable of stimulating ATP release, secretion, and/or production, ATP, ADP, or any combination thereof prior to the milk exosome or population thereof thereby delivering the agent capable of stimulating ATP release, secretion, and/or production, ATP, ADP, or any combination thereof prior to the milk exosome.

27. The pharmaceutical formulation of claim 24, further comprising one or more immunoglobulins.

28. The pharmaceutical formulation of claim 25, wherein the one or more immunoglobulins are selected from IgG, IgM, IgA, IgD, and/or IgE.

29. The pharmaceutical formulation of claim 27, wherein the pharmaceutical formulation is in a dosage form that releases the immunoglobulin prior to the milk exosome or population thereof thereby delivering the immunoglobulin prior to the milk exosome.

30. A method of treating an injury at an injury site or treating a cancer in a subject in need thereof comprising:
administering to the subject in need thereof, a milk exosome of claim 1 or a population thereof or a pharmaceutical formulation comprising the milk exosome of claim 1 or the population thereof.

31. The method of claim 30, wherein the milk exosome is a bovine milk exosome.

32. The method of claim 30, wherein the milk exosome does not contain an exogenous targeting moiety.

33. The method of claim 30, wherein the milk exosome comprises connexin 43.

34. The method of claim 33, wherein the connexin 43 is a native connexin 43 or wherein the connexin 43 is an engineered connexin 43.

35. The method of claim 30, wherein the exogenous cargo is a biologic molecule.

36. The method of claim 35, wherein the biologic molecule is a polypeptide, peptide, or a nucleic acid.

37. The method of claim 30, wherein the exogenous cargo is an ACT1 peptide, ACT11 peptide, an ACT minus peptide, a selectide, or any combination thereof.

38. The method of claim 30, further comprising administering an amount of one or more immunoglobulins to the subject in need thereof.

39. The method of claim 38, wherein administering the amount of one or more immunoglobulins to subject in need thereof occurs prior to administering the amount of a milk exosome of claim 1 or a population thereof or a pharmaceutical formulation comprising the milk exosome of claim 1 or the population thereof.

40. The method of claim 38, wherein the one or more immunoglobulins are selected from are selected from IgG, IgM, IgA, IgD, and/or IgE.

41. The method of claim 38, wherein the one or more immunoglobulins consists of or comprises IgG.

42. The method of claim 38, wherein the one or more immunoglobulins increase uptake of the milk exosome or population thereof in a cell.

43. The method of claim 42, wherein the cell is a polarized cell.

44. The method of claim 42, wherein the cell is a gastrointestinal cell, a heart cell, a liver cell, brain cell, nerve cell, reproductive cell, cancer cell, or any combination thereof.

45. The method of claim 30, further comprising administering one or more agents that stimulate ATP release, secretion, and/or production, ATP, ADP, or any combination thereof to the subject in need thereof.

46. The method of claim 45, wherein administering the one or more agents that stimulate ATP release, secretion, and/or production, ATP, ADP, or any combination thereof to subject in need thereof occurs prior to administering the amount of a milk exosome of claim 1 or a population thereof or a pharmaceutical formulation comprising the milk exosome of claim 1 or the population thereof or a pharmaceutical formulation comprising the milk exosome of claim 1.

47. The method of claim 45, wherein the one or more agents that stimulate ATP release, secretion, and/or production, ATP, ADP, or any combination thereof increases the cellular uptake of the milk exosome or population thereof in a cell.

48. The method of claim 47, wherein the cell is a polarized cell.

49. The method of claim 47, wherein the cell is a gastrointestinal cell, heart cell, liver cell, brain cell, nerve cell, reproductive cell, cancer cell, or any combination thereof.

50. The method of claim 30, wherein the injury is a mechanical injury site or a non-mechanical injury.

51. The method of claim 50, wherein the non-mechanical injury is a chemical injury, electrical injury, radiation injury, or an ischemic injury.

52. The method of claim 50, wherein the mechanical injury is a wound or burn.

53. The method of claim 30, wherein the milk exosome or population thereof and/or pharmaceutical formulation are irradiated, sterilized, or both.

54. A method of treating a wound or cancer in a subject comprising:
administering a milk exosome and an agent capable of stimulating ATP to the subject in need thereof.

55. The method of claim 54, wherein the milk exosome is as in any one of claims 1-15.

56. The milk exosome, population of milk exosomes, or pharmaceutical formulation of any one of claims 1-29, wherein the milk exosome or population thereof and/or pharmaceutical formulation thereof are irradiated, sterilized, or both.
